(19) United States
(12) Patent Application Publication
(10) Pub. No.: US 2026/0273145 A1
(43) Pub. Date: Sep. 17, 2026

(54) MEDICAL DEVICE THAT INCLUDES A REFRACTORY METAL ALLOY

(71) Applicant: MiRus LLC, Marietta, GA (US)

(72) Inventors: Jay Yadav, Atlanta, GA (US); Noah Roth, Marietta, GA (US)

(21) Appl. No.: 19/674,833

(22) Filed: May 12, 2026

Related U.S. Application Data
(63) Continuation of application No. 17/876,282, filed on Jul. 28, 2022.
Application No. 17/876,282 is a continuation-in-part of application No. 17/586,270, filed on Jan. 27, 2022.
Application No. 17/876,282 is a continuation-in-part of application No. 17/512,174, filed on Oct. 27, 2021, now Pat. No. 11,504,451.
Application No. 17/512,174 is a division of application No. 15/320,830, filed on May 5, 2015, now Pat. No. 11,266,767.
(60) Provisional application No. 62/016,189, filed on Jun. 24, 2014.
(60) Provisional application No. 63/226,270, filed on Jul. 28, 2021.
(60) Provisional application No. 63/226,270, filed on Jul. 28, 2021.
(60) Provisional application No. 63/247,540, filed on Sep. 23, 2021.
(60) Provisional application No. 63/316,077, filed on Mar. 3, 2022.
(60) Provisional application No. 63/389,481, filed on Jul. 15, 2022.

(51) Int. Cl. A61L 31/02; A61L 29/02; C22C 27/00; C22C 27/04; C22F 1/18

(52) U.S. Cl. CPC A61L 31/022; A61L 29/02; C22C 27/00; C22C 27/04; C22F 1/18

(57) ABSTRACT

A medical device that is at least partially formed of a refractory metal alloy, and a method for inserting the medical device in a patient.

The present disclosure is a continuation of U.S. patent application Ser. No. 17/876,282 filed Jul. 28, 2022, which in turn is a continuation in part of U.S. patent application Ser. No. 17/586,270 filed Jan. 27, 2022, which in turn claims priority on U.S. Provisional Application Ser. No. 63/226,270 filed Jul. 28, 2021, which is incorporated herein by reference.

The present disclosure is a continuation of U.S. patent application Ser. No. 17/876,282 filed Jul. 28, 2022, which in turn is a continuation in part of U.S. patent application Ser. No. 17/512,174 filed Oct. 27, 2021, now U.S. Pat. No. 11,504,451 issued Nov. 22, 2022, which in turn is a divisional of U.S. patent application Ser. No. 15/320,830 filed Dec. 21, 2016, now U.S. Pat. No. 11,266,767 issued Mar. 8, 2022, which in turn claims priority to PCT Application Serial No. PCT/US2015/029213 filed May 5, 2015, which in turn claims priority to U.S. Provisional Application Ser. No. 62/016,189 filed Jun. 24, 2014, all of which are incorporated herein by reference.

The present disclosure is continuation of U.S. patent application Ser. No. 17/876,282 filed Jul. 28, 2022, which in turn claims priority on U.S. Provisional Application Ser. No. 63/389,481 filed Jul. 15, 2022, which is incorporated herein by reference.

The present disclosure is continuation of U.S. patent application Ser. No. 17/876,282 filed Jul. 28, 2022, which in turn claims priority on U.S. Provisional Application Ser. No. 63/226,270 filed Jul. 28, 2021, which is incorporated herein by reference.

The present disclosure is continuation of U.S. patent application Ser. No. 17/876,282 filed Jul. 28, 2022, which in turn claims priority on U.S. Provisional Application Ser. No. 63/316,077 filed Mar. 3, 2022, which is incorporated herein by reference.

The present disclosure is continuation of U.S. patent application Ser. No. 17/876,282 filed Jul. 28, 2022, which in turn claims priority on U.S. Provisional Application Ser. No. 63/247,540 filed Sep. 23, 2021, which is incorporated herein by reference.

The disclosure relates generally to medical devices and medical device applications, and more particularly to a medical device that is at least partially formed of a biocompatible metal alloy. In one non-limiting application, the medical device can optionally be in the form into an implant for the treatment of structural heart disease and cardiovascular implants for example a prosthetic heart valve and a transcatheter heart valve that is at least partially formed of the biomedical material such as a biocompatible metal alloy.

### BACKGROUND OF DISCLOSURE

Stainless steel, cobalt-chromium alloys, TiNi alloys, and TiAlV alloys are some of the more common metal alloys used for medical devices. Although these alloys have been successful in forming a variety of medical devices, these alloys have several deficiencies.

Many cardiovascular devices such as stents, expandable heart valves and the like are inserted into a patient via the vascular system of a patient and then expanded at the treatment site. These devices are typically crimped onto catheter prior to insertion into a patient. The minimum diameter to which the cardiovascular device can be crimped onto the catheter will set a limit to the size of the cardiovascular passageway (e.g., blood vessel) to which the cardiovascular device can be inserted. Smaller crimp diameters can result in reduced damage to a blood vessel and/or organ (e.g., heart, etc.) when inserting to and/or placing the cardiovascular device at the treatment site. Smaller crimp diameters can also allow the cardiovascular device to be placed in smaller diameter blood vessels (e.g., blood vessels located in the brain, etc.).

The crimp diameter of the expandable cardiovascular device can be reduced by reducing the thickness and/or size of the frame, struts, strut joints, etc. of the cardiovascular device. However, such reduction in size also affects the strength of the cardiovascular device after being expanded. After the cardiovascular device is expanded, it must retain its expanded shape at the treatment area, otherwise the cardiovascular device could become dislodged from the treatment area, could damage the treatment area, and/or fail to properly function at the treatment area. As such, cardiovascular devices formed of tradition materials such as stainless steel (e.g., 316L: 17-19 wt. % chromium, 13-15 wt. % nickel, 2-4 wt. % molybdenum, 2 wt. % max manganese, 0.75 wt. % max silicon, 0.03 wt. % max carbon, balance iron) and cobalt-chromium alloys (e.g., MP35N: 19-21 wt. % chromium, 34-36 wt. % nickel, 9-11 wt. % molybdenum, 1 wt. % max iron, 1 wt. % max titanium, 0.15 wt. % max manganese, 0.15 wt. % max silicon, 0.025 wt. % max carbon, balance cobalt) are required to maintain a frame and/or strut size/thickness and/or strut joint size/thickness that limits how small of crimping diameter can be obtained by the crimped cardiovascular device. Other types of CoCr alloys that have been used are Phynox and Elgiloy alloy (38-42 wt. % cobalt, 18-22 wt. % chromium, 14-18 wt. % iron, 13-17 wt. % nickel, 6-8 wt. % molybdenum), and L605 alloy (18-22 wt. % chromium, 14-16 wt. % W, 9-11 wt. % nickel, balance cobalt).

Also, tradition materials such as stainless steel (316L) and cobalt-chromium alloys (e.g., MP35N, etc.) have a degree of recoil after being crimped and expanded that can interfere with obtaining a minimum crimping diameter and/or can adversely affect the placement of the expandable cardiovascular device at a treatment area. During a crimping process, a crimping device is typically used to crimp the cardiovascular device onto a catheter. After an initial crimping process, tradition materials such as stainless steel and cobalt-chromium alloys coil to a larger diameter by 9+% of the minimum crimped diameter. As such, the cardiovascular device must be crimped multiple times onto a catheter to attempt to obtain a smaller crimped diameter on the catheter. However, subjecting the cardiovascular device can result is damage to the cardiovascular device (e.g., damage to the frame and/or struts and/or strut joints of the cardiovascular device, damage to leaflets on an expandable heart valve, etc.). Likewise, when the cardiovascular device is expanded at a treatment area, the traditional materials of the cardiovascular device will recoil 9+% of the maximum expanded diameter. As such, the inflatable balloon on the catheter must be pressurized multiple times to repeatedly expand the cardiovascular device at the treatment area to ensure proper expansion of the cardiovascular device. However, subjecting the cardiovascular device to multiple balloon expansions can result in damage to the cardiovascular device (e.g., damage or breakage of a frame and/or strut and/or strut joints, etc.) and/or damage to the treatment area (e.g., rupture of blood vessel, tear and/or puncture of tissue of an organ, etc.).

When a medical device is inserted into a patient, it is typically desirable for the medical device to resist ionization and/or corrosion while in the patient so as to not subject the patient to metal ions and/or oxides from the metals used to form the medical device while in the patient. Excessive ion release from the medical device can potentially be adverse to the patient. Although tradition materials such as stainless steel (316L) and cobalt-chromium alloys (e.g., MP35N, etc.) are very stable when inserted into patients, some degree of metal ion release occurs when the medical device is in the patient.

Medical devices such as Transcatheter aortic valves (TAVs) represent a significant advancement in prosthetic heart valve technology. TAVs bring the benefit of heart valve replacement to patients that would otherwise not be operated on. Transcatheter aortic valve replacement (TAVR) can be used to treat aortic valve stenosis in patients who are classified as high-risk for open heart surgical aortic valve replacement (SAVR). Non-limiting TAVs are disclosed in U.S. Pat. Nos. 5,411,522; 6,730,118; 10,729,543; 10,820,993; 10,856,970; 10,869,761; 10,952,852; 10,980,632; 10,980,633; and US 2020/0405482, all of which are incorporated fully herein by reference. The frame material used to form the TAV is typically CoCr alloy or Nitinol. The vast majority of cardiovascular implants include valves that are made at least impart by using a CoCr alloy or Nitinol materials for construction of the structural frame of the valve.

A TAV is designed to be compressed into a small diameter catheter, remotely placed within a patient's diseased aortic valve so as to take over the function of the native valve. Some TAVs are balloon-expandable, while others are self-expandable. In both cases, the TAVs are deployed within a calcified native valve that is forced permanently open and becomes the surface against which the stent is held in place by friction. TAVs can also be used to replace failing bioprosthetic or transcatheter valves, commonly known as a valve in valve procedure. Major TAVR advantages to the traditional surgical approaches include refraining cardiopulmonary bypass, aortic cross-clamping and sternotomy that significantly reduces patients' morbidity.

However, several complications are associated with current TAV devices such as mispositioning, crimp-induced leaflet damage, paravalvular leak, thrombosis, conduction abnormalities and prosthesis-patient mismatch. These complications are potentially associated with the calcification landscape of the native valve, geometric and mechanical properties of the aortic root, blood biochemistry and coagulability associated with the patient, and concomitant conditions such as hypertension, coronary artery disease, heart failure, etc.

Some limitations of current TAVs that prevent its use in lower risks patients are a) vascular complications from large delivery systems, which necessitates smaller profiles, b) paravalvular leak, c) device mispositioning, and d) device failure. TAVR involves delivery, deployment, and implantation of a crimped, stented valve within a diseased aortic valve or degenerated bioprosthesis, some limitation of these procedures is the diameter to which the stent can be crimped without damaging the leaflet tissues within, vascular complications such as dissection due to the size of the delivery system, and/or recoil associated with the valve frame as defined as the frame being opened to a certain positional diameter and then relaxing or settling to a smaller diameter post implantation which can lead to valve embolization and/or paravalvular leak. Damage to the leaflet tissue can result in increased calcification and early failure of the TAV.

In view of the current state of the art of medical devices, there is a need for an improved medical device that a) produces less recoil as compared to medical devices formed of stainless steel, cobalt-chromium alloys, TiNi alloys, or TiAlV alloys, b) can form smaller crimping diameters as compared to medical devices formed of stainless steel, cobalt-chromium alloys, TiNi alloys, or TiAlV alloys, c) has reduced metal ion release as compared to medical devices formed of stainless steel, cobalt-chromium alloys, TiNi alloys, or TiAlV alloys, and/or d) addresses some of the deficiencies of prior art expandable devices such as, but not limited to, stents, TAVs and the like.

### SUMMARY OF THE DISCLOSURE

The present disclosure is direct to a medical device that is at least partially made of a biomedical material that includes a refractory metal alloy, and more particularly to a prosthetic heart valve that is at least partially formed of a biomedical medical that includes a refractory metal alloy. As defined herein, a refractory metal alloy is a metal alloy that includes at least 20 wt. % of one or more of molybdenum, rhenium, niobium, tantalum or tungsten. Non-limiting refractory metal alloys include MoRe alloy, ReW alloy, MoReCr alloy, MoReTa alloy, MoReTi alloy, WCu alloy, ReCr, molybdenum alloy, rhenium alloy, tungsten alloy, tantalum alloy, niobium alloy, etc.

In accordance with one non-limiting aspect of the present disclosure, the medical device can include, but is not limited to, an orthopedic device, PFO (patent foramen ovale) device, stent, valve (e.g., heart valve, TAVR valve, mitral valve replacement, tricuspid valve replacement, pulmonary valve replacement, etc.), spinal implant, spinal discs, frame and other structures for use with a spinal implant, vascular implant, graft, guide wire, sheath, catheter, needle, stent catheter, electrophysiology catheter, hypotube, staple, cutting device, any type of implant, pacemaker, dental implant, dental crown, dental braces, wire used in medical procedures, bone implant, artificial disk, artificial spinal disk, prosthetic implant or device to repair, replace and/or support a bone (e.g., acromion, atlas, axis, calcaneus, carpus, clavicle, coccyx, epicondyle, epitrochlea, femur, fibula, frontal bone, greater trochanter, humerus, ilium, ischium, mandible, maxilla, metacarpus, metatarsus, occipital bone, olecranon, parietal bone, patella, phalanx, radius, ribs, sacrum, scapula, sternum, talus, tarsus, temporal bone, tibia, ulna, zygomatic bone, etc.) and/or cartilage, bone plate, knee replacement, hip replacement, shoulder replacement, ankle replacement, nail, rod, screw, post, cage, plate, pedicle screw, cap, hinge, joint system, anchor, spacer, shaft, anchor, disk, ball, tension band, locking connector other structural assembly that is used in a body to support a structure, mount a structure, and/or repair a structure in a body such as, but not limited to, a human body, animal body, etc.

In accordance with one non-limiting aspect of the present disclosure, there is provided a medical device in the non-limiting form of a prosthetic heart valve (e.g., TAV valve, mitral valve replacement, tricuspid valve replacement, pulmonary valve replacement). The prosthetic heart valve includes a radially collapsible and expandable frame and a leaflet structure comprising a plurality of leaflets. The prosthetic heart valve can optionally include an annular skirt or cover member disposed on and covering the cells of at least a portion of the frame. The frame can comprise a plurality of interconnected struts and strut joints defining a plurality of open cells in the frame. The frame is partially (e.g., 40-99.99% and all values and ranges therebetween) or fully made of a refractory metal alloy.

In accordance with another and/or alternative non-limiting aspect of the present disclosure, there is provided a prosthetic heart valve that includes a frame or stent, a leaflet structure supported by the frame, and an optional inner skirt secured to the surface of the frame and/or leaflet structure. The prosthetic heart valve can be implanted in the annulus of the native aortic valve; however, the prosthetic heart valve also can be configured to be implanted in other valves of the heart (e.g., tricuspid valve, pulmonary valve, mitral valve). The prosthetic heart valve has a “lower” end and an “upper” end, wherein the lower end of the prosthetic heart valve is the inflow end and the upper end of the prosthetic heart valve is the outflow end.

In accordance with another and/or alternative non-limiting aspect of the present disclosure, there is provided a medical device partially or fully formed of a refractory metal alloy. Non-limiting refractory metal alloys include MoRe alloy, ReW alloy, MoReCr alloy, MoReTa alloy, MoReTi alloy, WCu alloy, ReCr alloy, Mo alloy, Re alloy, W alloy, Ta alloy, Nb alloy, etc. In one non-limiting embodiment, 50-100% (and all values and ranges therebetween) of the medical device is formed of the refractory metal alloy. In another non-limiting embodiment, 50-100% (and all values and ranges therebetween) of the medical device is formed of a MoRe alloy. In another non-limiting embodiment, at least 30 wt. % (e.g., 30-100 wt. % and all values and ranges therebetween) of the refractory metal alloy includes one or more of molybdenum, rhenium, niobium, tantalum or tungsten. In another non-limiting embodiment, the medical device is a prosthetic heart valve and the frame of the prosthetic heart valve is partially or fully formed of a refractory metal alloy. In one non-limiting embodiment, 50-100% (and all values and ranges therebetween) of the frame of the prosthetic heart valve is formed of a refractory metal alloy. In another non-limiting embodiment, 50-100% (and all values and ranges therebetween) of the frame of the prosthetic heart valve is formed of a MoRe alloy.

In accordance with another and/or alternative non-limiting aspect of the present disclosure, the refractory metal alloy that is used to form at least a portion of the medical device is configured to be radially collapsible to a collapsed or crimped state for introduction into the body (e.g., on a delivery catheter, etc.) and radially expandable to an expanded state for implanting the medical device at a desired location in the body (e.g., the aortic valve, tricuspid valve, pulmonary valve, mitral valve, blood vessel, ureter, bile duct, pancreatic duct, esophagus, lung, eyes, sinus, oral stent, etc.). The frame of the medical device can be formed of a plastically-expandable material that permits crimping of the frame to a smaller profile for delivery and expansion of the frame. The expansion of the crimped frame of the medical device can be by an expansion device such as, but not limited to, a balloon of on a balloon catheter.

In accordance with another and/or alternative non-limiting aspect of the present disclosure, there is provided a medical device that includes a frame at least partially formed of a plurality of angularly spaced, vertically extending posts, or struts. The posts or struts can optionally be interconnected via a lower row of circumferentially extending struts and an upper row of circumferentially extending struts via strut joints. The struts can be arrangement in a variety of patterns (e.g., zig-zag pattern, saw-tooth pattern, triangular pattern, polygonal pattern, oval pattern, etc.). One or more of the posts and/or struts can have the same or different thicknesses and/or cross-sectional shape and/or cross-sectional area.

In accordance with another and/or alternative non-limiting aspect of the present disclosure, there is provided a medical device that includes a frame that can be optionally coated with a polymer material (e.g., silicone, PTFE, ePTFE, polyurethane, polyolefins, hydrogels, biological materials (e.g., pericardium or biological polymers such as collagen, gelatin, or hyaluronic acid derivatives), etc.). The coating can be used to partially or fully encapsulate the struts on the frame and/or to fill-in the openings between the struts on the frame.

In accordance with another and/or alternative non-limiting aspect of the present disclosure, there is provided a medical device in the form of a prosthetic heart valve. The prosthetic heart valve can be configured such that it can be crimped so the prosthetic heart valve has a crimped diameter of less than 24 FR (less than 8 mm). Most prior art prosthetic heart values can only be crimped to a diameter of about 24-27 FR (8-9 mm) or larger.

In accordance with another and/or alternative non-limiting aspect of the present disclosure, there is provided a medical device in the form of a prosthetic heart valve that includes an inner skirt that can be formed of a variety of flexible materials (e.g., polymer [e.g., polyethylene terephthalate (PET), polyester, nylon, Kevlar®, silicon, etc.], composite material, metal, fabric material, etc.). In one non-limiting embodiment, the material used to partially or fully form the inner skirt can optionally be substantially non-elastic (i.e., substantially non-stretchable and non-compressible). In another non-limiting embodiment, the material used to partially or fully form the inner skirt can optionally be a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The inner skirt can optionally be formed from a combination of a cloth or fabric material that is coated with a flexible material or with a stretchable and/or compressible material so as to provide additional structural integrity to the inner skirt. The size, configuration, and thickness of the inner skirt is non-limiting (e.g., thickness of 0.1-20 mils and all values and ranges therebetween). The inner skirt can be secured to the inside and/or outside of the frame using various means (e.g., sutures, clamp arrangement, etc.).

In accordance with another and/or alternative non-limiting aspect of the present disclosure, there is provided a medical device in the formed of a prosthetic heart valve that optionally includes an inner skirt that can be used to 1) at least partially seal and/or prevent perivalvular leakage, 2) at least partially secure the leaflet structure to the frame, 3) at least partially protect the leaflets from damage during the crimping and/or expansion process, and/or 4) at least partially protect the leaflets from damage during the operation of the prosthetic heart valve in the heart.

In accordance with another and/or alternative non-limiting aspect of the present disclosure, there is provided a medical device in the form of a prosthetic heart valve that optionally includes an outer or sleeve that is positioned at least partially about the exterior region of the frame. The outer skirt or sleeve generally is positioned completely around a portion of the outside of the frame. Generally, the outer skirt is positioned about the lower portion of the frame, but does not fully cover the upper half of the frame; however, this is not required. The outer skirt can be connected to the frame by a variety of arrangements (e.g., sutures, adhesive, melted connection, clamping arrangement, etc.). At least a portion of the outer skirt can optionally be located on the interior surface of the frame. Generally, the outer skirt is formed of a more flexible and/or compressible material than the inner skirt; however, this is not required. The outer skirt can be formed of a variety of a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The outer skirt can optionally be formed from a combination of a cloth or fabric material that is coated with the stretchable and/or compressible material to provide additional structural integrity to the outer skirt. The size, configuration, and thickness of the outer skirt is non-limiting. The thickness of the outer skirt is generally 0.1-20 mils (and all values and ranges therebetween).

In accordance with another and/or alternative non-limiting aspect of the present disclosure, there is provided a medical device in the form of a prosthetic heart valve that includes a leaflet structure that can be can be attached to the frame and/or skirt. The connection arrangement used to secure the leaflet structures to the frame and/or skirt is non-limiting (e.g., sutures, melted bold, adhesive, clamp arrangement, etc.). The material used to form the leaflet structures include bovine pericardial tissue, biocompatible synthetic materials, or various other suitable natural or synthetic materials.

In accordance with another and/or alternative non-limiting aspect of the present disclosure, there is provided a medical device in the form of a prosthetic heart valve that includes a leaflet structure comprised of two or more leaflets (e.g., 2, 3, 4, 5, 6, etc.). In one non-limiting arrangement, the leaflet structure includes three leaflets arranged to collapse in a tricuspid arrangement. The configuration of the leaflet structures is non-limiting.

In accordance with another and/or alternative non-limiting aspect of the present disclosure, there is provided a medical device in the form of a prosthetic heart valve that includes a leaflet structure wherein the leaflets of the leaflet structure can optionally be secured to one another at their adjacent sides to form commissures of the leaflet structure (the edges where the leaflets come together). The leaflet structure can be secured together by a variety of connection arrangement (e.g., sutures, adhesive, melted bond, clamping arrangement, etc.).

In accordance with another and/or alternative non-limiting aspect of the present disclosure, there is provided a medical device in the form of a prosthetic heart valve that includes a leaflet structure wherein one or more of the leaflets can optionally include reinforcing structures or strips to 1) facilitate in securing the leaflets together, 2) facilitate in securing the leaflets to the skirt and/or frame, and/or 3) inhibit or prevent tearing or other types of damage to the leaflets.

In accordance with another and/or alternative non-limiting aspect of the present disclosure, there is provided a method for crimping a medical device having a frame. The method includes placing the medical device in the crimping aperture of a crimping device such that the frame of the medical device is disposed adjacent to the crimping jaws of the crimping device. Pressure is applied against the frame with the crimping jaws to radially crimp the medical device to a smaller profile.

In accordance with another and/or alternative non-limiting aspect of the present disclosure, the refractory metal alloy that is used to form at least a portion of the medical device has one or more improved properties (e.g., strength, durability, hardness, biostability, bendability, coefficient of friction, radial strength, flexibility, tensile strength, tensile elongation, longitudinal lengthening, stress-strain properties, reduced recoil, radiopacity, heat sensitivity, biocompatibility, improved fatigue life, crack resistance, crack propagation resistance, reduced magnetic susceptibility, etc.), improved conformity when bent, less recoil, less foreshortening, increase yield strength, improved fatigue ductility, improved durability, improved fatigue life, reduced adverse tissue reactions, reduced metal ion release, reduced corrosion, reduced allergic reaction, improved hydrophilicity, reduced toxicity, reduced thickness of metal component, improved bone fusion, and/or lower ion release into tissue. These one or more improved physical properties of the refractory metal alloy can be achieved in the medical device or portion of the medical device (e.g., frame of the medical device, etc.) without having to increase the bulk, volume, and/or weight of the medical device or portion of the medical device (e.g., frame of the medical device, etc.), and in some instances these improved physical properties can be obtained even when the volume, bulk, and/or weight of the medical device or portion of the medical device (e.g., frame of the medical device, etc.) is reduced as compared to medical devices or the frame of the medical device that are at least partially formed from traditional stainless steel, titanium alloy, or cobalt and chromium alloy materials.

In accordance with another and/or alternative non-limiting aspect of the present disclosure, the refractory metal alloy used to at least partially form the medical device or portion of the medical device (e.g., frame of the medical device, etc.) can thus 1) increase the radiopacity of the medical device or portion of the medical device (e.g., frame of the medical device, etc.), 2) increase the radial strength of the medical device or portion of the medical device (e.g., frame of the medical device, etc.), 3) increase the yield strength and/or ultimate tensile strength of the medical device or portion of the medical device (e.g., frame of the medical device, etc.), 4) improve the stress-strain properties of the medical device or portion of the medical device (e.g., frame of the medical device, etc.), 5) improve the crimping and/or expansion properties of the medical device or portion of the medical device (e.g., frame of the medical device, etc.), 6) improve the bendability and/or flexibility of the medical device or portion of the medical device (e.g., frame of the medical device, etc.), 7) improve the strength and/or durability of the medical device or portion of the medical device (e.g., frame of the medical device, etc.), 8) increase the hardness of the medical device or portion of the medical device (e.g., frame of the medical device, etc.), 9) improve the recoil properties of the medical device or portion of the medical device (e.g., frame of the medical device, etc.), 10) improve the biostability and/or biocompatibility properties of the medical device or portion of the medical device (e.g., frame of the medical device, etc.), 11) increase fatigue resistance of the medical device or portion of the medical device (e.g., frame of the medical device, etc.), 12) resist cracking in the medical device or portion of the medical device (e.g., frame of the medical device, etc.) and resist propagation of cracks, 13) enable smaller, thinner, and/or lighter weight medical device or portion of the medical device (e.g., frame of the medical device, etc.) to be made, 14) reduce the outer diameter of a crimped medical device or portion of the medical device (e.g., frame of the medical device, etc.), 15) improve the conformity of the medical device or portion of the medical device (e.g., frame of the medical device, etc.) to the shape of the treatment area when the medical device or portion of the medical device (e.g., frame of the medical device, etc.) is used and/or expanded in the treatment area, 16) reduce the amount of recoil of the medical device or portion of the medical device (e.g., frame of the medical device, etc.) to the shape of the treatment area when the medical device or portion of the medical device (e.g., frame of the medical device, etc.) is expanded in the treatment area, 17) increase yield strength of the medical device or portion of the medical device (e.g., frame of the medical device, etc.), 18) improve fatigue ductility of the medical device or portion of the medical device (e.g., frame of the medical device, etc.), 18) improve durability of the medical device or portion of the medical device (e.g., frame of the medical device, etc.), 19) improve fatigue life of the medical device or portion of the medical device (e.g., frame of the medical device, etc.), 20) reduce adverse tissue reactions after implant of the medical device or portion of the medical device (e.g., frame of the medical device, etc.), 21) reduce metal ion release after implant of the medical device or portion of the medical device (e.g., frame of the medical device, etc.), 22) reduce corrosion of the medical device or portion of the medical device (e.g., frame of the medical device, etc.) after implant of the medical device or portion of the medical device (e.g., frame of the medical device, etc.), 23) reduce allergic reaction after implant of the medical device or portion of the medical device (e.g., frame of the medical device, etc.), 24) improve hydrophilicity of the medical device or portion of the medical device (e.g., frame of the medical device, etc.), 25) reduce thickness of meta component of medical device or portion of the medical device (e.g., frame of the medical device, etc.), 26) improve bone fusion with medical device or portion of the medical device (e.g., frame of the medical device, etc.), and/or 27) lower ion release from medical device or portion of the medical device (e.g., frame of the medical device, etc.) into tissue, 28) reduce magnetic susceptibility of the medical device or portion of the medical device (e.g., frame of the medical device, etc.) when implanted in a patient, 29) reduced foreshortening when the medical device is expanded, and/or 30) reduce toxicity of the medical device or portion of the medical device (e.g., frame of the medical device, etc.) after implant of the prosthetic medical device.

In accordance with another and/or alternative non-limiting aspect of the present disclosure, the medical device or portion of the medical device (e.g., frame of the medical device, etc.) is optionally subjected to one or more manufacturing processes. These manufacturing processes can include, but are not limited to, expansion, laser cutting, etching, crimping, annealing, drawing, pilgering, electroplating, electro-polishing, machining, plasma coating, 3D printing, 3D printed coatings, chemical vapor deposition, chemical polishing, cleaning, pickling, ion beam deposition or implantation, sputter coating, vacuum deposition, etc.

In accordance with another and/or alternative non-limiting aspect of the present disclosure, the refractory metal alloy that is used to at least partially form the medical device or portion of the medical device (e.g., frame of the medical device, etc.) optionally has a generally uniform density throughout the refractory metal alloy, and also results in the desired yield and ultimate tensile strengths of the refractory metal alloy. The density of the refractory metal alloy is generally at least about 5 gm/cc (e.g., 5 gm/cc-21 gm/cc and all values and ranges therebetween; 10-20 gm/cc; etc.), and typically at least about 11-19 gm/cc. This substantially uniform high density of the refractory metal alloy can optionally improve the radiopacity of the refractory metal alloy.

In accordance with another and/or alternative non-limiting aspect of the present disclosure, the refractory metal alloy optionally includes a certain amount of carbon and oxygen; however, this is not required. These two elements have been found to affect the forming properties and brittleness of the refractory metal alloy. The controlled atomic ratio of carbon and oxygen of the refractory metal alloy also can be used to minimize the tendency of the refractory metal alloy to form micro-cracks during the forming of the refractory metal alloy at least partially into a medical device or portion of the medical device (e.g., frame of the medical device, etc.), and/or during the use and/or expansion of the medical device or portion of the medical device (e.g., frame of the medical device, etc.) in a body passageway. The control of the atomic ratio of carbon to oxygen in the refractory metal alloy allows for the redistribution of oxygen in the refractory metal alloy to minimize the tendency of micro-cracking in the refractory metal alloy during the forming of the refractory metal alloy at least partially into a medical device or portion of the medical device (e.g., frame of the medical device, etc.), and/or during the use and/or expansion of the medical device or portion of the medical device (e.g., frame of the medical device, etc.) in a body passageway. The atomic ratio of carbon to oxygen in the refractory metal alloy is believed to facilitate in minimizing the tendency of micro-cracking in the refractory metal alloy and improve the degree of elongation of the refractory metal alloy, both of which can affect one or more physical properties of the refractory metal alloy that are useful or desired in forming and/or using the medical device. The carbon to oxygen atomic ratio can be as low as about 0.2:1 (e.g., 0.2:1 to 50:1 and all values and ranges therebetween). In one non-limiting formulation of the refractory metal alloy, the carbon to oxygen atomic ratio in the refractory metal alloy is generally at least about 0.3:1. Typically, the carbon content of the refractory metal alloy is less than about 0.2 wt. % (e.g., 0 wt. % to 0.1999999 wt. % and all values and ranges therebetween). Carbon contents that are too large can adversely affect the physical properties of the refractory metal alloy. Generally, the oxygen content is to be maintained at very low level. In one non-limiting formulation of the refractory metal alloy, the oxygen content is less than about 0.1 wt. % of the refractory metal alloy (e.g., 0 wt. to 0.0999999 wt. % and all values and ranges therebetween). It is believed that the refractory metal alloy will have a very low tendency to form micro-cracks during the formation of the medical device or portion of the medical device (e.g., frame of the medical device, etc.) and after the medical device has been inserted into a patient by closely controlling the carbon to oxygen ration when the oxygen content exceeds a certain amount in the refractory metal alloy. In one non-limiting arrangement, the carbon to oxygen atomic ratio in the refractory metal alloy is at least about 2.5:1 when the oxygen content is greater than about 100 ppm in the refractory metal alloy.

In accordance with another and/or alternative non-limiting aspect of the present disclosure, the refractory metal alloy optionally includes a controlled amount of nitrogen; however, this is not required. Large amounts of nitrogen in the refractory metal alloy can adversely affect the ductility of the refractory metal alloy. This can in turn adversely affect the elongation properties of the refractory metal alloy. A too high nitrogen content in the refractory metal alloy can begin to cause the ductility of the refractory metal alloy to unacceptably decrease, thus adversely affect one or more physical properties of the refractory metal alloy that are useful or desired in forming and/or using the medical device or frame of the medical device. In one non-limiting formulation, the refractory metal alloy includes less than about 0.001 wt. % nitrogen (e.g., 0 wt. % to −0.0009999 wt. % and all values and ranges therebetween). It is believed that the nitrogen content should be less than the content of carbon or oxygen in the refractory metal alloy. In one non-limiting formulation of the refractory metal alloy, the atomic ratio of carbon to nitrogen is at least about 1.5:1 (e.g., 1.5:1 to 400:1 and all values and ranges therebetween). In another non-limiting formulation of the refractory metal alloy, the atomic ratio of oxygen to nitrogen is at least about 1.2:1 (e.g., 1.2:1 to 150:1 and all value and ranges therebetween).

In another and/or alternative non-limiting aspect of the present disclosure, the medical device or portion of the medical device (e.g., frame of the medical device, etc.) is generally designed to include at least about 5 wt. % of the refractory metal alloy (e.g., 5-100 wt. % and all values and ranges therebetween). In one non-limiting embodiment of the disclosure, the medical device or portion of the medical device (e.g., frame of the medical device, etc.) includes at least about 50 wt. % of the refractory metal alloy. In another non-limiting embodiment of the disclosure, the medical device or portion of the medical device (e.g., frame of the medical device, etc.) includes at least about 95 wt. % of the refractory metal alloy. In one specific configuration, when the medical device includes an expandable frame, the expandable frame is formed of 50-100 wt. % (and all values and ranges therebetween) of the refractory metal alloy, and typically 75-100 wt. % of the refractory metal alloy.

In another and/or alternative non-limiting aspect of the present disclosure, the refractory metal alloy used to form all or part of the medical device 1) is optionally not clad, metal sprayed, plated, and/or formed (e.g., cold worked, hot worked, etc.) onto another metal, or 2) optionally does not have another metal or metal alloy metal sprayed, plated, clad, and/or formed onto the refractory metal alloy.

In accordance with another and/or alternative non-limiting aspect of the present disclosure, the refractory metal alloy that is used to form all or part of the medical device 1) is clad, metal sprayed, plated and/or formed (e.g., cold worked, hot worked, etc.) onto another metal, or 2) has another metal or metal alloy metal sprayed, plated, clad and/or formed onto the refractory metal alloy.

In accordance with another and/or alternative non-limiting aspect of the present disclosure, the medical device or portion of the medical device (e.g., frame of the medical device, etc.) can optionally be at least partially or fully formed from a tube or rod of refractory metal alloy, or be formed into shape that is at least 80% of the final net shape of the medical device or portion of the medical device (e.g., frame of the medical device, etc.).

In accordance with another and/or alternative non-limiting aspect of the present disclosure, the medical device can be at least partially or fully formed from by 3D printing.

In accordance with another and/or alternative non-limiting aspect of the present disclosure, the refractory metal alloy has several physical properties that positively affect the medical device when the medical device is at least partially formed of the refractory metal alloy of the present disclosure. In one non-limiting embodiment of the disclosure, the average Vickers hardness of refractory metal alloy of the present disclosure used to at least partially form the medical device or portion of the medical device (e.g., frame of the medical device, etc.) is optionally at least about 150 Vickers (e.g., 150-300 Vickers and all values and ranges therebetween); and typically 160-240 Vickers; however, this is not required. The refractory metal alloy of the present disclosure generally has an average hardness that is greater than stainless steel (e.g., Grade 304, Grade 316). In another and/or alternative non-limiting embodiment of the disclosure, the average ultimate tensile strength of the refractory metal alloy of the present disclosure is optionally at least about 100 ksi (e.g., 100-350 ksi and all values and ranges therebetween); however, this is not required. In still another and/or alternative non-limiting embodiment of the disclosure, the average yield strength of the refractory metal alloy of the present disclosure is optionally at least about 80 ksi (e.g., 80-300 ksi and all values and ranges therebetween); however, this is not required. In yet another and/or alternative non-limiting embodiment of the disclosure, the average grain size of the refractory metal alloy of the present disclosure used to at least partially form the medical device or portion of the medical device (e.g., frame of the medical device, etc.) is optionally no greater than about 4 ASTM (e.g., 4 ASTM to 20 ASTM using ASTM E112 and all values and ranges therebetween, e.g., 0.35 micron to 90 micron, and all values and ranges therebetween). The small grain size of the refractory metal alloy of the present disclosure enables the medical device or portion of the medical device (e.g., frame of the medical device, etc.) to have the desired elongation and ductility properties that are useful in enabling the medical device or portion of the medical device (e.g., frame of the medical device, etc.) to be formed, crimped, and/or expanded.

In another and/or alternative non-limiting embodiment of the disclosure, the average tensile elongation of the refractory metal alloy of the present disclosure used to at least partially form the medical device or portion of the medical device (e.g., frame of the medical device, etc.) is optionally at least about 25% (e.g., 25%-50% average tensile elongation and all values and ranges therebetween). An average tensile elongation of at least 25% for the refractory metal alloy is useful to facilitate in the medical device or portion of the medical device (e.g., frame of the medical device, etc.) being properly expanded when positioned in the treatment area of a body passageway. A medical device or frame of a medical device that is partially or fully formed of a material that does not have an average tensile elongation of at least about 25% may be more prone to the formation of micro-cracks and/or break during the forming, crimping, and/or expansion of the medical device or portion of the medical device (e.g., frame of the medical device, etc.).

In accordance with another and/or alternative non-limiting aspect of the present disclosure, the unique combination of the metals in the refractory metal alloy of the present disclosure in combination with achieving the desired purity and composition of the refractory metal alloy and the desired grain size of the refractory metal alloy results in 1) a medical device having the desired high ductility at about room temperature, 2) a medical device having the desired amount of tensile elongation, 3) a homogeneous or solid solution of a refractory metal alloy having high radiopacity, 4) a reduction or prevention of micro-crack formation and/or breaking of the refractory metal alloy of the present disclosure tube when the tube is sized and/or cut to form the medical device or portion of the medical device (e.g., frame of the medical device, etc.), 5) a reduction or prevention of micro-crack formation and/or breaking of the medical device or portion of the medical device (e.g., frame of the medical device, etc.) when the medical device or portion of the medical device (e.g., frame of the medical device, etc.) is crimped, 6) a reduction or prevention of micro-crack formation and/or breaking of the medical device or portion of the medical device (e.g., frame of the medical device, etc.) when the medical device is bent and/or expanded in a body passageway, 7) a medical device having the desired ultimate tensile strength and yield strength, 8) a medical device or portion of the medical device (e.g., frame of the medical device, etc.) having very thin wall thicknesses and still having the desired radial forces needed to retain the medical device or portion of the medical device (e.g., frame of the medical device, etc.) on an open state when expanded, 9) a medical device or portion of the medical device (e.g., frame of the medical device, etc.) exhibiting less recoil when the medical device or portion of the medical device (e.g., frame of the medical device, etc.) is crimped onto a delivery system and/or expanded in a body passageway, 10) a medical device exhibiting improved conformity to the shape of the treatment area in the body passageway when the medical device is expanded in a body passageway, 11) a medical device exhibiting improved fatigue ductility, 12) a medical device exhibiting reduced foreshortening when expanded, and/or 13) a medical device that exhibits improved durability.

In accordance with another and/or alternative non-limiting aspect of the present disclosure, at least 30 wt. % (e.g., 30-100 wt. % and all values and ranges therebetween) of the refractory metal alloy includes one or more of molybdenum, niobium, rhenium, tantalum, or tungsten. In another non-limiting embodiment, at least 40 wt. % of the refractory metal alloy includes one or more of molybdenum, niobium, rhenium, tantalum, or tungsten. In another non-limiting embodiment, at least 50 wt. % of the refractory metal alloy includes one or more of molybdenum, niobium, rhenium, tantalum, or tungsten.

In another non-limiting embodiment, at least 50 wt. % (e.g., 50-100 wt. % and all values and ranges therebetween) of the refractory metal alloy includes one or more of molybdenum, niobium, rhenium, tantalum, or tungsten, and 0-40 wt. % (and all values and ranges therebetween) of the refractory alloy includes one or more of calcium, carbon, cerium oxide, chromium, cobalt, copper, gold, hafnium, iridium, iron, lanthanum, lanthanum oxide, lead, magnesium, manganese, nickel, osmium, platinum, rare earth metals, rhodium, ruthenium, silver, technetium, titanium, vanadium, yttrium, yttrium oxide, zinc, zirconium, or zirconium oxide. In another non-limiting embodiment, at least 50 wt. % (e.g., 50-99.9 wt. % and all values and ranges therebetween) of the refractory metal alloy includes one or more of molybdenum, niobium, rhenium, tantalum, or tungsten, and 0.1-40 wt. % (and all values and ranges therebetween) of the refractory alloy includes one or more of calcium, carbon, cerium oxide, chromium, cobalt, copper, gold, hafnium, iridium, iron, lanthanum, lanthanum oxide, lead, magnesium, manganese, nickel, osmium, platinum, rare earth metals, rhodium, ruthenium, silver, technetium, titanium, vanadium, yttrium, yttrium oxide, zinc, zirconium, or zirconium oxide. In another non-limiting embodiment, at least 50 wt. % (e.g., 50-100 wt. % and all values and ranges therebetween) of the refractory metal alloy includes one or more of molybdenum, niobium, rhenium, tantalum, or tungsten, and 0-40 wt. % (and all values and ranges therebetween) of the refractory alloy includes one or more of calcium, carbon, cerium oxide, chromium, cobalt, copper, gold, hafnium, iridium, iron, lanthanum, lanthanum oxide, lead, magnesium, manganese, nickel, osmium, platinum, rare earth metals, rhodium, ruthenium, silver, technetium, titanium, vanadium, yttrium, yttrium oxide, zinc, zirconium, or zirconium oxide, and the refractory alloy includes 0-2 wt. % (and all values and ranges therebetween) of a combination of other metals, carbon, oxygen and nitrogen. In another non-limiting embodiment, at least 50 wt. % (e.g., 50-99.9 wt. % and all values and ranges therebetween) of the refractory metal alloy includes one or more of molybdenum, niobium, rhenium, tantalum, or tungsten, and 0.1-40 wt. % (and all values and ranges therebetween) of the refractory alloy includes one or more of calcium, carbon, cerium oxide, chromium, cobalt, copper, gold, hafnium, iridium, iron, lanthanum, lanthanum oxide, lead, magnesium, manganese, nickel, osmium, platinum, rare earth metals, rhodium, ruthenium, silver, technetium, titanium, vanadium, yttrium, yttrium oxide, zinc, zirconium, or zirconium oxide, and the refractory alloy includes 0-2 wt. % (and all values and ranges therebetween) of a combination of other metals, carbon, oxygen and nitrogen. In another non-limiting embodiment, at least 55 wt. % of the refractory metal alloy includes one or more of molybdenum, niobium, rhenium, tantalum, or tungsten, and 0-40 wt. % of the refractory alloy includes one or more of calcium, carbon, cerium oxide, chromium, cobalt, copper, gold, hafnium, iridium, iron, lanthanum, lanthanum oxide, lead, magnesium, manganese, nickel, osmium, platinum, rare earth metals, rhodium, ruthenium, silver, technetium, titanium, vanadium, yttrium, yttrium oxide, zinc, zirconium, or zirconium oxide, and the refractory alloy includes 0-0.1 wt. % of a combination of other metals, carbon, oxygen and nitrogen. In another non-limiting embodiment, at least 55 wt. % of the refractory metal alloy includes one or more of molybdenum, niobium, rhenium, tantalum, or tungsten, and 0.1-40 wt. % of the refractory alloy includes one or more of calcium, carbon, cerium oxide, chromium, cobalt, copper, gold, hafnium, iridium, iron, lanthanum, lanthanum oxide, lead, magnesium, manganese, nickel, osmium, platinum, rare earth metals, rhodium, ruthenium, silver, technetium, titanium, vanadium, yttrium, yttrium oxide, zinc, zirconium, or zirconium oxide, and the refractory alloy includes 0-0.1 wt. % of a combination of other metals, carbon, oxygen and nitrogen.

In accordance with another and/or alternative non-limiting aspect of the present disclosure, the refractory metal alloy includes at least 30 wt. % (e.g., 30-99 wt. % and all values and ranges therebetween) rhenium and one or more of calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, or zirconium oxide. In another non-limiting embodiment, the refractory metal alloy includes at least 30 wt. % (e.g., 30-99 wt. % and all values and ranges therebetween) rhenium and one or more of calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, or zirconium oxide, and the refractory metal alloy includes 0-2 wt. % (and all values and ranges therebetween) of a combination of other metals, carbon, oxygen, and nitrogen. In another non-limiting embodiment, the refractory metal alloy includes at least 30 wt. % (e.g., 30-99 wt. % and all values and ranges therebetween) rhenium and one or more of calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, or zirconium oxide, and the refractory metal alloy includes 0-0.1 wt. % (and all values and ranges therebetween) of a combination of other metals, carbon, oxygen, and nitrogen. In another non-limiting embodiment, the refractory metal alloy includes at least 35 wt. % (e.g., 35-99 wt. % and all values and ranges therebetween) rhenium and 0.1-65 wt. % (and all values and ranges therebetween) of one or more of calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, or zirconium oxide. In another non-limiting embodiment, the refractory metal alloy includes at least 35 wt. % (e.g., 35-99 wt. % and all values and ranges therebetween) rhenium and 0.1-65 wt. % (and all values and ranges therebetween) of one or more of calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, or zirconium oxide, and the refractory metal alloy includes 0-2 wt. % (and all values and ranges therebetween) of a combination of other metals, carbon, oxygen, and nitrogen. In another non-limiting embodiment, the refractory metal alloy includes at least 35 wt. % (e.g., 35-99.9 wt. % and all values and ranges therebetween) rhenium and 0.1-65 wt. % (and all values and ranges therebetween) of one or more of calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, or zirconium oxide, and the refractory metal alloy includes 0-0.1 wt. % (and all values and ranges therebetween) of a combination of other metals, carbon, oxygen, and nitrogen. In another non-limiting embodiment, the refractory metal alloy includes at least 40 wt. % (e.g., 40-99.9 wt. % and all values and ranges therebetween) rhenium and 0.1-60 wt. % (and all values and ranges therebetween) of one or more of calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, or zirconium oxide. In another non-limiting embodiment, the refractory metal alloy includes at least 40 wt. % (e.g., 40-99.9 wt. % and all values and ranges therebetween) rhenium and 0.1-60 wt. % (and all values and ranges therebetween) of one or more of calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, or zirconium oxide, and the refractory metal alloy includes 0-2 wt. % (and all values and ranges therebetween) of a combination of other metals, carbon, oxygen, and nitrogen. In another non-limiting embodiment, the refractory metal alloy includes at least 40 wt. % (e.g., 40-99.9 wt. % and all values and ranges therebetween) rhenium and 0.1-60 wt. % (and all values and ranges therebetween) of one or more of calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, or zirconium oxide, and the refractory metal alloy includes 0-0.1 wt. % (and all values and ranges therebetween) of a combination of other metals, carbon, oxygen, and nitrogen.

In accordance with another and/or alternative non-limiting aspect of the present disclosure, there is provided a refractory metal alloy wherein at least 20 wt. % (e.g., 20-99 wt. % and all values and ranges therebetween) of the refractory metal alloy includes rhenium. In one non-limiting embodiment, the refractory metal alloy includes at least 20 wt. % (e.g., 20-99.9 wt. % and all values and ranges therebetween) rhenium, and 0.1-80 wt. % (and all values and ranges therebetween) of one or more of calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, zirconium oxide, and/or alloys of one or more of such components. In another non-limiting embodiment, the refractory metal alloy includes at least 20 wt. % (e.g., 30-99.9 wt. % and all values and ranges therebetween) rhenium, and 0.1-80 wt. % (and all values and ranges therebetween) of one or more of copper, chromium, hafnium, iridium, manganese, molybdenum, niobium, osmium, rhodium, ruthenium, tantalum, technetium, titanium, tungsten, vanadium, zirconium, and and/or alloys of one or more of such components. In another non-limiting embodiment, the refractory metal alloy includes at least 30 wt. % (e.g., 30-99.9 wt. % and all values and ranges therebetween) rhenium, and 0.1-70 wt. % (and all values and ranges therebetween) of one or more of calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, zirconium oxide, and/or alloys of one or more of such components. In another non-limiting embodiment, the refractory metal alloy includes at least 30 wt. % (e.g., 30-99.9 wt. % and all values and ranges therebetween) rhenium, and 0.1-70 wt. % (and all values and ranges therebetween) of one or more of copper, chromium, hafnium, iridium, manganese, molybdenum, niobium, osmium, rhodium, ruthenium, tantalum, technetium, titanium, tungsten, vanadium, zirconium, and and/or alloys of one or more of such components. In another non-limiting embodiment, the refractory metal alloy includes at least 35 wt. % (e.g., 35-99.9 wt. % and all values and ranges therebetween) rhenium, and 0.1-65 wt. % (and all values and ranges therebetween) of one or more of calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, zirconium oxide, and/or alloys of one or more of such components. In another non-limiting embodiment, the refractory metal alloy includes at least 35 wt. % (e.g., 35-99.9 wt. % and all values and ranges therebetween) rhenium, and 0.1-65 wt. % (and all values and ranges therebetween) of one or more of copper, chromium, hafnium, iridium, manganese, molybdenum, niobium, osmium, rhodium, ruthenium, tantalum, technetium, titanium, tungsten, vanadium, zirconium, and and/or alloys of one or more of such components. In another non-limiting embodiment, In another non-limiting embodiment, the refractory metal alloy includes 35-60 wt. % (and all values and ranges therebetween) rhenium, and 40-65 wt. % (and all values and ranges therebetween) of one or more of calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, zirconium oxide, and/or alloys of one or more of such components. In another non-limiting embodiment, the refractory metal alloy includes 35-60 wt. % (and all values and ranges therebetween) rhenium, and 40-65 wt. % (and all values and ranges therebetween) of one or more of copper, chromium, hafnium, iridium, manganese, molybdenum, niobium, osmium, rhodium, ruthenium, tantalum, technetium, titanium, tungsten, vanadium, zirconium, and and/or alloys of one or more of such components. In another non-limiting embodiment, the refractory metal alloy includes at least 40 wt. % (e.g., 40-99.9 wt. % and all values and ranges therebetween) rhenium, and 0.1-60 wt. % (and all values and ranges therebetween) of one or more of calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, zirconium oxide, and/or alloys of one or more of such components. In another non-limiting embodiment, the refractory metal alloy includes at least 40 wt. % (e.g., 40-99.9 wt. % and all values and ranges therebetween) rhenium, and 0.1-60 wt. % (and all values and ranges therebetween) of one or more of copper, chromium, hafnium, iridium, manganese, molybdenum, niobium, osmium, rhodium, ruthenium, tantalum, technetium, titanium, tungsten, vanadium, zirconium, and and/or alloys of one or more of such components. In one non-limiting embodiment, the refractory metal alloy includes at least 50 wt. % (e.g., 50-99.9 wt. % and all values and ranges therebetween) rhenium, and 0.1-50 wt. % (and all values and ranges therebetween) of one or more of calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, zirconium oxide, and/or alloys of one or more of such components. In another non-limiting embodiment, the refractory metal alloy includes at least 50 wt. % (e.g., 50-99.9 wt. % and all values and ranges therebetween) rhenium, and 0.1-50 wt. % (and all values and ranges therebetween) of one or more of copper, chromium, hafnium, iridium, manganese, molybdenum, niobium, osmium, rhodium, ruthenium, tantalum, technetium, titanium, tungsten, vanadium, zirconium, and and/or alloys of one or more of such components.

In another non-limiting aspect of the present disclosure, the metals used to form the refractory metal alloy includes rhenium and tungsten and optionally one or more alloying agents such as, but not limited to, calcium, carbon, cerium oxide, chromium, cobalt, copper, gold, hafnium, iron, lanthanum oxide, lead, magnesium, molybdenum, nickel, niobium, osmium, platinum, rare earth metals, rhenium, silver, tantalum, technetium, titanium, vanadium, yttrium, yttrium oxide, zinc, zirconium, zirconium oxide, and/or alloys of one or more of such components (e.g., WRe, WReMo, etc.). Although the refractory metal alloy is described as including one or more metals and/or metal oxides, it can be appreciated that some of the metals and/or metal oxides in the refractory metal alloy can be substituted for one or more materials selected from the group of ceramics, plastics, thermoplastics, thermosets, rubbers, laminates, non-wovens, etc. In one non-limiting formulation, the refractory metal alloy includes 1-40 wt. % rhenium (and all values and ranges therebetween) and 60-99 wt. % tungsten (and all values and ranges therebetween). In one non-limiting embodiment, the total weight percent of the tungsten and rhenium in the tungsten-rhenium alloy is at least about 95 wt. %, typically at least about 99 wt. %, more typically at least about 99.5 wt. %, yet more typically at least about 99.9 wt. %, and still more typically at least about 99.99 wt. %. In another non-limiting formulation, the refractory metal alloy includes 1-47.5 wt. % rhenium (and all values and ranges therebetween) and 20-80 wt. % tungsten (and all values and ranges therebetween) and 1-47.5 wt. % molybdenum (and all values and ranges therebetween). The total weight percent of the tungsten, rhenium, and molybdenum in the tungsten-rhenium-molybdenum alloy is at least about 95 wt. %, typically at least about 99 wt. %, more typically at least about 99.5 wt. %, yet more typically at least about 99.9 wt. %, and still more typically at least about 99.99 wt. %. In one non-limiting specific tungsten-rhenium-molybdenum alloy, the weight percent of the tungsten is greater than a weight percent of rhenium and also greater than the weight percent of molybdenum. In another non-limiting specific tungsten-rhenium-molybdenum alloy, the weight percent of the tungsten is greater than 50 wt. % of the tungsten-rhenium-molybdenum alloy. In another non-limiting specific tungsten-rhenium-molybdenum alloy, the weight percent of the tungsten is greater than a weight percent of rhenium, but less than a weigh percent of molybdenum. In another non-limiting specific tungsten-rhenium-molybdenum alloy, the weight percent of the tungsten is greater than a weight percent of molybdenum, but less than a weigh percent of rhenium. In another non-limiting specific tungsten-rhenium-molybdenum alloy, the weight percent of the tungsten is less than a weight percent of rhenium and also less than the weight percent of molybdenum.

In accordance with another and/or alternative non-limiting aspect of the present disclosure, there is provided a refractory metal alloy wherein at least 30 wt. % (e.g., 30-99 wt. % and all values and ranges therebetween) of the refractory metal alloy includes rhenium. In another non-limiting embodiment, at least 35 wt. % of the refractory metal alloy includes rhenium. In another non-limiting embodiment, at least 35 wt. % (e.g., 35-99.9 wt. % and all values and ranges therebetween) of the refractory metal alloy includes rhenium, and 0.1-65 wt. % (and all values and ranges therebetween) of the refractory metal alloy includes one or more of molybdenum, niobium, tantalum, tantalum, titanium, vanadium, chromium, manganese, zirconium, technetium, ruthenium, rhodium, hafnium, osmium, copper, and/or iridium. In another non-limiting embodiment, 35-60 wt. % (and all values and ranges therebetween) of the refractory metal alloy includes rhenium, and 40-65 wt. % (and all values and ranges therebetween) of the refractory metal alloy includes one or more of molybdenum, niobium, tantalum, tungsten, titanium, vanadium, chromium, manganese, zirconium, technetium, ruthenium, rhodium, hafnium, osmium, copper, and/or iridium. In another non-limiting embodiment, 35-60 wt. % (e.g., and all values and ranges therebetween) of the refractory metal alloy includes rhenium, and 40-65 wt. % (and all values and ranges therebetween) of the refractory metal alloy includes two or more of molybdenum, niobium, tantalum, tantalum, titanium, vanadium, chromium, manganese, zirconium, technetium, ruthenium, rhodium, hafnium, osmium, copper, and/or iridium. In another non-limiting embodiment, 35-60 wt. % (e.g., and all values and ranges therebetween) of the refractory metal alloy includes rhenium, and 40-65 wt. % (and all values and ranges therebetween) of the refractory metal alloy includes three or more of molybdenum, niobium, tantalum, tantalum, titanium, vanadium, chromium, manganese, zirconium, technetium, ruthenium, rhodium, hafnium, osmium, copper, and/or iridium.

In another non-limiting aspect of the present disclosure, the metals used to form the refractory metal alloy include at least 35 wt. % rhenium (e.g., 35-99.9 wt. % and all values and ranges therebetween) and one or more alloying agents such as, but are not limited to, molybdenum, niobium, tantalum, tantalum, titanium, vanadium, chromium, manganese, zirconium, technetium, ruthenium, rhodium, hafnium, osmium, copper, and/or iridium, and/or alloys of one or more of such components. In one non-limiting formulation, the refractory metal alloy includes 40-99.9 wt. % rhenium and one or more molybdenum, niobium, tantalum, tantalum, titanium, vanadium, chromium, manganese, zirconium, technetium, ruthenium, rhodium, hafnium, osmium, copper, and/or iridium. In one non-limiting formulation, the refractory metal alloy includes rhenium and one or more molybdenum, niobium, tantalum, tantalum, titanium, vanadium, chromium, manganese, zirconium, technetium, ruthenium, rhodium, hafnium, osmium, copper, and/or iridium.

In another non-limiting aspect of the present disclosure, the metals used to form the refractory metal alloy include rhenium, molybdenum, and one or more alloying metals selected from the group consisting of bismuth, chromium, copper, hafnium, iridium, manganese, niobium, osmium, rhodium, ruthenium, tantalum, technetium, titanium, tungsten, vanadium, yttrium, and zirconium. In one non-limiting embodiment, a combined weight percentage of rhenium and alloy metals in the refractory metal alloy is greater than or equal to the weight percent of molybdenum in the refractory metal alloy. In another non-limiting embodiment, a combined weight percentage of rhenium and alloy metals in the refractory metal alloy is greater than the weight percent of molybdenum in the refractory metal alloy. In another non-limiting embodiment, a weight percent of molybdenum in the refractory metal alloy is at least 10 wt. % and less than 60 wt. % (and all values and ranges therebetween). In another non-limiting embodiment, a weight percent of rhenium in the refractory metal alloy is 35-60 wt. % (and all values and ranges therebetween). In another non-limiting embodiment, a combined weight percent of the alloying metals is 5-45 wt. % (and all values and ranges therebetween) of the refractory metal alloy. In another non-limiting embodiment, a weight percent of the rhenium in the refractory metal alloy is greater than a combined weight percent of the alloying metals. In another non-limiting embodiment, a combined weight percent of the rhenium, molybdenum, and the one or more alloying metals in the refractory metal alloy is at least 99.9 wt. %. In another non-limiting embodiment, alloy metal includes chromium. In another non-limiting embodiment, the alloying metal includes chromium and one or more metals selected from the group consisting of bismuth, zirconium, iridium, niobium, tantalum, titanium, and yttrium. In another non-limiting embodiment, the alloying metal includes chromium and one or more metals selected from the group consisting of bismuth, zirconium, iridium, niobium, tantalum, titanium, and yttrium; and wherein an atomic ratio of chromium to an atomic ratio of each or all of the metals selected from the group consisting of bismuth, chromium, iridium, niobium, tantalum, titanium, and yttrium is 0.4:1 to 2.5:1 (and all values and ranges therebetween). In another non-limiting embodiment, the alloying metal includes chromium and one or more metals selected from the group consisting of zirconium, niobium, and tantalum. In another non-limiting embodiment, the alloying metal includes a first metal selected from the group consisting of bismuth, chromium, iridium, niobium, tantalum, titanium, yttrium and zirconium, and a second metal selected from the group consisting of bismuth, chromium, iridium, niobium, tantalum, titanium, yttrium and zirconium; and wherein the first and second metals are different; and wherein an atomic ratio of the first metal to the second metal is 0.4:1 to 2.5:1 (and all values and ranges therebetween). In another non-limiting embodiment, the alloying metal a first metal selected from the group consisting of chromium, niobium, tantalum, and zirconium, and a second metal selected from the group consisting of chromium, niobium, tantalum, and zirconium; and wherein the first and second metals are different; and wherein an atomic ratio of the first metal to the second metal is 0.4:1 to 2.5:1 (and all values and ranges therebetween).

In accordance with another and/or alternative non-limiting aspect of the present disclosure, the weight percent of rhenium plus the weigh percent of the combined weight percentage of bismuth, niobium, tantalum, tungsten, titanium, vanadium, chromium, manganese, yttrium, zirconium, technetium, ruthenium, rhodium, hafnium, osmium, copper, and iridium is greater than the weight percent of molybdenum in the refractory metal alloy. In one specific non-limiting formulation, the weight percent of rhenium plus the weigh percent of the combined weight percentage of bismuth, chromium, iridium, niobium, tantalum, titanium, yttrium, and zirconium is greater than the weight percent of molybdenum in the refractory metal alloy. In another specific non-limiting formulation, the weight percent of rhenium plus the weigh percent of the combined weight percentage of chromium, niobium, tantalum, and zirconium is greater than the weight percent of molybdenum in the refractory metal alloy. In another non-limiting specific non-limiting formulation, the weight percent of molybdenum in the refractory metal alloy is at least 10 wt. % and less than 50 wt. % (and all values and ranges therebetween). In another non-limiting specific non-limiting formulation, the weight percent of rhenium in the refractory metal alloy is 41-58.5 wt. % (and all values and ranges therebetween), the weight percent of molybdenum in the refractory metal alloy is at least 15-45 wt. % (and all values and ranges therebetween), and the combined weight percent of bismuth, niobium, tantalum, tungsten, titanium, vanadium, chromium, manganese, yttrium, zirconium, technetium, ruthenium, rhodium, hafnium, osmium, copper, and iridium in the refractory metal alloy is 11-41 wt. % (and all values and ranges therebetween). In another non-limiting specific non-limiting formulation, the weight percent of rhenium in the refractory metal alloy is 41-58.5 wt. % (and all values and ranges therebetween), the weight percent of molybdenum in the refractory metal alloy is at least 15-45 wt. % (and all values and ranges therebetween), and the combined weight percent of bismuth, chromium, iridium, niobium, tantalum, titanium, yttrium, and zirconium in the refractory metal alloy is 11-41 wt. % (and all values and ranges therebetween). In another non-limiting specific non-limiting formulation, the weight percent of rhenium in the refractory metal alloy is 41-58.5 wt. % (and all values and ranges therebetween), the weight percent of molybdenum in the refractory metal alloy is at least 15-45 wt. % (and all values and ranges therebetween), and the combined weight percent of chromium, niobium, tantalum, and zirconium in the refractory metal alloy is 11-41 wt. % (and all values and ranges therebetween). In another non-limiting embodiment of the invention, the weight percent of rhenium in the refractory metal alloy is greater than the combined weight percent of bismuth, chromium, iridium, niobium, tantalum, titanium, yttrium, and zirconium in the refractory metal alloy. In another non-limiting specific non-limiting formulation, the weight percent of rhenium in the refractory metal alloy is greater than the combined weight percent of chromium, niobium, tantalum, and zirconium in the refractory metal alloy.

In accordance with another and/or alternative non-limiting aspect of the present disclosure, the atomic weight percent of rhenium to the atomic weight percent of the combination of bismuth, niobium, tantalum, tungsten, titanium, vanadium, chromium, manganese, yttrium, zirconium, technetium, ruthenium, rhodium, hafnium, osmium, copper, and iridium in the refractory metal alloy is 0.7:1 to 1.5:1 (and all values and ranges therebetween), typically 0.8:1 to 1.4:1, more typically 0.8:1 to 1.25:1, and still more typically about 0.9:1 to 1.1:1 (e.g., 1:1). In one specific non-limiting formulation, the atomic weight percent of rhenium to the atomic weight percent of the combination of bismuth, chromium, iridium, niobium, tantalum, titanium, yttrium, and zirconium is 0.7:1 to 5.1:1 (and all values and ranges therebetween), typically 0.8:1 to 1.5:1, more typically 0.8:1 to 1.25:1, and still more typically about 0.9:1 to 1.1:1 (e.g., 1:1). In one specific non-limiting formulation, the atomic weight percent of rhenium to the atomic weight percent of the combination of chromium, niobium, tantalum, and zirconium is 0.7:1 to 5.1:1 (and all values and ranges therebetween), typically 0.8:1 to 1.5:1, more typically 0.8:1 to 1.25:1, and still more typically about 0.9:1 to 1.1:1 (e.g., 1:1).

In accordance with another and/or alternative non-limiting aspect of the present disclosure, when the refractory metal alloy includes two of bismuth, niobium, tantalum, tungsten, titanium, vanadium, chromium, manganese, yttrium, zirconium, technetium, ruthenium, rhodium, hafnium, osmium, copper, and iridium, the atomic ratio of the two metals is 0.4:1 to 2.5:1 (and all values and ranges therebetween), and typically 0.5:1 to 2:1. In one specific non-limiting formulation, when the refractory metal alloy includes two of bismuth, chromium, iridium, niobium, tantalum, titanium, yttrium, and zirconium, the atomic ratio of the two metals is 0.4:1 to 2.5:1 (and all values and ranges therebetween), and typically 0.5:1 to 2:1. In another specific non-limiting formulation, when the refractory metal alloy includes two of chromium, niobium, tantalum, and zirconium, the atomic ratio of the two metals is 0.4:1 to 2.5:1 (and all values and ranges therebetween), and typically 0.5:1 to 2:1.

In accordance with another and/or alternative non-limiting aspect of the present disclosure, at least 35 wt. % (e.g., 35-75 wt. % and all values and ranges therebetween) of the refractory metal alloy includes rhenium, and the refractory metal alloy also includes chromium. In one non-limiting embodiment, at least 25 wt. % (e.g., 25-49.9 wt. % and all values and ranges therebetween) of the refractory metal alloy includes chromium. In another non-limiting embodiment, at least 30 wt. % of the refractory metal alloy includes chromium. In another non-limiting embodiment, at least 33 wt. % of the refractory metal alloy includes chromium. In another non-limiting embodiment, at least 50 wt. % (e.g., 50-74.9 wt. % and all values and ranges therebetween) of the refractory metal alloy includes rhenium, at least 25 wt. % (e.g., 25-49.9 wt. % and all values and ranges therebetween) of the refractory metal alloy includes chromium, and 0.1-25 wt. % (and all values and ranges therebetween) of the refractory metal alloy includes one or more of molybdenum, bismuth, niobium, tantalum, titanium, vanadium, tungsten, manganese, zirconium, technetium, ruthenium, rhodium, hafnium, osmium, copper, yttrium, zirconium, and/or iridium. In another non-limiting embodiment, at least 55 wt. % (e.g., 55-69.9 wt. % and all values and ranges therebetween) of the refractory metal alloy includes rhenium, at least 30 wt. % (e.g., 30-44.9 wt. % and all values and ranges therebetween) of the refractory metal alloy includes chromium, and 0.1-15 wt. % (and all values and ranges therebetween) of the refractory metal alloy includes one or more of molybdenum, bismuth, niobium, tantalum, titanium, vanadium, tungsten, manganese, zirconium, technetium, ruthenium, rhodium, hafnium, osmium, copper, yttrium, zirconium, and/or iridium. In another non-limiting embodiment, at least 60 wt. % (e.g., 60-69.9 wt. % and all values and ranges therebetween) of the refractory metal alloy includes rhenium, at least 30 wt. % (e.g., 30-39.9 wt. % and all values and ranges therebetween) of the refractory metal alloy includes chromium, and 0.1-10 wt. % (and all values and ranges therebetween) of the refractory metal alloy includes one or more of molybdenum, bismuth, niobium, tantalum, titanium, vanadium, tungsten, manganese, zirconium, technetium, ruthenium, rhodium, hafnium, osmium, copper, yttrium, zirconium, and/or iridium. In another non-limiting embodiment, at least 62 wt. % (e.g., 62-67.9 wt. % and all values and ranges therebetween) of the refractory metal alloy includes rhenium, at least 32 wt. % (e.g., 32-32.9 wt. % and all values and ranges therebetween) of the refractory metal alloy includes chromium, and 0.1-6 wt. % (and all values and ranges therebetween) of the refractory metal alloy includes one or more of molybdenum, bismuth, niobium, tantalum, titanium, vanadium, tungsten, manganese, zirconium, technetium, ruthenium, rhodium, hafnium, osmium, copper, yttrium, zirconium, and/or iridium.

In accordance with another and/or alternative non-limiting aspect of the present disclosure, the refractory metal alloy includes less than about 5 wt. % (e.g., 0-4.999999 wt. % and all values and ranges therebetween) other metals and/or impurities. A high purity level of the refractory metal alloy results in the formation of a more homogeneous alloy, which in turn results in a more uniform density throughout the refractory metal alloy, and also results in the desired yield and ultimate tensile strengths of the refractory metal alloy. In one non-limiting embodiment, the refractory metal alloy includes less than about 0.5 wt. % other metals and/or impurities. In another non-limiting embodiment, the refractory metal alloy includes less than about 0.2 wt. % other metals and/or impurities. In another non-limiting embodiment, the refractory metal alloy includes less than about 0.1 wt. % other metals and/or impurities. In another non-limiting embodiment, the refractory metal alloy includes less than about 0.05 wt. % other metals and/or impurities. In another non-limiting embodiment, the refractory metal alloy includes less than about 0.01 wt. % other metals and/or impurities.

Several non-limiting examples of the refractory metal alloy in accordance with the present disclosure are set forth below:

| | Wt. % | | | |
|---|---|---|---|---|
| Metal | Ex. 1 | Ex. 2 | Ex. 3 | Ex. 4 |
| Mo | 40-80% | 40-80% | 40-80% | 40-80% |
| C | 0.01-0.3% | 0-0.3% | 0-0.3% | 0-0.3% |
| Co | ≤0.002% | ≤0.002% | ≤0.002% | ≤0.002% |
| Cs₂O | 0-0.2% | 0-0.2% | 0.01-0.2% | 0-0.2% |
| Fe | ≤0.02% | ≤0.02% | ≤0.02% | ≤0.02% |
| H | ≤0.002% | ≤0.002% | ≤0.002% | ≤0.002% |
| Hf | 0.1-2.5% | 0-2.5% | 0-2.5% | 0-2.5% |
| O | ≤0.06% | ≤0.06% | ≤0.06% | ≤0.06% |
| Os | ≤1% | ≤1% | ≤1% | ≤1% |
| La₂O₃ | 0-2% | 0.1-2% | 0-2% | 0-2% |
| N | ≤20 ppm | ≤20 ppm | ≤20 ppm | ≤20 ppm |
| Nb | ≤0.01% | ≤0.01% | ≤0.01% | ≤0.01% |
| Pt | ≤1% | ≤1% | ≤1% | ≤1% |
| Re | 20-49% | 20-49% | 20-49% | 20-49% |
| S | ≤0.008% | ≤0.008% | ≤0.008% | ≤0.008% |
| Sn | ≤0.002% | ≤0.002% | ≤0.002% | ≤0.002% |
| Ta | 0-50% | 0-50% | 0-50% | 0-50% |
| Tc | ≤1% | ≤1% | ≤1% | ≤1% |
| Ti | ≤1% | ≤1% | ≤1% | ≤1% |
| V | ≤1% | ≤1% | ≤1% | ≤1% |
| W | 0-50% | 0-50% | 0-50% | 0.5-50% |
| Y₂O₃ | 0-1% | 0-1% | 0.1-1% | 0-1% |
| Zr | ≤1% | ≤1% | ≤1% | ≤1% |
| ZrO₂ | 0-3% | 0-3% | 0-3% | 0-3% |
| CNT | 0-10% | 0-10% | 0-10% | 0-10% |

| | Wt. % | | |
|---|---|---|---|
| Metal | Ex. 5 | Ex. 6 | Ex. 7 |
| Mo | 40-80% | 40-80% | 40-80% |
| C | 0-0.3% | 0-0.3% | 0-0.3% |
| Co | ≤0.002% | ≤0.002% | ≤0.002% |
| Cs₂O | 0-0.2% | 0-0.2% | 0-0.2% |
| H | ≤0.002% | ≤0.002% | ≤0.002% |
| Hf | 0-2.5% | 0-2.5% | 0-2.5% |
| O | ≤0.06% | ≤0.06% | ≤0.06% |
| Os | ≤1% | ≤1% | ≤1% |
| La₂O₃ | 0-2% | 0-2% | 0-2% |
| N | ≤20 ppm | ≤20 ppm | ≤20 ppm |
| Nb | ≤0.01% | ≤0.01% | ≤0.01% |
| Pt | ≤1% | ≤1% | ≤1% |
| Re | 20-49% | 20-49% | 20-49% |
| S | ≤0.008% | ≤0.008% | ≤0.008% |
| Sn | ≤0.002% | ≤0.002% | ≤0.002% |
| Ta | 0-50% | 0.5-50% | 0-50% |
| Tc | ≤1% | ≤1% | ≤1% |
| Ti | ≤1% | ≤1% | ≤1% |
| V | ≤1% | ≤1% | ≤1% |
| W | 0-50% | 0-50% | 0-50% |
| Y₂O₃ | 0-1% | 0-1% | 0-1% |
| ZrO₂ | 0.1-3% | 0-3% | 0-3% |
| CNT | 0-10% | 0-10% | 0-10% |
| | Wt. % | | |
| Metal | Ex. 8 | Ex. 9 | Ex. 10 |
| Mo | 45-78% | 45-75% | 45-70% |
| C | 0-0.3% | 0-0.3% | 0-0.3% |
| Co | ≤0.002% | ≤0.002% | ≤0.002% |
| Cs₂O | 0-0.2% | 0-0.2% | 0-0.2% |
| H | ≤0.002% | ≤0.002% | ≤0.002% |
| Hf | 0-2.5% | 0-2.5% | 0-2.5% |
| O | ≤0.06% | ≤0.06% | ≤0.06% |
| Os | ≤1% | ≤1% | ≤1% |
| La₂O₃ | 0-2% | 0-2% | 0-2% |
| N | ≤20 ppm | ≤20 ppm | ≤20 ppm |
| Nb | ≤0.01% | ≤0.01% | ≤0.01% |
| Pt | ≤1% | ≤1% | ≤1% |
| Re | 22-49% | 25-49% | 30-49% |
| S | ≤0.008% | ≤0.008% | ≤0.008% |
| Sn | ≤0.002% | ≤0.002% | ≤0.002% |
| Ta | 0-50% | 0.5-50% | 0-50% |
| Tc | ≤1% | ≤1% | ≤1% |
| Ti | ≤1% | ≤1% | ≤1% |
| V | ≤1% | ≤1% | ≤1% |
| W | 0-50% | 0-50% | 0-50% |
| Y₂O₃ | 0-1% | 0-1% | 0-1% |
| ZrO₂ | 0.1-3% | 0-3% | 0-3% |
| CNT | 0-10% | 0-10% | 0-10% |

| | Wt. % | | | |
|---|---|---|---|---|
| Metal | Ex. 11 | Ex. 12 | Ex. 13 | Ex. 14 |
| Mo | 35-80% | 35-80% | 35-70% | 35-65% |
| C | 0.05-0.15% | 0-0.15% | 0-0.15% | 0-0.15% |
| Cs₂O | 0-0.2% | 0-0.2% | 0.04-0.1% | 0-0.2% |
| Hf | 0.8-1.4% | 0-2% | 0-2.5% | 0-2.5% |
| La₂O₃ | 0-2% | 0.3-0.7% | 0-2% | 0-2% |
| Re | 20-49% | 20-49% | 30-49% | 35-49% |
| Ta | 0-2% | 0-2% | 0-50% | 0-50% |
| W | 0-2% | 0-2% | 0-50% | 20-50% |
| Y₂O₃ | 0-1% | 0-1% | 0.3-0.5% | 0-1% |
| ZrO₂ | 0-3% | 0-3% | 0-3% | 0-3% |

| | Wt. % | | |
|---|---|---|---|
| Metal | Ex. 15 | Ex. 16 | Ex. 17 |
| Mo | 40-60% | 35-60% | 30-60% |
| C | 0-0.15% | 0-0.15% | 0-0.15% |
| Cs₂O | 0-0.2% | 0-0.2% | 0-0.2% |
| Hf | 0-2.5% | 0-2.5% | 0-2.5% |
| La₂O₃ | 0-2% | 0-2% | 0-2% |
| Re | 40-60% | 40-65% | 40-70% |
| Ta | 0-3% | 10-50% | 0-40% |
| W | 0-3% | 0-50% | 0-40% |
| Y₂O₃ | 0-1% | 0-1% | 0-1% |
| ZrO₂ | 1.2-1.8% | 0-3% | 0-3% |

| | Wt. % | | |
|---|---|---|---|
| Metal | Ex. 18 | Ex. 19 | Ex. 20 |
| W | 20-80% | 60-80% | 20-78% |
| Re | 20-47.5% | 20-40% | 22-47.5% |
| Mo | 0-47.5% | <0.5% | 1-47.5% |
| Cu | <0.5% | <0.5% | <0.5% |
| C | ≤0.15% | ≤0.15% | ≤0.15% |
| Co | ≤0.002% | ≤0.002% | ≤0.002% |
| Cs₂O | ≤0.2% | ≤0.2% | ≤0.2% |
| Fe | ≤0.02% | ≤0.02% | ≤0.02% |
| H | ≤0.002% | ≤0.002% | ≤0.002% |
| Hf | <0.5% | <0.5% | <0.5% |
| La₂O₃ | <0.5% | <0.5% | <0.5% |
| O | ≤0.06% | ≤0.06% | ≤0.06% |
| Os | <0.5% | <0.5% | <0.5% |
| N | ≤20 ppm | ≤20 ppm | ≤20 ppm |
| Nb | ≤0.01% | ≤0.01% | ≤0.01% |
| Pt | <0.5% | <0.5% | <0.5% |
| S | ≤0.008% | ≤0.008% | ≤0.008% |
| Sn | ≤0.002% | ≤0.002% | ≤0.002% |
| Ta | <0.5% | <0.5% | <0.5% |
| Tc | <0.5% | <0.5% | <0.5% |
| Ti | <0.5% | <0.5% | <0.5% |
| V | <0.5% | <0.5% | <0.5% |
| Y₂O₃ | <0.5% | <0.5% | <0.5% |
| Zr | <0.5% | <0.5% | <0.5% |
| ZrO₂ | <0.5% | <0.5% | <0.5% |
| CNT | 0-10% | 0-10% | <0.5%. |

| | Wt. % | | |
|---|---|---|---|
| Metal | Ex. 21 | Ex. 22 | Ex. 23 |
| W | 20-80% | 60-80% | 20-75% |
| Re | 20-47.5% | 20-40% | 25-47.5% |
| Mo | 0-47.5% | <0.5% | 1-47.5% |
| | Wt. % | | |
| Metal | Ex. 24 | Ex. 25 | Ex. 26 |
| W | 50.1-80% | 65-80% | 50.1-79% |
| Re | 20-40% | 20-35% | 20-40% |
| Mo | 0-40% | <0.5% | 1-30% |

| | Wt. % | | |
|---|---|---|---|
| Metal | Ex. 27 | Ex. 28 | Ex. 29 |
| W | 20-49% | 20-49% | 20-49% |
| Re | 20-60% | 30-60% | 40-60% |
| Mo | 0-40% | 0-40% | 0-39% |

| | Wt. % | | |
|---|---|---|---|
| Metal | Ex. 30 | Ex. 31 | Ex. 32 |
| Re | 20-98% | 60-95% | 80-90% |
| Mo | 0-80% | 0-40% | 0-20% |
| W | 0-80% | 0-40% | 0-20% |

| | Wt. % | | |
|---|---|---|---|
| Metal | Ex. 33 | Ex. 34 | Ex. 35 |
| W | 20-49% | 20-49% | 20-49% |
| Re | 20-40% | 20-40% | 22-39% |
| Mo | 20-60% | 30-60% | 40-60% |
| | Wt. % | | |
| Metal | Ex. 36 | Ex. 37 | Ex. 38 |
| W | 20-40% | 20-35% | 20-30% |
| Re | 20-40% | 20-40% | 31-40% |
| Mo | 0-40% | 10-40% | 31-40% |

| | Wt. % | | | |
|---|---|---|---|---|
| | Ex. 39 | Ex. 40 | Ex. 41 | Ex. 42 |
| Re | 30-60% | 35-60% | 35-60% | 35-60% |
| Mo | 0-55% | 10-55% | 10-55% | 10-55% |
| Bi | 1-42 | 0-32 | 0-32 | 0-32 |
| Cr | 0-32 | 1-42 | 0-32 | 0-32 |
| Ir | 0-32 | 0-32 | 1-42 | 0-32 |
| Nb | 0-32 | 0-32 | 0-32 | 1-42 |
| Ta | 0-32 | 0-32 | 0-32 | 0-32 |
| Ti | 0-32 | 0-32 | 0-32 | 0-32 |
| Y | 0-32 | 0-32 | 0-32 | 0-32 |
| Zr | 0-32 | 0-32 | 0-32 | 0-32 |
| C | <0.06 | <0.06 | <0.06 | <0.06 |
| N | <0.06 | <0.06 | <0.06 | <0.06 |
| O | <0.06 | <0.06 | <0.06 | <0.06 |
| | Wt. % | | | |
| | Ex. 43 | Ex. 44 | Ex. 45 | Ex. 46 |
| Re | 30-60% | 35-60% | 35-60% | 35-60% |
| Mo | 15-55% | 15-55% | 15-55% | 15-55% |
| Bi | 0-32 | 0-32 | 0-32 | 0-32 |
| Cr | 0-32 | 0-32 | 0-32 | 0-32 |
| Ir | 0-32 | 0-32 | 0-32 | 0-32 |
| Nb | 0-32 | 0-32 | 0-32 | 0-32 |
| Ta | 1-42 | 0-32 | 0-32 | 0-32 |
| Ti | 0-32 | 1-42 | 0-32 | 0-32 |
| Y | 0-32 | 0-32 | 1-42 | 0-32 |
| Zr | 0-32 | 0-32 | 0-32 | 1-42 |
| C | <0.06 | <0.06 | <0.06 | <0.06 |
| N | <0.06 | <0.06 | <0.06 | <0.06 |
| O | <0.06 | <0.06 | <0.06 | <0.06 |
| | Wt. % | | | |
| | Ex. 47 | Ex. 48 | Ex. 49 | Ex. 50 |
| Re | 41-59% | 41-59% | 41-59% | 41-59% |
| Mo | 18-45% | 18-45% | 18-45% | 18-45% |
| Bi | 1-42 | 0-32 | 0-32 | 0-32 |
| Cr | 0-32 | 1-42 | 0-32 | 0-32 |
| Ir | 0-32 | 0-32 | 1-42 | 0-32 |
| Nb | 0-32 | 0-32 | 0-32 | 1-42 |
| Ta | 0-32 | 0-32 | 0-32 | 0-32 |
| Ti | 0-32 | 0-32 | 0-32 | 0-32 |
| Y | 0-32 | 0-32 | 0-32 | 0-32 |
| Zr | 0-32 | 0-32 | 0-32 | 0-32 |
| C | <0.06 | <0.06 | <0.06 | <0.06 |
| N | <0.06 | <0.06 | <0.06 | <0.06 |
| O | <0.06 | <0.06 | <0.06 | <0.06 |
| | Wt. % | | | |
| | Ex. 51 | Ex. 52 | Ex. 53 | Ex. 54 |
| Re | 41-59% | 41-59% | 41-59% | 41-59% |
| Mo | 18-45% | 18-45% | 18-45% | 18-45% |
| Bi | 0-32 | 0-32 | 0-32 | 0-32 |
| Cr | 0-32 | 0-32 | 0-32 | 0-32 |
| Ir | 0-32 | 0-32 | 0-32 | 0-32 |
| Nb | 0-32 | 0-32 | 0-32 | 0-32 |
| Ta | 1-42 | 0-32 | 0-32 | 0-32 |
| Ti | 0-32 | 1-42 | 0-32 | 0-32 |
| Y | 0-32 | 0-32 | 1-42 | 0-32 |
| Zr | 0-32 | 0-32 | 0-32 | 1-42 |
| C | <0.06 | <0.06 | <0.06 | <0.06 |
| N | <0.06 | <0.06 | <0.06 | <0.06 |
| O | <0.06 | <0.06 | <0.06 | <0.06 |
| | Wt. % | | | |
| | Ex. 55 | Ex. 56 | Ex. 57 | Ex. 58 |
| Re | 41-59% | 41-59% | 41-59% | 41-59% |
| Mo | 18-45% | 18-45% | 18-45% | 18-45% |
| Bi | 0-15 | 0-15 | 1-36 | 0-15 |
| Cr | 1-20 | 1-20 | 1-20 | 1-20 |
| Ir | 0-15 | 0-15 | 0-15 | 0-15 |
| Nb | 1-36 | 0-15 | 0-15 | 0-15 |
| Ta | 0-15 | 1-36 | 0-15 | 0-15 |
| Ti | 0-15 | 0-15 | 0-15 | 0-15 |
| Y | 0-15 | 0-15 | 0-15 | 0-15 |
| Zr | 0-15 | 0-15 | 0-15 | 1-36 |
| C | <0.06 | <0.06 | <0.06 | <0.06 |
| N | <0.06 | <0.06 | <0.06 | <0.06 |
| O | <0.06 | <0.06 | <0.06 | <0.06 |
| | Wt. % | | | |
| | Ex. 59 | Ex. 60 | Ex. 61 | Ex. 62 |
| Re | 41-59% | 41-59% | 41-59% | 41-59% |
| Mo | 18-45% | 18-45% | 18-45% | 18-45% |
| Bi | 1-36 | 0-15 | 0-15 | 0-15 |
| Cr | 1-20 | 1-20 | 1-20 | 1-20 |
| Ir | 0-15 | 1-36 | 0-15 | 0-15 |
| Nb | 0-15 | 0-15 | 0-15 | 0-15 |
| Ta | 0-15 | 0-15 | 0-15 | 0-15 |
| Ti | 0-15 | 0-15 | 1-36 | 0-15 |
| Y | 0-15 | 0-15 | 0-15 | 1-36 |
| Zr | 0-15 | 0-15 | 0-15 | 0-15 |
| C | <0.06 | <0.06 | <0.06 | <0.06 |
| N | <0.06 | <0.06 | <0.06 | <0.06 |
| O | <0.06 | <0.06 | <0.06 | <0.06 |
| | Wt. % | | | |
| | Ex. 63 | Ex. 64 | Ex. 65 | Ex. 66 |
| Re | 41-59% | 41-59% | 41-59% | 41-59% |
| Mo | 18-45% | 18-45% | 18-45% | 18-45% |
| Bi | 1-34 | 0-15 | 0-15 | 0-15 |
| Cr | 0-15 | 0-15 | 0-15 | 0-15 |
| Ir | 0-15 | 0-15 | 0-15 | 1-34 |
| Nb | 3-27 | 3-27 | 3-27 | 3-27 |
| Ta | 0-42 | 1-34 | 0-15 | 0-15 |
| Ti | 0-15 | 0-15 | 0-15 | 0-15 |
| Y | 0-15 | 0-15 | 0-15 | 0-15 |
| Zr | 0-15 | 0-15 | 3-27 | 0-15 |
| C | <0.06 | <0.06 | <0.06 | <0.06 |
| N | <0.06 | <0.06 | <0.06 | <0.06 |
| O | <0.06 | <0.06 | <0.06 | <0.06 |
| | Wt. % | | | |
| | Ex. 67 | Ex. 68 | Ex. 69 | Ex. 70 |
| Re | 41-59% | 41-59% | 41-59% | 41-59% |
| Mo | 18-45% | 18-45% | 18-45% | 18-45% |
| Bi | 0-15 | 0-15 | 0-15 | 0-15 |
| Cr | 0-15 | 0-15 | 0-15 | 0-15 |
| Ir | 0-15 | 1-34 | 0-15 | 0-15 |
| Nb | 0-15 | 0-15 | 0-15 | 0-15 |
| Ta | 1-34 | 0-15 | 3-27 | 0-15 |
| Ti | 0-15 | 0-15 | 0-15 | 0-15 |
| Y | 0-15 | 0-15 | 0-15 | 3-27 |
| Zr | 3-27 | 3-27 | 3-27 | 3-27 |
| C | <0.06 | <0.06 | <0.06 | <0.06 |
| N | <0.06 | <0.06 | <0.06 | <0.06 |
| O | <0.06 | <0.06 | <0.06 | <0.06 |
| | Wt. % | | | |
| | Ex. 71 | Ex. 72 | Ex. 73 | Ex. 74 |
| Re | 41-59% | 41-59% | 41-59% | 41-59% |
| Mo | 18-45% | 18-45% | 18-45% | 18-45% |
| Bi | 0-15 | 0-15 | 0-15 | 0-15 |
| Cr | 0-15 | 0-15 | 0-15 | 1-10 |
| Ir | 1-34 | 0-25 | 3-27 | 0-15 |
| Nb | 0-15 | 3-27 | 0-15 | 0-15 |
| Ta | 0-15 | 0-15 | 1-34 | 0-15 |
| Ti | 0-15 | 0-15 | 0-15 | 0-15 |
| Y | 3-27 | 3-27 | 0-15 | 0-15 |
| Zr | 0-15 | 0-15 | 3-27 | 1-12 |
| C | <0.06 | <0.06 | <0.06 | <0.06 |
| N | <0.06 | <0.06 | <0.06 | <0.06 |
| O | <0.06 | <0.06 | <0.06 | <0.06 |

| | Wt. % | | | |
|---|---|---|---|---|
| Element | Ex. 75 | Ex. 76 | Ex. 77 | Ex. 78 |
| Re | 50-75% | 55-75% | 60-75% | 65-75% |
| Cr | 25-50% | 25-45% | 25-40% | 25-35% |
| Mo | 0-25% | 0-25% | 0-25% | 0-25% |
| Bi | 0-25% | 0-25% | 0-25% | 0-25% |
| Cr | 0-25% | 0-25% | 0-25% | 0-25% |
| Ir | 0-25% | 0-25% | 0-25% | 0-25% |
| Nb | 0-25% | 0-25% | 0-25% | 0-25% |
| Ta | 0-25% | 0-25% | 0-25% | 0-25% |
| V | 0-25% | 0-25% | 0-25% | 0-25% |
| W | 0-25% | 0-25% | 0-25% | 0-25% |
| Mn | 0-25% | 0-25% | 0-25% | 0-25% |
| Tc | 0-25% | 0-25% | 0-25% | 0-25% |
| Ru | 0-25% | 0-25% | 0-25% | 0-25% |
| Rh | 0-25% | 0-25% | 0-25% | 0-25% |
| Hf | 0-25% | 0-25% | 0-25% | 0-25% |
| Os | 0-25% | 0-25% | 0-25% | 0-25% |
| Cu | 0-25% | 0-25% | 0-25% | 0-25% |
| Ir | 0-25% | 0-25% | 0-25% | 0-25% |
| Ti | 0-25% | 0-25% | 0-25% | 0-25% |
| Y | 0-25% | 0-25% | 0-25% | 0-25% |
| Zr | 0-25% | 0-25% | 0-25% | 0-25% |
| C | <0.06 | <0.06 | <0.06 | <0.06 |
| N | <0.06 | <0.06 | <0.06 | <0.06 |
| O | <0.06 | <0.06 | <0.06 | <0.06 |
| | Wt. % | | | |
| Element | Ex. 79 | Ex. 80 | Ex. 81 | Ex. 82 |
| Re | 50-72% | 55-72% | 60-72% | 65-72% |
| Cr | 28-50% | 28-45% | 28-40% | 28-35% |
| Mo | 0-25% | 0-25% | 0-25% | 0-25% |
| Bi | 0-10% | 0-10% | 0-10% | 0-10% |
| Cr | 0-10% | 0-10% | 0-10% | 0-10% |
| Ir | 0-10% | 0-10% | 0-10% | 0-10% |
| Nb | 0-10% | 0-10% | 0-10% | 0-10% |
| Ta | 0-10% | 0-10% | 0-10% | 0-10% |
| V | 0-10% | 0-10% | 0-10% | 0-10% |
| W | 0-10% | 0-10% | 0-10% | 0-10% |
| Mn | 0-10% | 0-10% | 0-10% | 0-10% |
| Tc | 0-10% | 0-10% | 0-10% | 0-10% |
| Ru | 0-10% | 0-10% | 0-10% | 0-10% |
| Rh | 0-10% | 0-10% | 0-10% | 0-10% |
| Hf | 0-10% | 0-10% | 0-10% | 0-10% |
| Os | 0-10% | 0-10% | 0-10% | 0-10% |
| Cu | 0-10% | 0-10% | 0-10% | 0-10% |
| Ir | 0-10% | 0-10% | 0-10% | 0-10% |
| Ti | 0-10% | 0-10% | 0-10% | 0-10% |
| Y | 0-10% | 0-10% | 0-10% | 0-10% |
| Zr | 0-10% | 0-10% | 0-10% | 0-10% |
| C | <0.06 | <0.06 | <0.06 | <0.06 |
| N | <0.06 | <0.06 | <0.06 | <0.06 |
| O | <0.06 | <0.06 | <0.06 | <0.06 |
| | Wt. % | | | |
| Element | Ex. 83 | Ex. 84 | Ex. 85 | Ex. 86 |
| Re | 50-70% | 55-70% | 60-70% | 65-70% |
| Cr | 30-50% | 30-45% | 30-40% | 30-35% |
| Mo | 0-10% | 0-10% | 0-10% | 0-10% |
| Bi | 0-10% | 0-10% | 0-10% | 0-10% |
| Cr | 0-10% | 0-10% | 0-10% | 0-10% |
| Ir | 0-10% | 0-10% | 0-10% | 0-10% |
| Nb | 0-10% | 0-10% | 0-10% | 0-10% |
| Ta | 0-10% | 0-10% | 0-10% | 0-10% |
| V | 0-10% | 0-10% | 0-10% | 0-10% |
| W | 0-10% | 0-10% | 0-10% | 0-10% |
| Mn | 0-10% | 0-10% | 0-10% | 0-10% |
| Tc | 0-10% | 0-10% | 0-10% | 0-10% |
| Ru | 0-10% | 0-10% | 0-10% | 0-10% |
| Rh | 0-10% | 0-10% | 0-10% | 0-10% |
| Hf | 0-10% | 0-10% | 0-10% | 0-10% |
| Os | 0-10% | 0-10% | 0-10% | 0-10% |
| Cu | 0-10% | 0-10% | 0-10% | 0-10% |
| Ir | 0-10% | 0-10% | 0-10% | 0-10% |
| Ti | 0-10% | 0-10% | 0-10% | 0-10% |
| Y | 0-10% | 0-10% | 0-10% | 0-10% |
| Zr | 0-10% | 0-10% | 0-10% | 0-10% |
| C | <0.06 | <0.06 | <0.06 | <0.06 |
| N | <0.06 | <0.06 | <0.06 | <0.06 |
| O | <0.06 | <0.06 | <0.06 | <0.06 |

| | Wt. % | | | |
|---|---|---|---|---|
| Element | Ex. 87 | Ex. 88 | Ex. 89 | Ex. 90 |
| Re | 50-67.5% | 55-67.5% | 60-67.5% | 65-67.5% |
| Cr | 32.5-50% | 32.5-45% | 32.5-40% | 32.5-35% |
| Mo | 0-10% | 0-10% | 0-10% | 0-10% |
| Bi | 0-10% | 0-10% | 0-10% | 0-10% |
| Cr | 0-10% | 0-10% | 0-10% | 0-10% |
| Ir | 0-10% | 0-10% | 0-10% | 0-10% |
| Nb | 0-10% | 0-10% | 0-10% | 0-10% |
| Ta | 0-10% | 0-10% | 0-10% | 0-10% |
| V | 0-10% | 0-10% | 0-10% | 0-10% |
| W | 0-10% | 0-10% | 0-10% | 0-10% |
| Mn | 0-10% | 0-10% | 0-10% | 0-10% |
| Tc | 0-10% | 0-10% | 0-10% | 0-10% |
| Ru | 0-10% | 0-10% | 0-10% | 0-10% |
| Rh | 0-10% | 0-10% | 0-10% | 0-10% |
| Hf | 0-10% | 0-10% | 0-10% | 0-10% |
| Os | 0-10% | 0-10% | 0-10% | 0-10% |
| Cu | 0-10% | 0-10% | 0-10% | 0-10% |
| Ir | 0-10% | 0-10% | 0-10% | 0-10% |
| Ti | 0-10% | 0-10% | 0-10% | 0-10% |
| Y | 0-10% | 0-10% | 0-10% | 0-10% |
| Zr | 0-10% | 0-10% | 0-10% | 0-10% |
| C | <0.06 | <0.06 | <0.06 | <0.06 |
| N | <0.06 | <0.06 | <0.06 | <0.06 |
| O | <0.06 | <0.06 | <0.06 | <0.06 |

| | Wt. % | | | |
|---|---|---|---|---|
| Element | Ex. 91 | Ex. 92 | Ex. 93 | Ex. 94 |
| Re | 50-67.5% | 55-67.5% | 60-67.5% | 65-67.5% |
| Cr | 32.5-50% | 32.5-45% | 32.5-40% | 32.5-35% |
| Mo | 0-5% | 0-5% | 0-5% | 0-5% |
| Bi | 0-5% | 0-5% | 0-5% | 0-5% |
| Cr | 0-5% | 0-5% | 0-5% | 0-5% |
| Ir | 0-5% | 0-5% | 0-5% | 0-5% |
| Nb | 0-5% | 0-5% | 0-5% | 0-5% |
| Ta | 0-5% | 0-5% | 0-5% | 0-5% |
| V | 0-5% | 0-5% | 0-5% | 0-5% |
| W | 0-5% | 0-5% | 0-5% | 0-5% |
| Mn | 0-5% | 0-5% | 0-5% | 0-5% |
| Tc | 0-5% | 0-5% | 0-5% | 0-5% |
| Ru | 0-5% | 0-5% | 0-5% | 0-5% |
| Rh | 0-5% | 0-5% | 0-5% | 0-5% |
| Hf | 0-5% | 0-5% | 0-5% | 0-5% |
| Os | 0-5% | 0-5% | 0-5% | 0-5% |
| Cu | 0-5% | 0-5% | 0-5% | 0-5% |
| Ir | 0-5% | 0-5% | 0-5% | 0-5% |
| Ti | 0-5% | 0-5% | 0-5% | 0-5% |
| Y | 0-5% | 0-5% | 0-5% | 0-5% |
| Zr | 0-5% | 0-5% | 0-5% | 0-5% |
| C | <0.06 | <0.06 | <0.06 | <0.06 |
| N | <0.06 | <0.06 | <0.06 | <0.06 |
| O | <0.06 | <0.06 | <0.06 | <0.06 |

| | Wt. % | | | |
|---|---|---|---|---|
| Metal | Ex. 95 | Ex. 96 | Ex. 97 | Ex. 98 |
| Mo | 40-99.98% | 40-99.9% | 40-99.8% | 40-99.5% |
| C | 0.01-0.3% | 0-0.3% | 0-0.3% | 0-0.3% |
| Co | ≤0.002% | ≤0.002% | ≤0.002% | ≤0.002% |
| Cs₂O | 0-0.2% | 0-0.2% | 0.01-0.2% | 0-0.2% |
| Fe | ≤0.02% | ≤0.02% | ≤0.02% | ≤0.02% |
| H | ≤0.002% | ≤0.002% | ≤0.002% | ≤0.002% |
| Hf | 0.1-2.5% | 0-2.5% | 0-2.5% | 0-2.5% |
| O | ≤0.06% | ≤0.06% | ≤0.06% | ≤0.06% |
| Os | ≤1% | ≤1% | ≤1% | ≤1% |
| La₂O₃ | 0-2% | 0.1-2% | 0-2% | 0-2% |
| N | ≤20 ppm | ≤20 ppm | ≤20 ppm | ≤20 ppm |
| Nb | ≤0.01% | ≤0.01% | ≤0.01% | ≤0.01% |
| Pt | ≤1% | ≤1% | ≤1% | ≤1% |
| Re | 0-40% | 0-40% | 0-40% | 0-40% |
| S | ≤0.008% | ≤0.008% | ≤0.008% | ≤0.008% |
| Sn | ≤0.002% | ≤0.002% | ≤0.002% | ≤0.002% |
| Ta | 0-50% | 0-50% | 0-50% | 0-50% |
| Tc | ≤1% | ≤1% | ≤1% | ≤1% |
| Ti | ≤1% | ≤1% | ≤1% | ≤1% |
| V | ≤1% | ≤1% | ≤1% | ≤1% |
| W | 0-50% | 0-50% | 0-50% | 0.5-50% |
| Y₂O₃ | 0-1% | 0-1% | 0.1-1% | 0-1% |
| Zr | ≤1% | ≤1% | ≤1% | ≤1% |
| ZrO₂ | 0-3% | 0-3% | 0-3% | 0-3% |
| CNT | 0-10% | 0-10% | 0-10% | 0-10% |

| | Wt. % | | |
|---|---|---|---|
| Metal | Ex. 99 | Ex. 100 | Ex. 101 |
| Mo | 40-99.9% | 40-99.5% | 40-99.5% |
| C | 0-0.3% | 0-0.3% | 0-0.3% |
| Co | ≤0.002% | ≤0.002% | ≤0.002% |
| Cs₂O | 0-0.2% | 0-0.2% | 0-0.2% |
| H | ≤0.002% | ≤0.002% | ≤0.002% |
| Hf | 0-2.5% | 0-2.5% | 0-2.5% |
| O | ≤0.06% | ≤0.06% | ≤0.06% |
| Os | ≤1% | ≤1% | ≤1% |
| La₂O₃ | 0-2% | 0-2% | 0-2% |
| N | ≤20 ppm | ≤20 ppm | ≤20 ppm |
| Nb | ≤0.01% | ≤0.01% | ≤0.01% |
| Pt | ≤1% | ≤1% | ≤1% |
| Re | 0-40% | 0-40% | 0.5-40% |
| S | ≤0.008% | ≤0.008% | ≤0.008% |
| Sn | ≤0.002% | ≤0.002% | ≤0.002% |
| Ta | 0-50% | 0.5-50% | 0-50% |
| Tc | ≤1% | ≤1% | ≤1% |
| Ti | ≤1% | ≤1% | ≤1% |
| V | ≤1% | ≤1% | ≤1% |
| W | 0-50% | 0-50% | 0-50% |
| Y₂O₃ | 0-1% | 0-1% | 0-1% |
| ZrO₂ | 0.1-3% | 0-3% | 0-3% |
| CNT | 0-10% | 0-10% | 0-10% |
| | Wt. % | | |
| Metal | Ex. 102 | Ex. 103 | Ex. 104 |
| Mo | 60-99% | 60-95% | 60-90% |
| C | 0-0.3% | 0-0.3% | 0-0.3% |
| Co | ≤0.002% | ≤0.002% | ≤0.002% |
| Cs₂O | 0-0.2% | 0-0.2% | 0-0.2% |
| H | ≤0.002% | ≤0.002% | ≤0.002% |
| Hf | 0-2.5% | 0-2.5% | 0-2.5% |
| O | ≤0.06% | ≤0.06% | ≤0.06% |
| Os | ≤1% | ≤1% | ≤1% |
| La₂O₃ | 0-2% | 0-2% | 0-2% |
| N | ≤20 ppm | ≤20 ppm | ≤20 ppm |
| Nb | ≤0.01% | ≤0.01% | ≤0.01% |
| Pt | ≤1% | ≤1% | ≤1% |
| Re | 1-40% | 5-40% | 10-40% |
| S | ≤0.008% | ≤0.008% | ≤0.008% |
| Sn | ≤0.002% | ≤0.002% | ≤0.002% |
| Ta | 0-50% | 0.5-50% | 0-50% |
| Tc | ≤1% | ≤1% | ≤1% |
| Ti | ≤1% | ≤1% | ≤1% |
| V | ≤1% | ≤1% | ≤1% |
| W | 0-50% | 0-50% | 0-50% |
| Y₂O₃ | 0-1% | 0-1% | 0-1% |
| ZrO₂ | 0.1-3% | 0-3% | 0-3% |
| CNT | 0-10% | 0-10% | 0-10% |

| | Wt. % | | | |
|---|---|---|---|---|
| Metal | Ex. 105 | Ex. 106 | Ex. 107 | Ex. 108 |
| Mo | 98-99.15% | 98-99.7% | 50-99.66% | 40-80% |
| C | 0.05-0.15% | 0-0.15% | 0-0.15% | 0-0.15% |
| Cs₂O | 0-0.2% | 0-0.2% | 0.04-0.1% | 0-0.2% |
| Hf | 0.8-1.4% | 0-2% | 0-2.5% | 0-2.5% |
| La₂O₃ | 0-2% | 0.3-0.7% | 0-2% | 0-2% |
| Re | 0-2% | 0-2% | 0-40% | 0-40% |
| Ta | 0-2% | 0-2% | 0-50% | 0-50% |
| W | 0-2% | 0-2% | 0-50% | 20-50% |
| Y₂O₃ | 0-1% | 0-1% | 0.3-0.5% | 0-1% |
| ZrO₂ | 0-3% | 0-3% | 0-3% | 0-3% |

| | Wt. % | | |
|---|---|---|---|
| Metal | Ex. 109 | Ex. 110 | Ex. 111 |
| Mo | 97-98.8% | 50-90% | 60-99.5% |
| C | 0-0.15% | 0-0.15% | 0-0.15% |
| Cs₂O | 0-0.2% | 0-0.2% | 0-0.2% |
| Hf | 0-2.5% | 0-2.5% | 0-2.5% |
| La₂O₃ | 0-2% | 0-2% | 0-2% |
| Re | 0-3% | 0-40% | 0.5-40% |
| Ta | 0-3% | 10-50% | 0-40% |
| W | 0-3% | 0-50% | 0-40% |
| Y₂O₃ | 0-1% | 0-1% | 0-1% |
| ZrO₂ | 1.2-1.8% | 0-3% | 0-3% |
| | Wt. % | | |
| Metal | Ex. 112 | Ex. 113 | Ex. 114 |
| W | 20-99% | 60-99% | 20-80% |
| Re | 1-47.5% | 1-40% | 1-47.5% |
| Mo | 0-47.5% | <0.5% | 1-47.5% |
| Cu | <0.5% | <0.5% | <0.5% |
| C | ≤0.15% | ≤0.15% | ≤0.15% |
| Co | ≤0.002% | ≤0.002% | ≤0.002% |
| Cs₂O | ≤0.2% | ≤0.2% | ≤0.2% |
| Fe | ≤0.02% | ≤0.02% | ≤0.02% |
| H | ≤0.002% | ≤0.002% | ≤0.002% |
| Hf | <0.5% | <0.5% | <0.5% |
| La₂O₃ | <0.5% | <0.5% | <0.5% |
| O | ≤0.06% | ≤0.06% | ≤0.06% |
| Os | <0.5% | <0.5% | <0.5% |
| N | ≤20 ppm | ≤20 ppm | ≤20 ppm |
| Nb | ≤0.01% | ≤0.01% | ≤0.01% |
| Pt | <0.5% | <0.5% | <0.5% |
| S | ≤0.008% | ≤0.008% | ≤0.008% |
| Sn | ≤0.002% | ≤0.002% | ≤0.002% |
| Ta | <0.5% | <0.5% | <0.5% |
| Tc | <0.5% | <0.5% | <0.5% |
| Ti | <0.5% | <0.5% | <0.5% |
| V | <0.5% | <0.5% | <0.5% |
| Y₂O₃ | <0.5% | <0.5% | <0.5% |
| Zr | <0.5% | <0.5% | <0.5% |
| ZrO₂ | <0.5% | <0.5% | <0.5% |
| CNT | 0-10% | 0-10% | <0.5%. |

| | Wt. % | | | |
|---|---|---|---|---|
| Metal | Ex. 115 | Ex. 116 | Ex. 117 | Ex. 118 |
| W | 1-99.9% | 1-99.9% | 1-99.9% | 10-99% |
| Cu | 0.1-99% | 0.1-99% | 0.1-99% | 1-90% |
| C | 0.01-0.3% | 0-0.3% | 0-0.3% | 0-0.3% |
| Co | ≤0.002% | ≤0.002% | ≤0.002% | ≤0.002% |
| Cs₂O | 0-0.2% | 0-0.2% | 0.01-0.2% | 0-0.2% |
| Fe | ≤0.02% | ≤0.02% | ≤0.02% | ≤0.02% |
| H | ≤0.002% | ≤0.002% | ≤0.002% | ≤0.002% |
| Hf | 0.1-2.5% | 0-2.5% | 0-2.5% | 0-2.5% |
| O | ≤0.06% | ≤0.06% | ≤0.06% | ≤0.06% |
| Os | ≤1% | ≤1% | ≤1% | ≤1% |
| La₂O₃ | 0-2% | 0.1-2% | 0-2% | 0-2% |
| Mo | 0-5% | 0.1-3% | 0-2% | 0-3% |
| N | ≤20 ppm | ≤20 ppm | ≤20 ppm | ≤20 ppm |
| Nb | ≤0.01% | ≤0.01% | ≤0.01% | ≤0.01% |
| Pt | ≤1% | ≤1% | ≤1% | ≤1% |
| Re | 0-40% | 0-40% | 0-40% | 0-40% |
| S | ≤0.008% | ≤0.008% | ≤0.008% | ≤0.008% |
| Sn | ≤0.002% | ≤0.002% | ≤0.002% | ≤0.002% |
| Ta | 0-50% | 0-50% | 0-50% | 0-50% |
| Tc | ≤1% | ≤1% | ≤1% | ≤1% |
| Ti | ≤1% | ≤1% | ≤1% | ≤1% |
| V | ≤1% | ≤1% | ≤1% | ≤1% |
| Y₂O₃ | 0-1% | 0-1% | 0.1-1% | 0-1% |
| Zr | ≤1% | ≤1% | ≤1% | ≤1% |
| ZrO₂ | 0-3% | 0-3% | 0-3% | 0-3% |
| CNT | 0-10% | 0-10% | 0-10% | 0-10% |

| | Wt. % | | |
|---|---|---|---|
| Metal | Ex. 119 | Ex. 120 | Ex. 121 |
| W | 20-98% | 25-98% | 30-95% |
| Cu | 2-80% | 2-75% | 5-70% |
| C | 0-0.3% | 0-0.3% | 0-0.3% |
| Co | ≤0.002% | ≤0.002% | ≤0.002% |
| Cs₂O | 0-0.2% | 0-0.2% | 0-0.2% |
| H | ≤0.002% | ≤0.002% | ≤0.002% |
| Hf | 0-2.5% | 0-2.5% | 0-2.5% |
| O | ≤0.06% | ≤0.06% | ≤0.06% |
| Os | ≤1% | ≤1% | ≤1% |
| La₂O₃ | 0-2% | 0-2% | 0-2% |
| Mo | 0-3% | 0-2% | 0-1% |
| N | ≤20 ppm | ≤20 ppm | ≤20 ppm |
| Nb | ≤0.01% | ≤0.01% | ≤0.01% |
| Pt | ≤1% | ≤1% | ≤1% |
| Re | 0-40% | 0-40% | 0.5-40% |
| S | ≤0.008% | ≤0.008% | ≤0.008% |
| Sn | ≤0.002% | ≤0.002% | ≤0.002% |
| Ta | 0-50% | 0.5-50% | 0-50% |
| Tc | ≤1% | ≤1% | ≤1% |
| Ti | ≤1% | ≤1% | ≤1% |
| V | ≤1% | ≤1% | ≤1% |
| Y₂O₃ | 0-1% | 0-1% | 0-1% |
| ZrO₂ | 0.1-3% | 0-3% | 0-3% |
| CNT | 0-10% | 0-10% | 0-10% |

| | Wt. % | | | |
|---|---|---|---|---|
| Metal | Ex. 122 | Ex. 123 | Ex. 124 | Ex. 125 |
| W | 25-95% | 35-95% | 40-95% | 50-95% |
| Cu | 5-75% | 5-65% | 5-60% | 5-50% |
| C | 0.05-0.15% | 0-0.15% | 0-0.15% | 0-0.15% |
| Cs₂O | 0-0.2% | 0-0.2% | 0.04-0.1% | 0-0.2% |
| Hf | 0.8-1.4% | 0-2.5% | 0-2.5% | 0-2.5% |
| La₂O₃ | 0-2% | 0.3-0.7% | 0-2% | 0-2% |
| Re | 0-40% | 0-40% | 0-40% | 0-40% |
| Ta | 0-50% | 0-50% | 0-50% | 0-50% |
| Y₂O₃ | 0-1% | 0-1% | 0.3-0.5% | 0-1% |
| ZrO₂ | 0-3% | 0-3% | 0-3% | 0-3% |

| | Wt. % | | |
|---|---|---|---|
| Metal | Ex. 126 | Ex. 127 | Ex. 128 |
| W | 55-99% | 60-99% | 70-99% |
| Cu | 1-45% | 1-40% | 1-30% |
| C | 0-0.15% | 0-0.15% | 0-0.15% |
| Cs₂O | 0-0.2% | 0-0.2% | 0-0.2% |
| Hf | 0-2.5% | 0-2.5% | 0-2.5% |
| La₂O₃ | 0-2% | 0-2% | 0-2% |
| Re | 0-40% | 0-40% | 5-40% |
| Ta | 0-50% | 10-50% | 0-50% |
| W | 0-50% | 0-50% | 0-50% |
| Y₂O₃ | 0-1% | 0-1% | 0-1% |
| ZrO₂ | 1.2-1.8% | 0-3% | 0-3% |

| | Wt. % | | |
|---|---|---|---|
| Metal | Ex. 129 | Ex. 130 | Ex. 131 |
| Ti | 55-80% | 65-80% | 70-80% |
| Mo | 20-30% | 20-30% | 20-25% |
| Re | <0.5% | <0.5% | <0.5% |
| Yt | <0.5% | <0.5% | <0.5% |
| Nb | <0.5% | <0.5% | <0.5% |
| Co | <0.5% | <0.5% | <0.5% |
| Cr | <0.5% | <0.5% | <0.5% |
| Zr | <0.5% | <0.5% | <0.5% |
| C | ≤0.15% | ≤0.15% | ≤0.15% |
| O | ≤0.06% | ≤0.06% | ≤0.06% |
| N | ≤20 ppm | ≤20 ppm | ≤20 ppm |
| | Wt. % | | |
| Metal | Ex. 132 | Ex. 133 | Ex. 134 |
| W | 20-99% | 60-99% | 20-80% |
| Re | 1-47.5% | 1-40% | 1-47.5% |
| Mo | 0-47.5% | <0.5% | 1-47.5% |
| | Wt. % | | |
| Metal | Ex. 135 | Ex. 136 | Ex. 137 |
| W | 50.1-99% | 65-99% | 70-99% |
| Re | 1-40% | 1-35% | 1-30% |
| Mo | 0-40% | <0.5% | 1-30% |

| | Wt. % | | |
|---|---|---|---|
| Metal | Ex. 138 | Ex. 139 | Ex. 140 |
| W | 20-49% | 20-49% | 20-49% |
| Re | 1-40% | 1-40% | 1-39% |
| Mo | 20-60% | 30-60% | 40-60% |

| | Wt. % | | |
|---|---|---|---|
| Metal | Ex. 141 | Ex. 142 | Ex. 143 |
| W | 20-40% | 20-35% | 20-30% |
| Re | 1-40% | 10-40% | 25-40% |
| Mo | 0-40% | 10-40% | 25-40% |

| | Wt. % | | |
|---|---|---|---|
| Metal | Ex. 144 | Ex. 145 | Ex. 146 |
| W | 20-99% | 60-99% | 20-80% |
| Re | 1-47.5% | 1-40% | 1-47.5% |
| Mo | 0-47.5% | <0.5% | 1-47.5% |
| Cu | <0.5% | <0.5% | <0.5% |
| C | ≤0.15% | ≤0.15% | ≤0.15% |
| Co | ≤0.002% | ≤0.002% | ≤0.002% |
| Cs₂O | ≤0.2% | ≤0.2% | ≤0.2% |
| Fe | ≤0.02% | ≤0.02% | ≤0.02% |
| H | ≤0.002% | ≤0.002% | ≤0.002% |
| Hf | <0.5% | <0.5% | <0.5% |
| La₂O₃ | <0.5% | <0.5% | <0.5% |
| O | ≤0.06% | ≤0.06% | ≤0.06% |
| Os | <0.5% | <0.5% | <0.5% |
| N | ≤20 ppm | ≤20 ppm | ≤20 ppm |
| Nb | ≤0.01% | ≤0.01% | ≤0.01% |
| Pt | <0.5% | <0.5% | <0.5% |
| S | ≤0.008% | ≤0.008% | ≤0.008% |
| Sn | ≤0.002% | ≤0.002% | ≤0.002% |
| Ta | <0.5% | <0.5% | <0.5% |
| Tc | <0.5% | <0.5% | <0.5% |
| Ti | <0.5% | <0.5% | <0.5% |
| V | <0.5% | <0.5% | <0.5% |
| Y₂O₃ | <0.5% | <0.5% | <0.5% |
| Zr | <0.5% | <0.5% | <0.5% |
| ZrO₂ | <0.5% | <0.5% | <0.5% |
| CNT | 0-10% | 0-10% | <0.5%. |

In Examples 1-146, it will be appreciated that all of the above ranges include any value between the range and any other range that is between the ranges set forth above. Any of the above values that include the <symbol includes the range from 0 to the stated value and all values and ranges therebetween.

In the above refractory metal alloys, the average grain size of the refractory metal alloy can be about 4-20 ASTM, the tensile elongation of the refractory metal alloy can be about 25-50%, the average density of the refractory metal alloy can be at least about 5 gm/cc, the average yield strength of the refractory metal alloy can be about 70-250 (ksi), the average ultimate tensile strength of the refractory metal alloy can be about 80-550 UTS (ksi), and an average Vickers hardness can be 234 DPH to 700 DPH or a Rockwell C hardness of 19-60 at 77° F.; however, this is not required.

In accordance with another and/or alternative non-limiting aspect of the present disclosure, the refractory metal alloy is optionally at least partially formed by a swaging process; however, this is not required. In one non-limiting embodiment, swaging is performed on the refractory metal alloy to at least partially or fully achieve final dimensions of one or more portions of the prosthetic heart valve. The swaging dies can be shaped to fit the final dimension of the prosthetic heart valve; however, this is not required. Where there are undercuts of hollow structures in the prosthetic heart valve (which is not required), a separate piece of metal can be placed in the undercut to at least partially fill the gap. The separate piece of metal (when used) can be designed to be later removed from the undercut; however, this is not required. The swaging operation can be performed on the prosthetic heart valve in the areas to be hardened. For a round or curved portion of a heart valve, the swaging can be rotary. For non-round portion of the prosthetic heart valve, the swaging of the non-round portion of the prosthetic heart valve can be performed by non-rotating swaging dies. The dies can optionally be made to oscillate in radial and/or longitudinal directions instead of or in addition to rotating. The prosthetic heart valve can optionally be swaged in multiple directions in a single operation or in multiple operations to achieve a hardness in desired location and/or direction of the prosthetic heart valve. The swaging temperature for a particular refractory metal alloy can vary. For a refractory metal alloy (e.g., MoRe alloy, ReW alloy, ReCr alloy, etc.), the swaging temperature can be from room temperature (RT) (e.g., 10-27° C. and all values and ranges therebetween) to about 400° C. (e.g., 10-400° C. and all values and ranges therebetween) if the swaging is conducted in air or an oxidizing environment. The swaging temperature can be increased to up to about 1500° C. (e.g., 10-1500° C. and all values and ranges therebetween) if the swaging process is performed in a controlled neutral or non-reducing environment (e.g., inert environment). The swaging process can be conducted by repeatedly hammering the prosthetic heart valve at the location to be hardened at the desired swaging temperature. In one non-limiting embodiment, during the swaging process ions of boron and/or nitrogen are allowed to impinge upon rhenium atoms in the refractory metal alloys that include rhenium to form ReB₂, ReN₂ and/or ReN₃; however, this is not required. It has been found that ReB₂, ReN₂ and/or ReN₃ are ultra-hard compounds. As can be appreciated, other refractory metal alloys that include Re and that are subjected to a swaging process can also form ReB₂, ReN₂ and/or ReN₃. In one non-limiting process, the refractory metal alloy for the prosthetic heart valve can be machined and shaped to at least partially form the prosthetic heart valve when the refractory metal alloy is in a less hardened state; however, this is not required. As such, the raw starting material can be first annealed to soften and then machined into a desired shape. After the refractory metal alloy is shaped, the refractory metal alloy can be re-hardened. The hardening of the refractory metal alloy of the prosthetic heart valve can improve the wear resistance and/or shape retention of the prosthetic heart valve. The refractory metal alloy of the medical generally cannot be re-hardened by annealing, thus a special rehardening processes is required. Such rehardening can be achieved by the swaging process of the present disclosure.

In accordance with another and/or alternative non-limiting aspect of the present disclosure, the refractory metal alloy can optionally be nitrided; however, this is not required. The nitride layer on the refractory metal alloy can function as a lubricating surface during the optional drawing of the refractory metal alloy when partially or fully forming the frame of the prosthetic heart valve. After the refractory metal alloy is nitrided, the refractory metal alloy is typically cleaned; however, this is not required. During the nitride process, the surface of the refractory metal alloy is modified by the presence of nitrogen. The nitriding process can be by gas nitriding, salt bath nitriding, or plasma nitriding. In gas nitriding, the nitrogen diffuses onto the surface of the refractory metal alloy, thereby creating a nitride layer. The thickness and phase constitution of the resulting nitrided layers can be selected and the process optimized for the particular properties required. During gas nitriding, the refractory metal alloy is generally nitrided in the presence of nitrogen gas or a nitrogen gas mixture (e.g., 90-99% vol. % nitrogen and 1-10 vol. % hydrogen, etc.) for at least 10 seconds a temperature of at least about 400° C. (e.g., 400-1000° C. and all values and ranges therebetween). In one non-limiting nitriding process, the refractory metal alloy is heated in the presence of nitrogen or a nitrogen-hydrogen mixture to a temperature of at least 400° C., and generally about 400-800° C. (and all values and ranges therebetween) for at least 10 seconds (e.g., 10 seconds to 60 minutes and all values and ranges therebetween), and generally about 1-30 minutes. In salt bath nitriding, a nitrogen-containing salt such as cyanide salt is used. During the salt bath nitriding, the refractory metal alloy is generally exposed to temperatures of about 520-590° C. In plasma nitriding, the gas used for plasma nitriding is usually pure nitrogen. Plasma nitriding is often coupled with physical vapor deposition (PVD) process; however, this is not required. Plasma nitriding of the refractory metal alloy generally occurs at a temperature of 220-630° C. (and all values and ranges therebetween). The refractory metal alloy can optionally be exposed to argon and/or hydrogen gas prior to the nitriding process to clean and/or preheat the refractory metal alloy. These gases can be optionally used to clean oxide layers and/or solvents from the surface of the refractory metal alloy. During the nitriding process, the refractory metal alloy can optionally be exposed to hydrogen gas to inhibit or prevent the formation of oxides on the surface of the refractory metal alloy. The thickness of the nitrided surface layer is less than about 1 mm. In one non-limiting embodiment, the thickness of the nitrided surface layer is at least about 50 nanometer and less than about 1 mm (and all values and ranges therebetween). In another non-limiting embodiment, the thickness of the nitrided surface layer is at least about 50 nanometer and less than about 0.1 mm. Generally, the weight percent of nitrogen in the nitrided surface layer is 0.0001-5 wt. % nitrogen (and all values and ranges therebetween). In one non-limiting embodiment, the weight percent of nitrogen in the nitrided surface layer is generally less than one of the primary components of the refractory metal alloy, and typically less than each of the two primary components of the refractory metal alloy. For example, when a refractory metal alloy in the form of a MoRe alloy is nitrided, the weight percent of the nitrogen in the nitrided surface layer is less than a weight percent of the molybdenum in the nitrided surface layer. Also, the weight percent of nitrogen in the nitrided surface layer is less than a weight percent of the rhenium in the nitrided surface layer. In one non-limiting composition of the nitrided surface layer on a MoRe alloy (e.g., 40-99 wt. % Mo, 1-40 wt. % Re), the nitrided surface layer comprises 40-99 wt. % molybdenum (and all values and ranges therebetween), 1-40 wt. % rhenium (and all values and ranges therebetween), and 0.0001-5 wt. % nitrogen (and all values and ranges therebetween). In another non-limiting composition of the nitrided surface layer, the nitride surface layer comprises 40-99 wt. % molybdenum, 1-40 wt. % rhenium, and 0.001-1 wt. % nitrogen. As can be appreciated, other refractory metal alloys can be nitrided. For such other refractory metal alloys, the nitride surface layer typically includes 0.001-5 wt. % nitrogen (and all values and ranges therebetween), and the primary constituents of the refractory metal alloy (e.g., metals that constitute at least 5 wt. % of the refractory metal alloy) are present in the nitride surface layer in a greater weight percent than the nitrogen content in the refractory metal alloy. The nitriding process for the refractory metal alloy can be used to increase surface hardness and/or wear resistance of the prosthetic heart valve, and/or to inhibit or prevent discoloration of the refractory metal alloy (e.g., discoloration by oxidation, etc.). For example, the nitriding process can be used to increase the wear resistance of articulation surfaces or surfaces wear on the refractory metal alloy used in the prosthetic heart valve to extend the life of the prosthetic heart valve, and/or to increase the wear life of mating surfaces on the prosthetic heart valve (e.g., polyethylene liners of joint implants like knees, hips, shoulders, etc.), to reduce particulate generation from use of the prosthetic heart valve, and/or to maintain the outer surface appearance of the refractory metal alloy on the prosthetic heart valve.

In accordance with another and/or alternative non-limiting aspect of the present disclosure, the surface of the refractory metal alloy is optionally nitrided prior to at least one drawing step for the refractory metal alloy.

In accordance with another and/or alternative non-limiting aspect of the present disclosure, after the refractory metal alloy has been annealed, the refractory metal alloy is optionally nitrided prior to being drawn.

In accordance with another and/or alternative non-limiting aspect of the present disclosure, the refractory metal alloy the refractory metal alloy is optionally cleaned to remove nitride compounds on the surface of the refractory metal alloy prior to annealing the refractory metal alloy. The nitride compounds can be removed by a variety of steps such as, but not limited to, grit blasting, polishing, etc. After the refractory metal alloy has been annealed, the refractory metal alloy can be again nitrided prior to one or more drawing steps; however, this is not required. As can be appreciated, the complete outer surface of the refractory metal alloy can be nitrided or a portion of the outer surface of the refractory metal alloy can be nitrided.

In accordance with another and/or alternative non-limiting aspect of the present disclosure, the refractory metal alloy can optionally be nitrided only at selected portions of the outer surface of the refractory metal alloy to obtain different surface characteristics of the refractory metal alloy; however, this is not required.

In accordance with another and/or alternative non-limiting aspect of the present disclosure, the final formed refractory metal alloy can optionally include a nitride outer surface.

In accordance with another and/or alternative non-limiting aspect of the present disclosure, the refractory metal alloy, just prior to or after being partially or fully formed into the desired frame of the prosthetic heart valve, can optionally be cleaned, polished, sterilized, nitrided, etc., for final processing of the refractory metal alloy. In one non-limiting embodiment of the disclosure, the refractory metal alloy is optionally electropolished. In one non-limiting aspect of this embodiment, the refractory metal alloy is cleaned prior to being exposed to the polishing solution; however, this is not required. The cleaning process (when used) can be accomplished by a variety of techniques such as, but not limited to, 1) using a solvent (e.g., acetone, methyl alcohol, etc.) and wiping the refractory metal alloy with a Kimwipe or other appropriate towel, and/or 2) at least partially dipping or immersing the refractory metal alloy in a solvent and then ultrasonically cleaning the refractory metal alloy. As can be appreciated, the refractory metal alloy can be cleaned in other or additional ways. In accordance with another and/or alternative non-limiting aspect of this embodiment, the polishing solution can include one or more acids. One non-limiting formulation of the polishing solution includes about 10-80 percent by volume sulfuric acid (and all values and ranges therebetween). As can be appreciated, other polishing solution compositions can be used. In still another and/or alternative non-limiting aspect of this embodiment, about 5-12 volts (and all values and ranges therebetween) are directed to the refractory metal alloy during the electropolishing process; however, other voltage levels can be used. In yet another and/or alternative non-limiting aspect of this embodiment, the refractory metal alloy is rinsed with water and/or a solvent and allowed to dry to remove polishing solution on the refractory metal alloy.

In accordance with another and/or alternative non-limiting aspect of the present disclosure, the use of the refractory metal alloy to partially or fully form the frame of the prosthetic heart valve can be used to increase the strength, hardness, and/or durability of the medical device or portion of the medical device (e.g., frame of the medical device, etc.) compared with stainless steel, chromium-cobalt alloys, or titanium alloys; thus, a lesser quantity of refractory metal alloy can be used in the medical device or portion of the medical device (e.g., frame of the medical device, etc.) to achieve similar strengths compared to medical devices or frames of medical devices formed of different metals. As such, the resulting medical device can be made smaller and less bulky by use of the refractory metal alloy without sacrificing the strength and durability of the medical device. Such a medical device can have a smaller profile, thus can be inserted in smaller areas, openings, and/or passageways. The refractory metal alloy also can increase the radial strength of the medical device. For example, the thickness of the walls of the medical device or portion of the medical device (e.g., frame of the medical device, etc.) and/or the wires used to at least partially form the medical device or portion of the medical device (e.g., frame of the medical device, etc.) can be made thinner and achieve a similar or improved radial strength as compared with thicker walled medical devices formed of stainless steel, titanium alloys, or cobalt and chromium alloys. The refractory metal alloy also can improve stress-strain properties, bendability, and flexibility of the medical device, thus increasing the life of the medical device. For example, the medical device can be used in regions that subject the medical device to bending. Due to the improved physical properties of the medical device from the refractory metal alloy, the medical device has improved resistance to fracturing in such frequent bending environments. In addition or alternatively, the improved bendability and flexibility of the medical device due to the use of the refractory metal alloy enables the medical device to be more easily inserted into various regions of a body. The refractory metal alloy can also reduce the degree of recoil during the crimping and/or expansion of the medical device. For example, the medical device better maintains its crimped form and/or better maintains its expanded form after expansion due to the use of the refractory metal alloy. As such, when the medical device is to be mounted onto a delivery device when the medical device is crimped, the medical device better maintains its smaller profile during the insertion of the medical device into various regions of a body. Also, the medical device better maintains its expanded profile after expansion to facilitate in the success of the medical device in the treatment area. In addition to the improved physical properties of the medical device by use of the refractory metal alloy, the refractory metal alloy has improved radiopaque properties as compared to standard materials such as stainless steel, TiNi alloys, or cobalt-chromium alloy, thus reducing or eliminating the need for using marker materials on the medical device. For instance, the refractory metal alloy is believed to at least about 10-20% more radiopaque than stainless steel, TiNi alloys, or cobalt-chromium alloy.

In accordance with another and/or alternative non-limiting aspect of the present disclosure, the medical device can include, contain and/or be coated with one or more agents that facilitate in the success of the medical device and/or treated area. The term “agent” includes, but is not limited to a substance, pharmaceutical, biologic, veterinary product, drug, and analogs or derivatives otherwise formulated and/or designed to prevent, inhibit and/or treat one or more clinical and/or biological events, and/or to promote healing. Non-limiting examples of clinical events that can be addressed by one or more agents include, but are not limited to, viral, fungus and/or bacterial infection; vascular diseases and/or disorders; digestive diseases and/or disorders; reproductive diseases and/or disorders; lymphatic diseases and/or disorders; cancer; implant rejection; pain; nausea; swelling; arthritis; bone diseases and/or disorders; organ failure; immunity diseases and/or disorders; cholesterol problems; blood diseases and/or disorders; lung diseases and/or disorders; heart diseases and/or disorders; brain diseases and/or disorders; neuralgia diseases and/or disorders; kidney diseases and/or disorders; ulcers; liver diseases and/or disorders; intestinal diseases and/or disorders; gallbladder diseases and/or disorders; pancreatic diseases and/or disorders; psychological disorders; respiratory diseases and/or disorders; gland diseases and/or disorders; skin diseases and/or disorders; hearing diseases and/or disorders; oral diseases and/or disorders; nasal diseases and/or disorders; eye diseases and/or disorders; fatigue; genetic diseases and/or disorders; burns; scarring and/or scars; trauma; weight diseases and/or disorders; addiction diseases and/or disorders; hair loss; cramps; muscle spasms; tissue repair; nerve repair; neural regeneration and/or the like. Non-limiting examples of agents that can be used include, but are not limited to, 5-fluorouracil and/or derivatives thereof; 5-phenylmethimazole and/or derivatives thereof; ACE inhibitors and/or derivatives thereof; acenocoumarol and/or derivatives thereof; acyclovir and/or derivatives thereof; actilyse and/or derivatives thereof; adrenocorticotropic hormone and/or derivatives thereof, adriamycin and/or derivatives thereof, agents that modulate intracellular Ca2+ transport such as L-type (e.g., diltiazem, nifedipine, verapamil, etc.) or T-type Ca2+ channel blockers (e.g., amiloride, etc.); alpha-adrenergic blocking agents and/or derivatives thereof, alteplase and/or derivatives thereof, amino glycosides and/or derivatives thereof (e.g., gentamycin, tobramycin, etc.); angiopeptin and/or derivatives thereof, angiostatic steroid and/or derivatives thereof, angiotensin II receptor antagonists and/or derivatives thereof; anistreplase and/or derivatives thereof, antagonists of vascular epithelial growth factor and/or derivatives thereof; antibiotics; anti-coagulant compounds and/or derivatives thereof, anti-fibrosis compounds and/or derivatives thereof, antifungal compounds and/or derivatives thereof, anti-inflammatory compounds and/or derivatives thereof, anti-invasive factor and/or derivatives thereof; anti-metabolite compounds and/or derivatives thereof (e.g., staurosporin, trichothecenes, and modified diphtheria and ricin toxins, pseudomonas exotoxin, etc.); anti-matrix compounds and/or derivatives thereof (e.g., colchicine, tamoxifen, etc.); anti-microbial agents and/or derivatives thereof; anti-migratory agents and/or derivatives thereof (e.g., caffeic acid derivatives, nilvadipine, etc.); anti-mitotic compounds and/or derivatives thereof, anti-neoplastic compounds and/or derivatives thereof, anti-oxidants and/or derivatives thereof, anti-platelet compounds and/or derivatives thereof, anti-proliferative and/or derivatives thereof, anti-thrombogenic agents and/or derivatives thereof, argatroban and/or derivatives thereof, ap-1 inhibitors and/or derivatives thereof (e.g., for tyrosine kinase, protein kinase C, myosin light chain kinase, Ca2+/calmodulin kinase II, casein kinase II, etc.); aspirin and/or derivatives thereof; azathioprine and/or derivatives thereof; β-estradiol and/or derivatives thereof, β-1-anticollagenase and/or derivatives thereof, calcium channel blockers and/or derivatives thereof, calmodulin antagonists and/or derivatives thereof (e.g., H7, etc.); CAPTOPRIL and/or derivatives thereof, cartilage-derived inhibitor and/or derivatives thereof, ChIMP-3 and/or derivatives thereof, cephalosporin and/or derivatives thereof (e.g., cefadroxil, cefazolin, cefaclor, etc.); chloroquine and/or derivatives thereof, chemotherapeutic compounds and/or derivatives thereof (e.g., 5-fluorouracil, vincristine, vinblastine, cisplatin, doxyrubicin, adriamycin, tamocifen, etc.); chymostatin and/or derivatives thereof, CILAZAPRIL and/or derivatives thereof, clopidigrel and/or derivatives thereof, clotrimazole and/or derivatives thereof, colchicine and/or derivatives thereof, cortisone and/or derivatives thereof, coumadin and/or derivatives thereof, curacin-A and/or derivatives thereof, cyclosporine and/or derivatives thereof, cytochalasin and/or derivatives thereof (e.g., cytochalasin A, cytochalasin B, cytochalasin C, cytochalasin D, cytochalasin E, cytochalasin F, cytochalasin G, cytochalasin H, cytochalasin J, cytochalasin K, cytochalasin L, cytochalasin M, cytochalasin N, cytochalasin O, cytochalasin P, cytochalasin Q, cytochalasin R, cytochalasin S, chaetoglobosin A, chaetoglobosin B, chaetoglobosin C, chaetoglobosin D, chaetoglobosin E, chaetoglobosin F, chaetoglobosin G, chaetoglobosin J, chaetoglobosin K, deoxaphomin, proxiphomin, protophomin, zygosporin D, zygosporin E, zygosporin F, zygosporin G, aspochalasin B, aspochalasin C, aspochalasin D, etc.); cytokines and/or derivatives thereof, desirudin and/or derivatives thereof, dexamethazone and/or derivatives thereof, dipyridamole and/or derivatives thereof, eminase and/or derivatives thereof, endothelin and/or derivatives thereof endothelial growth factor and/or derivatives thereof, epidermal growth factor and/or derivatives thereof, epothilone and/or derivatives thereof, estramustine and/or derivatives thereof, estrogen and/or derivatives thereof, fenoprofen and/or derivatives thereof; fluorouracil and/or derivatives thereof, flucytosine and/or derivatives thereof, forskolin and/or derivatives thereof; ganciclovir and/or derivatives thereof, glucocorticoids and/or derivatives thereof (e.g., dexamethasone, betamethasone, etc.); glycoprotein IIb/IIIa platelet membrane receptor antibody and/or derivatives thereof, GM-CSF and/or derivatives thereof, griseofulvin and/or derivatives thereof, growth factors and/or derivatives thereof (e.g., VEGF; TGF; IGF; PDGF; FGF, etc.); growth hormone and/or derivatives thereof; heparin and/or derivatives thereof, hirudin and/or derivatives thereof, hyaluronate and/or derivatives thereof, hydrocortisone and/or derivatives thereof, ibuprofen and/or derivatives thereof; immunosuppressive agents and/or derivatives thereof (e.g., adrenocorticosteroids, cyclosporine, etc.); indomethacin and/or derivatives thereof; inhibitors of the sodium/calcium antiporter and/or derivatives thereof (e.g., amiloride, etc.); inhibitors of the IP3 receptor and/or derivatives thereof, inhibitors of the sodium/hydrogen antiporter and/or derivatives thereof (e.g., amiloride and derivatives thereof, etc.); insulin and/or derivatives thereof, interferon α-2-macroglobulin and/or derivatives thereof, ketoconazole and/or derivatives thereof, lepirudin and/or derivatives thereof, LISINOPRIL and/or derivatives thereof, LOVASTATIN and/or derivatives thereof, marevan and/or derivatives thereof, mefloquine and/or derivatives thereof, metalloproteinase inhibitors and/or derivatives thereof, methotrexate and/or derivatives thereof; metronidazole and/or derivatives thereof, miconazole and/or derivatives thereof, monoclonal antibodies and/or derivatives thereof, mutamycin and/or derivatives thereof, naproxen and/or derivatives thereof, nitric oxide and/or derivatives thereof, nitroprusside and/or derivatives thereof; nucleic acid analogues and/or derivatives thereof (e.g., peptide nucleic acids, etc.); nystatin and/or derivatives thereof, oligonucleotides and/or derivatives thereof, paclitaxel and/or derivatives thereof, penicillin and/or derivatives thereof, pentamidine isethionate and/or derivatives thereof, phenindione and/or derivatives thereof, phenylbutazone and/or derivatives thereof; phosphodiesterase inhibitors and/or derivatives thereof, plasminogen activator inhibitor-1 and/or derivatives thereof, plasminogen activator inhibitor-2 and/or derivatives thereof, platelet factor 4 and/or derivatives thereof, platelet derived growth factor and/or derivatives thereof, plavix and/or derivatives thereof, POSTMI 75 and/or derivatives thereof, prednisone and/or derivatives thereof; prednisolone and/or derivatives thereof; probucol and/or derivatives thereof; progesterone and/or derivatives thereof; prostacyclin and/or derivatives thereof; prostaglandin inhibitors and/or derivatives thereof, protamine and/or derivatives thereof, protease and/or derivatives thereof, protein kinase inhibitors and/or derivatives thereof (e.g., staurosporin, etc.); quinine and/or derivatives thereof; radioactive agents and/or derivatives thereof (e.g., Cu-64, Ca-67, Cs-131, Ga-68, Zr-89, Ku-97, Tc-99m, Rh-105, Pd-103, Pd-109, In-111, I-123, I-125, I-131, Re-186, Re-188, Au-198, Au-199, Pb-203, At-211, Pb-212, Bi-212, H₃P₃₂O₄, etc.); rapamycin and/or derivatives thereof; receptor antagonists for histamine and/or derivatives thereof, refludan and/or derivatives thereof; retinoic acids and/or derivatives thereof; revasc and/or derivatives thereof, rifamycin and/or derivatives thereof, sense or anti-sense oligonucleotides and/or derivatives thereof (e.g., DNA, RNA, plasmid DNA, plasmid RNA, etc.); seramin and/or derivatives thereof, steroids; seramin and/or derivatives thereof, serotonin and/or derivatives thereof, serotonin blockers and/or derivatives thereof, streptokinase and/or derivatives thereof, sulfasalazine and/or derivatives thereof; sulfonamides and/or derivatives thereof (e.g., sulfamethoxazole, etc.); sulphated chitin derivatives; Sulphated Polysaccharide Peptidoglycan Complex and/or derivatives thereof, TH1 and/or derivatives thereof (e.g., Interleukins-2, -12, and -15, gamma interferon, etc.); thioprotese inhibitors and/or derivatives thereof; taxol and/or derivatives thereof (e.g., taxotere, baccatin, 10-deacetyltaxol, 7-xylosyl-10-deacetyltaxol, cephalomannine, 10-deacetyl-7-epitaxol, 7 epitaxol, 10-deacetylbaccatin III, 10-deacetylcephaolmannine, etc.); ticlid and/or derivatives thereof, ticlopidine and/or derivatives thereof, tick anti-coagulant peptide and/or derivatives thereof, thioprotese inhibitors and/or derivatives thereof, thyroid hormone and/or derivatives thereof, tissue inhibitor of metalloproteinase-1 and/or derivatives thereof, tissue inhibitor of metalloproteinase-2 and/or derivatives thereof, tissue plasma activators; TNF and/or derivatives thereof, tocopherol and/or derivatives thereof, toxins and/or derivatives thereof, tranilast and/or derivatives thereof, transforming growth factors alpha and beta and/or derivatives thereof, trapidil and/or derivatives thereof; triazolopyrimidine and/or derivatives thereof, vapiprost and/or derivatives thereof, vinblastine and/or derivatives thereof; vincristine and/or derivatives thereof; zidovudine and/or derivatives thereof. As can be appreciated, the agent can include one or more derivatives of the above listed compounds and/or other compounds. In one non-limiting embodiment, the agent includes, but is not limited to, trapidil, trapidil derivatives, taxol, taxol derivatives (e.g., taxotere, baccatin, 10-deacetyltaxol, 7-xylosyl-10-deacetyltaxol, cephalomannine, 10-deacetyl-7-epitaxol, 7 epitaxol, 10-deacetylbaccatin III, 10-deacetylcephaolmannine, etc.), cytochalasin, cytochalasin derivatives (e.g., cytochalasin A, cytochalasin B, cytochalasin C, cytochalasin D, cytochalasin E, cytochalasin F, cytochalasin G, cytochalasin H, cytochalasin J, cytochalasin K, cytochalasin L, cytochalasin M, cytochalasin N, cytochalasin O, cytochalasin P, cytochalasin Q, cytochalasin R, cytochalasin S, chaetoglobosin A, chaetoglobosin B, chaetoglobosin C, chaetoglobosin D, chaetoglobosin E, chaetoglobosin F, chaetoglobosin G, chaetoglobosin J, chaetoglobosin K, deoxaphomin, proxiphomin, protophomin, zygosporin D, zygosporin E, zygosporin F, zygosporin G, aspochalasin B, aspochalasin C, aspochalasin D, etc.), paclitaxel, paclitaxel derivatives, rapamycin, rapamycin derivatives, 5-phenylmethimazole, 5-phenylmethimazole derivatives, GM-CSF (granulo-cytemacrophage colony-stimulating-factor), GM-CSF derivatives, statins or HMG-CoA reductase inhibitors forming a class of hypolipidemic agents, combinations, or analogs thereof, or combinations thereof. The type and/or amount of agent included in medical device and/or coated on medical device can vary. When two or more agents are included in and/or coated on medical device, the amount of two or more agents can be the same or different. The type and/or amount of agent included on, in and/or in conjunction with medical device are generally selected to address one or more clinical events.

In accordance with another and/or alternative non-limiting aspect of the present disclosure, the amount of agent included on, in and/or used in conjunction with medical device, when the agent is used, is about 0.01-100 ug per mm² (and all values and ranges wherein between) and/or at least about 0.00001 wt. % of device; however, other amounts can be used. The amount of two of more agents on, in and/or used in conjunction with medical device can be the same or different. The one or more agents can be coated on and/or impregnated in medical device by a variety of mechanisms such as, but not limited to, spraying (e.g., atomizing spray techniques, etc.), flame spray coating, powder deposition, dip coating, flow coating, dip-spin coating, roll coating (direct and reverse), sonication, brushing, plasma deposition, depositing by vapor deposition, MEMS technology, and rotating mold deposition. The amount of two of more agents on, in and/or used in conjunction with medical device, when two one more agents are used, can be the same or different.

In accordance with another and/or alternative non-limiting aspect of the present disclosure, the one or more agents on and/or in the medical device, when used on the medical device, can be released in a controlled manner so the area in question to be treated is provided with the desired dosage of agent over a sustained period of time. As can be appreciated, controlled release of one or more agents on the medical device is not always required and/or desirable. As such, one or more of the agents on and/or in the medical device can be uncontrollably released from the medical device during and/or after insertion of the medical device in the treatment area. It can also be appreciated that one or more agents on and/or in the medical device can be controllably released from the medical device and one or more agents on and/or in the medical device can be uncontrollably released from the medical device. It can also be appreciated that one or more agents on and/or in one region of the medical device can be controllably released from the medical device and one or more agents on and/or in the medical device can be uncontrollably released from another region on the medical device. As such, the medical device can be designed such that 1) all the agent on and/or in the medical device is controllably released, 2) some of the agent on and/or in the medical device is controllably released and some of the agent on the medical device is non-controllably released, or 3) none of the agent on and/or in the medical device is controllably released. The medical device can also be designed such that the rate of release of the one or more agents from the medical device is the same or different. The medical device can also be designed such that the rate of release of the one or more agents from one or more regions on the medical device is the same or different. Non-limiting arrangements that can be used to control the release of one or more agents from the medical device include 1) at least partially coat one or more agents with one or more polymers, 2) at least partially incorporate and/or at least partially encapsulate one or more agents into and/or with one or more polymers, and/or 3) insert one or more agents in pores, passageway, cavities, etc. in the medical device and at least partially coat or cover such pores, passageway, cavities, etc. with one or more polymers. As can be appreciated, other or additional arrangements can be used to control the release of one or more agents from the medical device.

In accordance with another and/or alternative non-limiting aspect of the present disclosure, the one or more polymers used to at least partially control the release of one or more agents from the medical device can be porous or non-porous. The one or more agents can be inserted into and/or applied to one or more surface structures and/or micro-structures on the medical device, and/or be used to at least partially form one or more surface structures and/or micro-structures on the medical device. As such, the one or more agents on the medical device can be 1) coated on one or more surface regions of the medical device, 2) inserted and/or impregnated in one or more surface structures and/or micro-structures, etc. of the medical device, and/or 3) form at least a portion or be included in at least a portion of the structure of the medical device. When the one or more agents are coated on the medical device, the one or more agents can 1) be directly coated on one or more surfaces of the medical device, 2) be mixed with one or more coating polymers or other coating materials and then at least partially coated on one or more surfaces of the medical device, 3) be at least partially coated on the surface of another coating material that has been at least partially coated on the medical device, and/or 4) be at least partially encapsulated between a) a surface or region of the medical device and one or more other coating materials and/or b) two or more other coating materials. As can be appreciated, many other coating arrangements can be additionally or alternatively used. When the one or more agents are inserted and/or impregnated in one or more internal structures, surface structures and/or micro-structures of the medical device, 1) one or more other coating materials can be applied at least partially over the one or more internal structures, surface structures and/or micro-structures of the medical device, and/or 2) one or more polymers can be combined with one or more agents. As such, the one or more agents can be 1) embedded in the structure of the medical device; 2) positioned in one or more internal structures of the medical device; 3) encapsulated between two polymer coatings; 4) encapsulated between the base structure and a polymer coating; and/or 5) mixed in the base structure of the medical device that includes at least one polymer coating. In addition or alternatively, the one or more coating of the one or more polymers on the medical device can include 1) one or more coatings of non-porous polymers; 2) one or more coatings of a combination of one or more porous polymers and one or more non-porous polymers; and/or 3) one or more coating of porous polymer.

In accordance with another and/or alternative non-limiting aspect of the present disclosure, different agents can optionally be located in and/or between different polymer coating layers and/or on and/or the structure of the medical device. As can also be appreciated, many other and/or additional coating combinations and/or configurations can be used. The concentration of one or more agents, the type of polymer, the type and/or shape of internal structures in the medical device and/or the coating thickness of one or more agents can be used to control the release time, the release rate and/or the dosage amount of one or more agents; however, other or additional combinations can be used. As such, the agent and polymer system combination and location on the medical device can be numerous. As can also be appreciated, one or more agents can be deposited on the top surface of the medical device to provide an initial uncontrolled burst effect of the one or more agents prior to 1) the controlled release of the one or more agents through one or more layers of a polymer system that include one or more non-porous polymers and/or 2) the uncontrolled release of the one or more agents through one or more layers of a polymer system. The one or more agents and/or polymers can be coated on the medical device by a variety of mechanisms such as, but not limited to, spraying (e.g., atomizing spray techniques, etc.), dip coating, roll coating, sonication, brushing, plasma deposition, and/or depositing by vapor deposition.

In accordance with another and/or alternative non-limiting aspect of the present disclosure, the thickness of each polymer layer and/or layer of agent is generally at least about 0.01 μm and is generally less than about 150 μm (e.g., 0.01-149.9999 μm and all values and ranges therebetween). In one non-limiting embodiment, the thickness of a polymer layer and/or layer of agent is about 0.02-75 μm, more particularly about 0.05-50 μm, and even more particularly about 1-30 μm. As can be appreciated, other thicknesses can be used.

In accordance with another and/or alternative non-limiting aspect of the present disclosure, a variety of polymers can be coated on the medical device and/or be used to form at least a portion of the medical device. When one or more layers of polymer are coated onto at least a portion of the medical device, the one or more coatings can be applied by a variety of techniques such as, but not limited to, vapor deposition and/or plasma deposition, spraying, dip-coating, roll coating, sonication, atomization, brushing and/or the like; however, other or additional coating techniques can be used. The one or more polymers that can be coated on the medical device and/or used to at least partially form the medical device can be polymers that are considered to be biodegradable, bioresorbable, or bioerodable; polymers that are considered to be biostable; and/or polymers that can be made to be biodegradable and/or bioresorbable with modification. Non-limiting examples of polymers that are considered to be biodegradable, bioresorbable, or bioerodable include, but are not limited to, aliphatic polyesters; poly(glycolic acid) and/or copolymers thereof (e.g., poly(glycolide trimethylene carbonate); poly(caprolactone glycolide)); poly(lactic acid) and/or isomers thereof (e.g., poly-L(lactic acid) and/or poly-D Lactic acid) and/or copolymers thereof (e.g. DL-PLA), with and without additives (e.g. calcium phosphate glass), and/or other copolymers (e.g. poly(caprolactone lactide), poly(lactide glycolide), poly(lactic acid ethylene glycol)); poly(ethylene glycol); poly(ethylene glycol) diacrylate; poly(lactide); polyalkylene succinate; polybutylene diglycolate; polyhydroxybutyrate (PHB); polyhydroxyvalerate (PHV); polyhydroxybutyrate/polyhydroxyvalerate copolymer (PHB/PHV); poly(hydroxybutyrate-co-valerate); polyhydroxyalkaoates (PHA); polycaprolactone; poly(caprolactone-polyethylene glycol) copolymer; poly(valerolactone); polyanhydrides; poly(orthoesters) and/or blends with polyanhydrides; poly(anhydride-co-imide); polycarbonates (aliphatic); poly(hydroxyl-esters); polydioxanone; polyanhydrides; polyanhydride esters; polycyanoacrylates; poly(alkyl 2-cyanoacrylates); poly(amino acids); poly(phosphazenes); poly(propylene fumarate); poly(propylene fumarate-co-ethylene glycol); poly(fumarate anhydrides); fibrinogen; fibrin; gelatin; cellulose and/or cellulose derivatives and/or cellulosic polymers (e.g., cellulose acetate, cellulose acetate butyrate, cellulose butyrate, cellulose ethers, cellulose nitrate, cellulose propionate, cellophane); chitosan and/or chitosan derivatives (e.g., chitosan NOCC, chitosan NOOC-G); alginate; polysaccharides; starch; amylase; collagen; polycarboxylic acids; poly(ethyl ester-co-carboxylate carbonate) (and/or other tyrosine derived polycarbonates); poly(iminocarbonate); poly(BPA-iminocarbonate); poly(trimethylene carbonate); poly(iminocarbonate-amide) copolymers and/or other pseudo-poly(amino acids); poly(ethylene glycol); poly(ethylene oxide); poly(ethylene oxide)/poly(butylene terephthalate) copolymer; poly(epsilon-caprolactone-dimethyltrimethylene carbonate); poly(ester amide); poly(amino acids) and conventional synthetic polymers thereof, poly(alkylene oxalates); poly(alkylcarbonate); poly(adipic anhydride); nylon copolyamides; NO-carboxymethyl chitosan NOCC); carboxymethyl cellulose; copoly(ether-esters) (e.g., PEO/PLA dextrans); polyketals; biodegradable polyethers; biodegradable polyesters; polydihydropyrans; polydepsipeptides; polyarylates (L-tyrosine-derived) and/or free acid polyarylates; polyamides (e.g., nylon 6-6, polycaprolactam); poly(propylene fumarate-co-ethylene glycol) (e.g., fumarate anhydrides); hyaluronates; poly-p-dioxanone; polypeptides and proteins; polyphosphoester; polyphosphoester urethane; polysaccharides; pseudo-poly(amino acids); starch; terpolymer; (copolymers of glycolide, lactide, or dimethyltrimethylene carbonate); rayon; rayon triacetate; latex; and/pr copolymers, blends, and/or composites of above. Non-limiting examples of polymers that considered to be biostable include, but are not limited to, parylene; parylene c; parylene f, parylene n; parylene derivatives; maleic anyhydride polymers; phosphorylcholine; poly n-butyl methacrylate (PBMA); polyethylene-co-vinyl acetate (PEVA); PBMA/PEVA blend or copolymer; polytetrafluoroethene (Teflon®) and derivatives; poly-paraphenylene terephthalamide (Kevlar®); poly(ether ketone) (PEEK); poly(styrene-b-isobutylene-b-styrene) (Translute™); tetramethyldisiloxane (side chain or copolymer); polyimides polysulfides; poly(ethylene terephthalate); poly(methyl methacrylate); poly(ethylene-co-methyl methacrylate); styrene-ethylene/butylene-styrene block copolymers; ABS; SAN; acrylic polymers and/or copolymers (e.g., n-butyl-acrylate, n-butyl methacrylate, 2-ethylhexyl acrylate, lauryl-acrylate, 2-hydroxy-propyl acrylate, polyhydroxyethyl, methacrylate/methylmethacrylate copolymers); glycosaminoglycans; alkyd resins; elastin; polyether sulfones; epoxy resin; poly(oxymethylene); polyolefins; polymers of silicone; polymers of methane; polyisobutylene; ethylene-alphaolefin copolymers; polyethylene; polyacrylonitrile; fluorosilicones; poly(propylene oxide); polyvinyl aromatics (e.g. polystyrene); poly(vinyl ethers) (e.g. polyvinyl methyl ether); poly(vinyl ketones); poly(vinylidene halides) (e.g. polyvinylidene fluoride, polyvinylidene chloride); poly(vinylpyrolidone); poly(vinylpyrolidone)/vinyl acetate copolymer; polyvinylpridine prolastin or silk-elastin polymers (SELP); silicone; silicone rubber; polyurethanes (polycarbonate polyurethanes, silicone urethane polymer) (e.g., chronoflex varieties, bionate varieties); vinyl halide polymers and/or copolymers (e.g. polyvinyl chloride); polyacrylic acid; ethylene acrylic acid copolymer; ethylene vinyl acetate copolymer; polyvinyl alcohol; poly(hydroxyl alkylmethacrylate); polyvinyl esters (e.g. polyvinyl acetate); and/or copolymers, blends, and/or composites of above. Non-limiting examples of polymers that can be made to be biodegradable and/or bioresorbable with modification include, but are not limited to, hyaluronic acid (hyanluron); polycarbonates; polyorthocarbonates; copolymers of vinyl monomers; polyacetals; biodegradable polyurethanes; polyacrylamide; polyisocyanates; polyamide; and/or copolymers, blends, and/or composites of above. As can be appreciated, other and/or additional polymers and/or derivatives of one or more of the above listed polymers can be used. The one or more polymers can be coated on the medical device by a variety of mechanisms such as, but not limited to, spraying (e.g., atomizing spray techniques, etc.), dip coating, roll coating, sonication, brushing, plasma deposition, and/or depositing by vapor deposition. In one non-limiting embodiment, the medical device includes and/or is coated with parylene, PLGA, POE, PGA, PLLA, PAA, PEG, chitosan and/or derivatives of one or more of these polymers. In another and/or alternative non-limiting embodiment, the medical device includes and/or is coated with a non-porous polymer that includes, but is not limited to, polyamide, Parylene C, Parylene N and/or a parylene derivative. In still another and/or alternative non-limiting embodiment, the medical device includes and/or is coated with poly (ethylene oxide), poly(ethylene glycol), and poly(propylene oxide), polymers of silicone, methane, tetrafluoroethylene (including TEFLON™ brand polymers), tetramethyldisiloxane, and the like.

In accordance with another and/or alternative non-limiting aspect of the present disclosure, the medical device, when including and/or is coated with one or more agents, can include and/or can be coated with one or more agents that are the same or different in different regions of the medical device and/or have differing amounts and/or concentrations in differing regions of the medical device. For instance, the medical device can be 1) coated with and/or include one or more biologicals on at least one portion of the medical device and at least another portion of the medical device is not coated with and/or includes agent, 2) coated with and/or include one or more biologicals on at least one portion of the medical device that is different from one or more biologicals on at least another portion of the medical device, and/or 3) coated with and/or include one or more biologicals at a concentration on at least one portion of the medical device that is different from the concentration of one or more biologicals on at least another portion of the medical device; etc.

In accordance with another and/or alternative non-limiting aspect of the present disclosure, one or more portions of the medical device can optionally 1) include the same or different agents, 2) include the same or different amount of one or more agents, 3) include the same or different polymer coatings, 4) include the same or different coating thicknesses of one or more polymer coatings, 5) have one or more portions of the medical device controllably release and/or uncontrollably release one or more agents, and/or 6) have one or more portions of the medical device controllably release one or more agents and one or more portions of the medical device uncontrollably release one or more agents.

In accordance with another and/or alternative non-limiting aspect of the present disclosure, one or more surfaces of the medical device can optionally be treated to achieve the desired coating properties of the one or more agents and one or more polymers coated on the medical device. Such surface treatment techniques include, but are not limited to, cleaning, buffing, smoothing, nitriding, annealing, swaging, cold working, etching (chemical etching, plasma etching, etc.), etc. As can be appreciated, other or additional surface treatment processes can be used prior to the coating of one or more agents and/or polymers on the surface of the medical device. Once one or more surface regions of the medical device been treated, one or more coatings of polymer and/or agent can be applied to one or more regions of the medical device. The one or more layers of agent can be applied to the medical device by a variety of techniques (e.g., dipping, rolling, brushing, spraying, particle atomization, etc.). One non-limiting coating technique is by an ultrasonic mist coating process wherein ultrasonic waves are used to break up the droplet of agent and form a mist of very fine droplets. These fine droplets have an average droplet diameter of about 0.1-3 microns. The fine droplet mist facilitates in the formation of a uniform coating thickness and can increase the coverage area on the medical device.

In accordance with another and/or alternative non-limiting aspect of the present disclosure, the medical device can optionally include a marker material that facilitates enabling the medical device to be properly positioned in a body passageway (e.g., blood vessel, heart valve, etc.). The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.); and/or other types of electromagnetic waves (e.g., microwaves, visible light, infrared waves, ultraviolet waves, etc.). In one non-limiting embodiment, the marker material is visible to x-rays (i.e., radiopaque). The marker material can form all or a portion of the medical device and/or be coated on one or more portions (flaring portion and/or body portion, at ends of medical device, at or near transition of body portion and flaring section, etc.) of the medical device. The location of the marker material can be on one or multiple locations on the medical device. The size of the one or more regions including the marker material can be the same or different. The marker material can be spaced at defined distances from one another to form ruler-like markings on the medical device to facilitate in the positioning of the medical device in a body passageway. The marker material can be a rigid or flexible material. The marker material can be a biostable or biodegradable material. When the marker material is a rigid material, the marker material is typically formed of a metal material (e.g., metal band, metal plating, etc.); however, other or additional materials can be used. The metal, which at least partially forms the medical device, can function as a marker material; however, this is not required. When the marker material is a flexible material, the marker material typically is formed of one or more polymers that are marker materials in-of-themselves and/or include one or more metal powders and/or metal compounds. In one non-limiting embodiment, the flexible marker material includes one or more metal powders in combinations with parylene, PLGA, POE, PGA, PLLA, PAA, PEG, chitosan and/or derivatives of one or more of these polymers. In another and/or alternative non-limiting embodiment, the flexible marker material includes one or more metals and/or metal powders of aluminum, barium, bismuth, cobalt, copper, chromium, gold, iron, stainless steel, titanium, vanadium, nickel, zirconium, niobium, lead, molybdenum, platinum, yttrium, calcium, rare earth metals, rhenium, zinc, silver, depleted radioactive elements, tantalum and/or tungsten; and/or compounds thereof. The marker material can be coated with a polymer protective material; however, this is not required. When the marker material is coated with a polymer protective material, the polymer coating can be used to 1) at least partially insulate the marker material from body fluids, 2) facilitate in retaining the marker material on the medical device, 3) at least partially shield the marker material from damage during a medical procedure and/or 4) provide a desired surface profile on the medical device. As can be appreciated, the polymer coating can have other or additional uses. The polymer protective coating can be a biostable polymer or a biodegradable polymer (e.g., degrades and/or is absorbed). The coating thickness of the protective coating polymer material, when used, is typically less than about 300 microns (e.g., 0.001-299.999 microns and all values and ranges therebetween); however, other thickness can be used. In one non-limiting embodiment, the protective coating materials include parylene, PLGA, POE, PGA, PLLA, PAA, PEG, chitosan and/or derivatives of one or more of these polymers.

In accordance with another and/or alternative non-limiting aspect of the present disclosure, the medical device or one or more regions of the medical device can optionally be constructed by use of one or more microelectromechanical manufacturing (MEMS) techniques (e.g., micro-machining, laser micro-machining, laser micro-machining, micro-molding, 3D printing, etc.); however, other or additional manufacturing techniques can be used.

In accordance with another and/or alternative non-limiting aspect of the present disclosure, the medical device can optionally include one or more surface structures (e.g., pore, channel, pit, rib, slot, notch, bump, teeth, needle, well, hole, groove, etc.). These structures can be at least partially formed by MEMS (e.g., micro-machining, etc.) technology and/or other types of technology (e.g., 3D printing, etc.).

In accordance with another and/or alternative non-limiting aspect of the present disclosure, the medical device can optionally include one or more micro-structures (e.g., micro-needle, micro-pore, micro-cylinder, micro-cone, micro-pyramid, micro-tube, micro-parallelopiped, micro-prism, micro-hemisphere, teeth, rib, ridge, ratchet, hinge, zipper, zip-tie-like structure, etc.) on the surface of the medical device. As defined herein, a “micro-structure” is a structure having at least one dimension (e.g., average width, average diameter, average height, average length, average depth, etc.) that is no more than about 2 mm, and typically no more than about 1 mm. As can be appreciated, when the medical device includes one or more surface structures, 1) all the surface structures can be micro-structures, 2) all the surface structures can be non-micro-structures, or 3) a portion of the surface structures can be micro-structures and a portion can be non-micro-structures. Non-limiting examples of structures that can be formed on the medical device are illustrated in United States Patent Publication Nos. 2004/0093076 and 2004/0093077, which are incorporated herein by reference. Typically, the micro-structures (when formed) extend from or into the outer surface no more than about 400 microns (0.01-400 microns and all values and ranges therebetween), and more typically less than about 300 microns, and more typically about 15-250 microns; however, other sizes can be used. The micro-structures can be clustered together or disbursed throughout the surface of the medical device. Similar shaped and/or sized micro-structures and/or surface structures can be used, or different shaped and/or sized micro-structures can be used. When one or more surface structures and/or micro-structures are designed to extend from the surface of the medical device, the one or more surface structures and/or micro-structures can be formed in the extended position and/or be designed to extend from the medical device during and/or after deployment of the medical device in a treatment area. The micro-structures and/or surface structures can be designed to contain and/or be fluidly connected to a passageway, cavity, etc.; however, this is not required. The one or more surface structures and/or micro-structures can be used to engage and/or penetrate surrounding tissue or organs once the medical device has been positioned on and/or in a patient; however, this is not required. The one or more surface structures and/or micro-structures can be used to facilitate in forming maintaining a shape of a medical device. In one non-limiting embodiment, the one or more surface structures and/or micro-structures can be at least partially formed of an agent and/or be formed of a polymer. One or more of the surface structures and/or micro-structures can include one or more internal passageways that can include one or more materials (e.g., agent, polymer, etc.); however, this is not required. The one or more surface structures and/or micro-structures can be formed by a variety of processes (e.g., machining, chemical modifications, chemical reactions, MEMS (e.g., micro-machining, etc.), etching, laser cutting, 3D printing, photo-etching, etc.). The one or more coatings and/or one or more surface structures and/or micro-structures of the medical device can be used for a variety of purposes such as, but not limited to, 1) increasing the bonding and/or adhesion of one or more agents, adhesives, marker materials and/or polymers to the medical device, 2) changing the appearance or surface characteristics of the medical device, and/or 3) controlling the release rate of one or more agents. The one or more micro-structures and/or surface structures can be biostable, biodegradable, etc. One or more regions of the medical device that are at least partially formed by MEMS techniques can be biostable, biodegradable, etc. The medical device or one or more regions of the medical device can be at least partially covered and/or filled with a protective material to at least partially protect one or more regions of the medical device, and/or one or more micro-structures and/or surface structures on the medical device from damage. One or more regions of the medical device, and/or one or more micro-structures and/or surface structures on the medical device can be damaged when the medical device is 1) packaged and/or stored, 2) unpackaged, 3) connected to and/or other secured and/or placed on another medical device, 4) inserted into a treatment area, and/or 5) handled by a user. As can be appreciated, the medical device can be damaged in other or additional ways. The protective material can be used to protect the medical device and/or one or more micro-structures and/or surface structures from such damage. The protective material can include one or more polymers previously identified above. The protective material can be 1) biostable and/or biodegradable and/or 2) porous and/or non-porous. In another and/or additional non-limiting design, the protective material includes, but is not limited to, sugar (e.g., glucose, fructose, sucrose, etc.), carbohydrate compound, salt (e.g., NaCl, etc.), parylene, PLGA, POE, PGA, PLLA, PAA, PEG, chitosan and/or derivatives of one or more of these materials; however, other and/or additional materials can be used. In still another and/or additional non-limiting design, the thickness of the protective material is generally less than about 300 microns (e.g., 0.01 microns to 299.9999 microns and all values and ranges therebetween), and typically less than about 150 microns; however, other thicknesses can be used. The protective material can be coated by one or more mechanisms previously described herein.

In accordance with another and/or alternative non-limiting aspect of the present disclosure, the medical device can optionally be an expandable device that can be expanded by use of some other device (e.g., balloon, etc.).

In accordance with another and/or alternative non-limiting aspect of the present disclosure, the medical device can optionally be fabricated from a material having no or substantially no shape-memory characteristics.

In accordance with another and/or alternative non-limiting aspect of the present disclosure, there is optionally provided a near net process for a frame and/or other metal component of the medical device. In one non-limiting embodiment of the disclosure, there is provided a method of powder pressing materials and optionally increasing the strength post-sintering by imparting additional cold work. In one non-limiting embodiment, the green part is pressed and then sintered. Thereafter, the sintered part is again pressed to increase its mechanical strength by imparting cold work into the pressed and sintered part. Generally, the temperature during the pressing process after the sintering process is 20-100° C. (and all values and ranges therebetween), typically 20-80° C., and more typically 20-40° C. As defined herein, cold working occurs at a temperature of no more than 150° C. (e.g., 10-150° C. and all values and ranges therebetween). The change in the shape of the repressed post-sintered part needs to be determined so the final part (pressed, sintered, and re-pressed) meets the dimensional requirements of the final formed part. For a Mo47.5Re alloy, MoRe alloy, ReW alloy, molybdenum alloy, tungsten alloy, rhenium alloy, other refractory metal alloys, a prepress pressure of 1-300 tsi (1 ton per square inch) (and all values and ranges therebetween) can be used followed by a sintering process of at least 1600° C. (e.g., 1600-2600° C. and all values and ranges therebetween) and a post sintering press at a pressure of 1-300 tsi (and all values and ranges therebetween) at a temperature of at least 20° C. (e.g., 20-100° C. and all values and ranges therebetween; 20-40° C., etc.). There is also provided an optional process of increasing the mechanical strength of a pressed metal part by repressing the post-sintered part to add additional cold work into the material, thereby increasing its mechanical strength. There is also provided an optional process of powder pressing to a near net or final part using metal powder. In one non-limiting embodiment, the metal powder used to form the near net or final part includes a minimum of 40 wt. % rhenium and at least 25 wt. % molybdenum, and remainder can optionally include one or more elements of tungsten, tantalum, chromium, niobium, zirconium, iridium, titanium, bismuth, and yttrium. In another non-limiting embodiment, the metal powder used to form the near net or final part includes 20-80 wt. % rhenium (and all values and ranges therebetween), 20-80 wt. % molybdenum (and all values and ranges therebetween), and optionally one or more elements of tungsten, tantalum, chromium, niobium, zirconium, iridium, titanium, bismuth, and yttrium. In another non-limiting embodiment, the metal powder used to form the near net or final part includes tungsten (20-60 wt. % and all values and ranges therebetween), rhenium (20-80 wt. % and all values and ranges therebetween) and one or more other elements 0-5 wt. % (and all values and ranges therebetween). In another non-limiting embodiment, the metal powder used to form the near net or final part includes tungsten (20-80 wt. % and all values and ranges therebetween), rhenium (20-80 wt. % and all values and ranges therebetween), molybdenum (0.01-15 wt. % and all values and ranges therebetween), and one or more other elements 0-5 wt. % (and all values and ranges therebetween). In another non-limiting embodiment, the metal powder used to form the near net or final part includes tungsten (20-80 wt. % and all values and ranges therebetween), copper (1-30 wt. % and all values and ranges therebetween), and one or more other elements 0-5 wt. % (and all values and ranges therebetween). In another non-limiting embodiment, the metal powder used to form the near net or final part includes 35-65 wt. % rhenium (and all values and ranges therebetween), and two or more elements of tungsten, tantalum, molybdenum, chromium, niobium, zirconium, iridium, titanium, bismuth, and yttrium. In another non-limiting embodiment, the metal powder used to form the near net or final part includes 35-65 wt. % rhenium (and all values and ranges therebetween) molybdenum powder, and 11-41 wt. % (and all values and ranges therebetween) a combination of chromium powder and optionally a powder of one or more metals selected from the group consisting of bismuth, tungsten, tantalum, molybdenum, chromium, niobium, zirconium, iridium, niobium, tantalum, titanium, bismuth, and yttrium. In another non-limiting embodiment, the metal powder used to form the near net or final part includes 35-65 wt. % rhenium (and all values and ranges therebetween), and chromium, and 0.1-25 wt. % (and all values and ranges therebetween) and one or more elements of molybdenum, bismuth, niobium, tungsten, tantalum, titanium, vanadium, tungsten, manganese, zirconium, technetium, ruthenium, rhodium, hafnium, osmium, copper, iridium, and yttrium. In another non-limiting embodiment, the metal powder used to form the near net or final part includes 25-95 wt. % rhenium (and all values and ranges therebetween), and one or more of calcium, carbon, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, zinc, zirconium, and/or alloys of one or more of such components.

In accordance with another and/or alternative non-limiting aspect of the present disclosure, there is optionally provided a press of near net or finished part composite. The process of pressing metals into near net of finished parts is well established; however, pressing a composite structure formed of metal powder and polymer for purposes of making complex part geometries and foam like structures is new. Similarly, using a pressing process to impart particular biologic substances into the metal matrix is also new. In one non-limiting embodiment, there is provided a process of creating a metal part with pre-defined voids to create a trabecular or foam structure composed of mixing a metal and polymer powder, and then pressing the powder into a finished part or semi-finished green part, and then sintering the part under which conditions the polymer leaves the metal behind through a process of thermal degradation of the polymer. The resulting part has a porosity associated with the size of the polymer particles as well as the homogeneity of the mixture upon pressing prior to sintering. In another non-limiting embodiment, there is provided a process by which a residual of the polymer is left behind after thermal degradation (on the metal substrate) and the polymer residual has some desired biological affect (e.g., masking the metal from the body by encapsulation, promotion of cellular attachment and growth). The polymer and metal powders can be of varying sizes to create a multiplied of voids—some large, creating a pathway for cellular growth, and some small, creating a ruff surface to promote cellular attachment.

In accordance with another and/or alternative non-limiting aspect of the present disclosure, the polymer can optionally be uniformly or non-uniformly dispersed with the metal powder. For example, if the final formed part is to have a uniform density and pore structure, the polymer material is uniformly dispersed with the metal powder prior to consolidating and pressing the polymer and metal powders together and then subsequently sintering together the metal powder to form the metal part or medical device or portion of the medical device (e.g., frame of the medical device, etc.). Alternatively, if the formed metal part or medical device or portion of the medical device (e.g., frame of the medical device, etc.) is to have one or more channels, passageways, and/or voids on the outer surface and/or within the formed part or medical device or portion of the medical device (e.g., frame of the medical device, etc.), at least a portion of the polymer is not uniformly distributed with the metal powder, but instead is concentrated or forms all of the region that is to be the one or more channels, passageways, and/or voids on the outer surface and/or within the formed part or medical device or portion of the medical device (e.g., frame of the medical device, etc.) such that when the polymer and metal powder is sintered, some or all of the polymer is degraded and removed from the part or medical device or portion of the medical device (e.g., frame of the medical device, etc.) thereby forming such one or more channels, passageways, and/or voids on the outer surface and/or within the formed metal part or medical device or portion of the medical device (e.g., frame of the medical device, etc.). As such, the use of a polymer in combination with metal powder and subsequent pressing and sintering can be used to form novel and customized shapes for medical device or portion of the medical device (e.g., frame of the medical device, etc.) or the near net form of the medical device or portion of the medical device (e.g., frame of the medical device, etc.). Generally, the polymer constitutes about 0.1-70 vol. % (and all values and ranges therebetween) of the consolidated and pressed material prior to the sintering step, typically the polymer constitutes about 1-60 vol. % of the consolidated and pressed material prior to the sintering step, more typically the polymer constitutes about 2-50 vol. % of the consolidated and pressed material prior to the sintering step, and even more typically the polymer constitutes about 2-45 vol. % of the consolidated and pressed material prior to the sintering step. As such, if the polymer constitutes about 5 vol. % of the consolidated and pressed material prior to the sintering step, if after the sintering step at least 95% (e.g., 95-100% and all values and ranges therebetween) of the polymer is degraded and removed from the part or medical device or portion of the medical device (e.g., frame of the medical device, etc.), then the part could include up to about 5 vol. % cavities and/or passageways in the medical device or portion of the medical device (e.g., frame of the medical device, etc.).

The type of polymer and the type of metal powder is non-limiting. The polymer and metal powders can be of varying sizes to create multiple voids/passageways/channels which can be used to create a pathway for cellular growth, create a ruff surface to promote cellular attachment, have a biological agent inserted into one or more of the voids/passageways/channels, have biological material inserted into one or more of the voids/passageways/channels, etc. In one non-limiting embodiment, the average particle size of the polymer is greater than the average particle size of the metal powder prior to sintering.

In another non-limiting aspect of the present disclosure, after the sintering process, at least 95 vol. % (95%-100% and all values and ranges therebetween) of the polymer is thermally degraded and/or removed from the sintered material, typically at least 99 vol. % of the polymer is thermally degraded and/or removed from the sintered material, more typically at least 99.5 vol. % of the polymer is thermally degraded and/or removed from the sintered material, still even more typically at least 99.9 vol. % of the polymer is thermally degraded and/or removed from the sintered material, and even still more typically at least 99.95 vol. % of the polymer is thermally degraded and/or removed from the sintered material. The resulting part or medical device or portion of the medical device (e.g., frame of the medical device, etc.) has a porosity associated with the size of the polymer particles as well as the homogeneity of the mixture upon pressing prior to sintering.

In another non-limiting aspect of the present disclosure, after the sintering process, some of the polymer may optionally remain in the sintered metal part or medical device or portion of the medical device (e.g., frame of the medical device, etc.). The remaining polymer in the sintered part or the medical device or portion of the medical device (e.g., frame of the medical device, etc.) can optionally have some desired biological affect (e.g., masking the metal from the body by encapsulation, promotion of cellular attachment and growth, etc.). Any remaining polymer can optionally include one or more biological agents that remain active after the sintering process. In one non-limiting embodiment, when polymer is designed to remain in the sintered part, after the sintering process, about 5-99.9 vol. % (and all values and ranges therebetween) of the polymer is thermally degraded and/or removed from the sintered material, typically about 10-95 vol. % of the polymer is thermally degraded and removed from the sintered material, and more typically about 10-80 vol. % of the polymer is thermally degraded and removed from the sintered material.

In accordance with another and/or alternative non-limiting aspect of the present disclosure, the refractory metal alloy used to at least partially form the medical device or portion of the medical device (e.g., frame of the medical device, etc.) is initially formed into a near net part, blank, a rod, a tube, etc., and then finished into final form by one or more finishing processes (e.g., centerless grinding, turning, electropolishing, drawing process, grinding, laser cutting, shaving, polishing, EDM cutting, micro-machining, laser micro-machining, micro-molding, machining, drilling (e.g., gun drilling, etc.), 3D printing, cold wording, swaging, cleaning, buffing, smoothing, nitriding, annealing, plug drawing, etching (chemical etching, plasma etching, etc.), chemical modifications, chemical reactions, photo-etching, chemical coatings, etc.).

In accordance with another and/or alternative non-limiting aspect of the present disclosure, the refractory metal alloy near net part, blank, rod, tube, etc., can be formed by various techniques such as, but not limited to, 1) melting the refractory metal alloy and/or metals that form the refractory metal alloy (e.g., vacuum arc melting, etc.) and then extruding and/or casting the refractory metal alloy into a near net part, blank, rod, tube, etc., 2) melting the refractory metal alloy and/or metals that form the refractory metal alloy, forming a metal strip and then rolling and welding the strip into a near net part, blank, rod, tube, etc., 3) consolidating (pressing, pressing and sintering, etc.) the metal powder of the refractory metal alloy and/or metal powder of metals that form the refractory metal alloy into a near net part, blank, rod, tube, etc., and/or 4) 3D print the metal alloy into a near net part, blank, rod, tube, etc. When the refractory metal alloy is formed into a blank, the shape and size of the blank is non-limiting. When the refractory metal alloy is formed into a rod or tube, the rod or tube generally has a length of about 48 inches or less (e.g., 0.1-48 inches and all values and ranges therebetween); however, longer lengths can be formed. In one non-limiting arrangement, the length of the rod or tube is about 8-20 inches. The average outer diameter of the rod or tube is generally less than about 2 inches (i.e., less than about 3.14 sq. in. cross-sectional area), more typically less than about 1 inch outer diameter, and even more typically no more than about 0.5 inch outer diameter; however, larger rod or tube diameter sizes can be formed. In one non-limiting configuration for a tube, the tube has an inner diameter of about 0.31 inch plus or minus about 0.002 inch and an outer diameter of about 0.5 inch plus or minus about 0.002 inch. The wall thickness of the tube is about 0.095 inch plus or minus about 0.002 inch. As can be appreciated, this is just one example of many different sized tubes that can be formed. In one non-limiting process, the near net frame of the medical device or portion of the medical device (e.g., frame of the medical device, etc.), blank, rod, tube, etc. In one non-limiting process, the near net medical device or portion of the medical device (e.g., frame of the medical device, etc.), blank, rod, tube, etc., can be formed from one or more ingots of metal or refractory metal alloy. In one non-limiting process, an arc melting process (e.g., vacuum arc melting process, etc.) can be used to form the near net medical device or portion of the medical device (e.g., frame of the medical device, etc.), blank, rod, tube, etc. In another non-limiting process, rhenium powder and tungsten powder and optionally molybdenum powder can be placed in a crucible (e.g., silica crucible, etc.) and heated under a controlled atmosphere (e.g., vacuum environment, carbon monoxide environment, hydrogen and argon environment, helium, argon, etc.) by an induction melting furnace to form the near net medical device or portion of the medical device (e.g., frame of the medical device, etc.), blank, rod, tube, etc. As can be appreciated, other metal particles can be used to form other refractory metal alloys (e.g., molybdenum alloys, rhenium alloys, MoRe alloys, MoReCr alloys, FeCrMoCB alloys, WCu alloys, WRe alloys, ReCr alloys, MoReTa alloy, MoReTi alloy, ReCr alloy, W alloy, Ta alloy, Nb alloy, etc.) by various processes such as melting, sintering, particle compression plus heat, etc. It can be appreciated that other or additional processes can be used to form the refractory metal alloy. When a tube of refractory metal alloy is to be formed, a close-fitting rod can be used during the extrusion process to form the tube; however, this is not required. In another and/or additional non-limiting process, the tube of the refractory metal alloy can be formed from a strip or sheet of refractory metal alloy. The strip or sheet of refractory metal alloy can be formed into a tube by rolling the edges of the sheet or strip and then welding together the edges of the sheet or strip. The welding of the edges of the sheet or strip can be accomplished in several ways such as, but not limited to, a) holding the edges together and then e-beam welding the edges together in a vacuum, b) positioning a thin strip of refractory metal alloy above and/or below the edges of the rolled strip or sheet to be welded, then welding the one or more strips along the rolled strip or sheet edges, and then grinding off the outer strip, or c) laser welding the edges of the rolled sheet or strip in a vacuum, oxygen reducing atmosphere, or inert atmosphere. In still another and/or additional non-limiting process, the near net frame of the medical device or portion of the medical device (e.g., frame of the medical device, etc.), blank, rod, tube, etc. of the refractory metal alloy is formed by consolidating metal powder. In this process, fine particles of metal (e.g., Re, W, Mo, Ti, Cu, Ni, Cr, etc.) along with any additives are mixed to form a homogenous blend of particles. Typically, the average particle size of the metal powders is less than about 200 mesh (e.g., less than 74 microns; 2-74 microns and all values and ranges therebetween). A larger average particle size can interfere with the proper mixing of the metal powders and/or adversely affect one or more physical properties of the near net frame of the medical device or portion of the medical device (e.g., frame of the medical device, etc.), blank, rod, tube, etc. formed from the metal powders. In one non-limiting embodiment, the average particle size of the metal powders is less than about 230 mesh (e.g., less than 63 microns). In another and/or alternative non-limiting embodiment, the average particle size of the metal powders is about 2-63 microns, and more particularly about 5-40 microns. As can be appreciated, smaller average particle sizes can be used. The purity of the metal powders should be selected so the metal powders contain very low levels of carbon, oxygen, and nitrogen. Typically, the carbon content of the metal powder used to form the refractory metal alloy is less than about 100 ppm, the oxygen content is less than about 50 ppm, and the nitrogen content is less than about 20 ppm. Typically, metal powder used to form the refractory metal alloy has a purity grade of at least 99.9 and more typically at least about 99.95. The blend of metal powder is then pressed together to form a solid solution of the refractory metal alloy into a near net medical device or portion of the medical device (e.g., frame of the medical device, etc.), blank, rod, tube, etc. Typically, the pressing process is by an isostatic process (i.e., uniform pressure applied from all sides on the metal powder); however other processes can be used. When the metal powders are pressed together isostatically, cold isostatic pressing (CIP) is typically used to consolidate the metal powders; however, this is not required. The pressing process can be performed in an inert atmosphere, an oxygen-reducing atmosphere (e.g., hydrogen, argon and hydrogen mixture, etc.) and/or under a vacuum; however, this is not required. The average density of the near net medical device or portion of the medical device (e.g., frame of the medical device, etc.), blank, rod, tube, etc., achieved by pressing together the metal powders is about 80-95% (and all values and ranges therebetween) of the final average density of the near net medical device or portion of the medical device (e.g., frame of the medical device, etc.), blank, rod, tube, etc., or about 70-99% (and all values and ranges therebetween) the minimum theoretical density of the refractory metal alloy. Pressing pressures of at least about 300 MPa (e.g., 300-800 MPa and all values and ranges therebetween) are generally used. Generally, the pressing pressure is about 400-700 MPa; however, other pressures can be used. After the metal powders are pressed together, the pressed metal powders are sintered at a temperature of at least 1600° C. (e.g., 1600-3500° C. and all values and ranges therebetween) to partially or fully fuse the metal powders together to form the near net medical device or portion of the medical device (e.g., frame of the medical device, etc.), blank, rod, tube, etc. The sintering of the consolidated metal powder can be performed in an oxygen-reducing atmosphere (e.g., helium, argon, hydrogen, argon, and hydrogen mixture, etc.) and/or under a vacuum; however, this is not required. At the high sintering temperatures, a high hydrogen atmosphere will reduce both the amount of carbon and oxygen in the formed near net medical device or portion of the medical device (e.g., frame of the medical device, etc.), blank, rod, tube, etc. The sintered metal powder generally has an as-sintered average density of about 90-99.9% (and all values and ranges therebetween) the minimum theoretical density of the refractory metal alloy. Typically, the sintered refractory metal alloy has a final average density of at least about 5 gm/cc (e.g., 5-20 gm/cc and all values and ranges therebetween), and typically at least about 8.3 gm/cc, and can be up to or greater than about 16 gm/cc; however, this is not required. The density of the formed near net medical device or portion of the medical device (e.g., frame of the medical device, etc.), blank, rod, tube, etc., will generally depend on the type of refractory metal alloy used.

In accordance with another and/or alternative non-limiting aspect of the present disclosure, when a solid rod of the refractory metal alloy is formed, the rod is then formed into a tube prior to reducing the outer cross-sectional area or diameter of the rod. The rod can be formed into a tube by a variety of processes such as, but not limited to, cutting or drilling (e.g., gun drilling, etc.) or by cutting (e.g., EDM, EDM sinker, wire EDM, etc.) or by 3D printing. The cavity or passageway formed in the rod typically is formed fully through the rod; however, this is not required.

In yet a further and/or alternative non-limiting aspect of the present disclosure, the near net medical device or portion of the medical device (e.g., frame of the medical device, etc.), blank, rod, tube, etc., can optionally be cleaned and/or polished after the near net medical device or portion of the medical device (e.g., frame of the medical device, etc.), blank, rod, tube, etc., has been form; however, this is not required. Typically, the near net medical device or portion of the medical device (e.g., frame of the medical device, etc.), blank, rod, tube, etc., is cleaned and/or polished prior to being further processed; however, this is not required. When a rod of the refractory metal alloy is formed into a tube, the formed tube is typically cleaned and/or polished prior to being further processed; however, this is not required. When the near net medical device or portion of the medical device (e.g., frame of the medical device, etc.), blank, rod, tube, etc. is resized and/or annealed, the near net medical device or portion of the medical device (e.g., frame of the medical device, etc.), blank, rod, tube, etc., is typically cleaned and/or polished prior to and/or after each or after a series of resizing and/or annealing processes; however, this is not required. The cleaning and/or polishing of the near net medical device or portion of the medical device (e.g., frame of the medical device, etc.), blank, rod, tube, etc., is used to remove impurities and/or contaminants from the surfaces of the near net medical device or portion of the medical device (e.g., frame of the medical device, etc.), blank, rod, tube, etc. Impurities and contaminants can become incorporated into the refractory metal alloy during the processing of the near net medical device or portion of the medical device (e.g., frame of the medical device, etc.), blank, rod, tube, etc. The inadvertent incorporation of impurities and contaminants in the near net medical device or portion of the medical device (e.g., frame of the medical device, etc.), blank, rod, tube, etc., can result in an undesired amount of carbon, nitrogen, and/or oxygen, and/or other impurities in the refractory metal alloy. The inclusion of impurities and contaminants in the refractory metal alloy can result in premature micro-cracking of the refractory metal alloy and/or an adverse effect on one or more physical properties of the refractory metal alloy (e.g., decrease in tensile elongation, increased ductility, increased brittleness, etc.). The cleaning of the refractory metal alloy can be accomplished by a variety of techniques such as, but not limited to, 1) using a solvent (e.g., acetone, methyl alcohol, etc.) and wiping the refractory metal alloy with a Kimwipe or other appropriate towel, 2) by at least partially dipping or immersing the refractory metal alloy in a solvent and then ultrasonically cleaning the refractory metal alloy, and/or 3) by at least partially dipping or immersing the refractory metal alloy in a pickling solution. As can be appreciated, the refractory metal alloy can be cleaned in other or additional ways. If the refractory metal alloy is to be polished, the refractory metal alloy is generally polished by use of a polishing solution that typically includes an acid solution; however, this is not required. In one non-limiting example, the polishing solution includes sulfuric acid; however, other or additional acids can be used. In one non-limiting polishing solution, the polishing solution can include by volume 60-95% sulfuric acid and 5-40% de-ionized water (DI water). Typically, the polishing solution that includes an acid will increase in temperature during the making of the solution and/or during the polishing procedure. As such, the polishing solution is typically stirred and/or cooled during making of the solution and/or during the polishing procedure. The temperature of the polishing solution is typically about 20-100° C. (and all values and ranges therebetween), and typically greater than about 25° C. One non-limiting polishing technique that can be used is an electropolishing technique. When an electropolishing technique is used, a voltage of about 2-30 V (and all values and ranges therebetween), and typically about 5-12 V is applied to the near net medical device or portion of the medical device (e.g., frame of the medical device, etc.), blank, rod, tube, etc. during the polishing process; however, it will be appreciated that other voltages can be used. The time used to polish the refractory metal alloy is dependent on both the size of the near net medical device or portion of the medical device (e.g., frame of the medical device, etc.), blank, rod, tube, etc. and the amount of material that needs to be removed from the near net medical device or portion of the medical device (e.g., frame of the medical device, etc.), blank, rod, tube, etc. The near net medical device or portion of the medical device (e.g., frame of the medical device, etc.), blank, rod, tube, etc. can be processed by use of a two-step polishing process wherein the refractory metal alloy piece is at least partially immersed in the polishing solution for a given period (e.g., 0.1-15 minutes, etc.), rinsed (e.g., DI water, etc.) for a short period of time (e.g., 0.02-1 minute, etc.), and then flipped over and at least partially immersed in the solution again for the same or similar duration as the first time; however, this is not required. The refractory metal alloy can be rinsed (e.g., DI water, etc.) for a period of time (e.g., 0.01-5 minutes, etc.) before rinsing with a solvent (e.g., acetone, methyl alcohol, etc.); however, this is not required. The refractory metal alloy can be dried (e.g., exposure to the atmosphere, maintained in an inert gas environment, etc.) on a clean surface. These polishing procedures can be repeated until the desired amount of polishing of the near net medical device or portion of the medical device (e.g., frame of the medical device, etc.), blank, rod, tube, etc. is achieved. The near net medical device or portion of the medical device (e.g., frame of the medical device, etc.), blank, rod, tube, etc. can be uniformly electropolished or selectively electropolished. When the near net medical device or portion of the medical device (e.g., frame of the medical device, etc.), blank, rod, tube, etc. is selectively electropolished, the selective electropolishing can be used to obtain different surface characteristics of the near net medical device or portion of the medical device (e.g., frame of the medical device, etc.), blank, rod, tube, etc. and/or selectively expose one or more regions of the near net medical device or portion of the medical device (e.g., frame of the medical device, etc.), blank, rod, tube, etc.; however, this is not required.

In still yet a further and/or alternative non-limiting aspect of the present disclosure, the near net medical device or portion of the medical device (e.g., frame of the medical device, etc.), blank, rod, tube, etc., can be resized to the desired dimension of the medical device. In one non-limiting embodiment, the cross-sectional area or diameter of the near net medical device or portion of the medical device (e.g., frame of the medical device, etc.), blank, rod, tube, etc., is reduced to a final near net medical device or portion of the medical device (e.g., frame of the medical device, etc.), blank, rod, tube, etc., dimension in a single step or by a series of steps. The reduction of the outer cross-sectional area or diameter of the near net medical device or portion of the medical device (e.g., frame of the medical device, etc.), blank, rod, tube, etc. may be obtained by centerless grinding, turning, electropolishing, drawing process, grinding, laser cutting, shaving, polishing, EDM cutting, etc. The outer cross-sectional area or diameter size of the near net medical device or portion of the medical device (e.g., frame of the medical device, etc.), blank, rod, tube, etc., can be reduced by the use of one or more drawing processes; however, this is not required. During the drawing process, care should be taken to not form micro-cracks in the near net medical device or portion of the medical device (e.g., frame of the medical device, etc.), blank, rod, tube, etc., during the reduction of the near net medical device or portion of the medical device (e.g., frame of the medical device, etc.), blank, rod, tube, etc., outer cross-sectional area or diameter.

In another and/or alternative non-limiting aspect of the present disclosure, the near net medical device or portion of the medical device (e.g., frame of the medical device, etc.), blank, rod, tube, etc. general if not reduced in cross-sectional area by more about 25% (e.g., 0.1-25% and all values and ranges therebetween) each time the near net medical device or portion of the medical device (e.g., frame of the medical device, etc.), blank, rod, tube, etc. is drawn down in size. When the near net medical device or portion of the medical device (e.g., frame of the medical device, etc.), blank, rod, tube, etc. optionally includes a nitride layer, the nitrided layer can optionally function as a lubricating surface during the drawing process to facilitate in the drawing of the near net medical device or portion of the medical device (e.g., frame of the medical device, etc.), blank, rod, tube, etc. Generally, the near net medical device or portion of the medical device (e.g., frame of the medical device, etc.), blank, rod, tube, etc. is reduced in cross-sectional area by about 0.1-20% each time the near net medical device or portion of the medical device (e.g., frame of the medical device, etc.), blank, rod, tube, etc. is drawn through a reducing mechanism. In another and/or alternative non-limiting process step, the near net medical device or portion of the medical device (e.g., frame of the medical device, etc.), blank, rod, tube, etc. is reduced in cross-sectional area by about 1-15% each time the near net medical device or portion of the medical device (e.g., frame of the medical device, etc.), blank, rod, tube, etc. is drawn through a reducing mechanism. In still another and/or alternative non-limiting process step, the near net medical device or portion of the medical device (e.g., frame of the medical device, etc.), blank, rod, tube, etc. is reduced in cross-sectional area by about 2-15% each time the near net medical device or portion of the medical device (e.g., frame of the medical device, etc.), blank, rod, tube, etc. is drawn through reducing mechanism. In yet another one non-limiting process step, the near net medical device or portion of the medical device (e.g., frame of the medical device, etc.), blank, rod, tube, etc. is reduced in cross-sectional area by about 5-10% each time the near net medical device or portion of the medical device (e.g., frame of the medical device, etc.), blank, rod, tube, etc. is drawn through reducing mechanism. In another and/or alternative non-limiting embodiment of the disclosure, the near net medical device or portion of the medical device (e.g., frame of the medical device, etc.), blank, rod, tube, etc. of refractory metal alloy is drawn through a die to reduce the cross-sectional area of the near net medical device or portion of the medical device (e.g., frame of the medical device, etc.), blank, rod, tube, etc. Generally, before drawing the near net medical device or portion of the medical device (e.g., frame of the medical device, etc.), blank, rod, tube, etc. through a die, one end of the near net medical device or portion of the medical device (e.g., frame of the medical device, etc.), blank, rod, tube, etc. is narrowed down (nosed) so as to allow it to be fed through the die; however, this is not required. The tube drawing process is typically a cold drawing process or a plug drawing process through a die. When a cold drawing or mandrel drawing process is used, a lubricant (e.g., molybdenum paste, grease, etc.) is typically coated on the outer surface of the near net medical device or portion of the medical device (e.g., frame of the medical device, etc.), blank, rod, tube, etc. and the near net medical device or portion of the medical device (e.g., frame of the medical device, etc.), blank, rod, tube, etc. is then drawn though the die. Typically, little or no heat is used during the cold drawing process. After the near net medical device or portion of the medical device (e.g., frame of the medical device, etc.), blank, rod, tube, etc. has been drawn through the die, the outer surface of the near net medical device or portion of the medical device (e.g., frame of the medical device, etc.), blank, rod, tube, etc. is typically cleaned with a solvent to remove the lubricant so as to limit the amount of impurities that are incorporated in the refractory metal alloy; however, this is not required. This cold drawing process can be repeated several times until the desired outer cross-sectional area or diameter, inner cross-sectional area or diameter and/or wall thickness of the near net medical device or portion of the medical device (e.g., frame of the medical device, etc.), blank, rod, tube, etc. is achieved. A plug drawing process can also or alternatively be used to size the near net medical device or portion of the medical device (e.g., frame of the medical device, etc.), blank, rod, tube, etc. The plug drawing process typically does not use a lubricant during the drawing process. The plug drawing process typically includes a heating step to heat the near net medical device or portion of the medical device (e.g., frame of the medical device, etc.), blank, rod, tube, etc. prior and/or during the drawing of the near net medical device or portion of the medical device (e.g., frame of the medical device, etc.), blank, rod, tube, etc. through the die. The elimination of the use of a lubricant can reduce the incidence of impurities being introduced into the refractory metal alloy during the drawing process. During the plug drawing process, the near net medical device or portion of the medical device (e.g., frame of the medical device, etc.), blank, rod, tube, etc. can be protected from oxygen by use of a vacuum environment, a non-oxygen environment (e.g., hydrogen, argon and hydrogen mixture, nitrogen, nitrogen and hydrogen, etc.) or an inert environment. One non-limiting protective environment includes argon, hydrogen or argon and hydrogen; however, other or additional inert gasses can be used. As indicated above, the near net medical device or portion of the medical device (e.g., frame of the medical device, etc.), blank, rod, tube, etc. is typically cleaned after each drawing process to remove impurities and/or other undesired materials from the surface of the near net medical device or portion of the medical device (e.g., frame of the medical device, etc.), blank, rod, tube, etc.; however, this is not required. Typically, the near net medical device or portion of the medical device (e.g., frame of the medical device, etc.), blank, rod, tube, etc. should be shielded from oxygen and nitrogen when the temperature of the near net medical device or portion of the medical device (e.g., frame of the medical device, etc.), blank, rod, tube, etc. is increased to above 500° C., and typically above 450° C., and more typically above 400° C.; however, this is not required. When the near net medical device or portion of the medical device (e.g., frame of the medical device, etc.), blank, rod, tube, etc. is heated to temperatures above about 400-500° C., the near net medical device or portion of the medical device (e.g., frame of the medical device, etc.), blank, rod, tube, etc. tends to begin forming nitrides and/or in the presence of nitrogen and oxygen. In these higher temperature environments, a hydrogen environment, an argon and hydrogen environment, etc. is generally used. When the near net medical device or portion of the medical device (e.g., frame of the medical device, etc.), blank, rod, tube, etc. is drawn at temperatures below 400-500° C., the near net medical device or portion of the medical device (e.g., frame of the medical device, etc.), blank, rod, tube, etc. can be exposed to air with little or no adverse effects; however, an inert or slightly reducing environment is generally more desirable.

In another and/or alternative non-limiting aspect of the present disclosure, the near net medical device or portion of the medical device (e.g., frame of the medical device, etc.), blank, rod, tube, etc. is cooled after being annealed; however, this is not required. Generally, the near net medical device or portion of the medical device (e.g., frame of the medical device, etc.), blank, rod, tube, etc. is cooled at a fairly quick rate after being annealed so as to inhibit or prevent the formation of a sigma phase in the refractory metal alloy; however, this is not required. Generally, the near net medical device or portion of the medical device (e.g., frame of the medical device, etc.), blank, rod, tube, etc. is cooled at a rate of at least about 50° C. per minute (e.g., 50-500° C. per minute and all values and ranges therebetween) after being annealed, typically at least about 75° C. per minute after being annealed, more typically at least about 100° C. per minute after being annealed, even more typically about 100-400° C. per minute after being annealed, still even more typically about 150-350° C. per minute after being annealed, and yet still more typically about 200-300° C. per minute after being annealed, and still yet even more typically about 250-280° C. per minute after being annealed; however, this is not required.

In another and/or alternative non-limiting aspect of the present disclosure, the near net medical device or portion of the medical device (e.g., frame of the medical device, etc.), blank, rod, tube, etc. is annealed after one or more drawing processes. The refractory metal alloy blank, rod, tube, etc. can be annealed after each drawing process or after a plurality of drawing processes. The refractory metal alloy blank, rod, tube, etc. is typically annealed prior to about a 60% cross-sectional area size reduction of the refractory metal alloy blank, rod, tube, etc. In other words, the near net medical device or portion of the medical device (e.g., frame of the medical device, etc.), blank, rod, tube, etc. should not be reduced in cross-sectional area by more than 60% before being annealed (e.g., 0.1-60% reduction and all values and ranges therebetween). A too-large reduction in the cross-sectional area of the refractory metal alloy blank, rod, tube, etc. during the drawing process prior to the near net medical device or portion of the medical device (e.g., frame of the medical device, etc.), blank, rod, tube, etc. being annealed can result in micro-cracking of the near net medical device or portion of the medical device (e.g., frame of the medical device, etc.), blank, rod, tube, etc. In one non-limiting processing step, the refractory metal alloy blank, rod, tube, etc. is annealed prior to about a 50% cross-sectional area size reduction of the refractory metal alloy blank, rod, tube, etc. In another and/or alternative non-limiting processing step, the refractory metal alloy blank, rod, tube, etc. is annealed prior to about a 45% cross-sectional area size reduction of the refractory metal alloy blank, rod, tube, etc. In accordance with another and/or alternative non-limiting aspect of the present disclosure, the refractory metal alloy blank, rod, tube, etc. is annealed prior to about a 1-45% cross-sectional area size reduction of the refractory metal alloy blank, rod, tube, etc. In yet another and/or alternative non-limiting processing step, the refractory metal alloy blank, rod, tube, etc. is annealed prior to about a 5-30% cross-sectional area size reduction of the refractory metal alloy blank, rod, tube, etc. In still yet another and/or alternative non-limiting processing step, the refractory metal alloy blank, rod, tube, etc. is annealed prior to about a 5-15% cross-sectional area size reduction of the refractory metal alloy blank, rod, tube, etc.

In accordance with another and/or alternative non-limiting aspect of the present disclosure, when the near net medical device or portion of the medical device (e.g., frame of the medical device, etc.), blank, rod, tube, etc. is annealed, the near net medical device or portion of the medical device (e.g., frame of the medical device, etc.), blank, rod, tube, etc. is typically heated to a temperature of about 500-1700° C. (and all values and ranges therebetween) for a period of about 1-200 minutes (and all values and ranges therebetween); however, other temperatures and/or times can be used. In one non-limiting processing step, the near net medical device or portion of the medical device (e.g., frame of the medical device, etc.), blank, rod, tube, etc. is annealed at a temperature of about 1000-1600° C. for about 2-100 minutes. In another non-limiting processing step, the near net medical device or portion of the medical device (e.g., frame of the medical device, etc.), blank, rod, tube, etc. is annealed at a temperature of about 1100-1500° C. for about 5-30 minutes. The annealing process typically occurs in an inert environment or an oxygen-reducing environment so as to limit the amount of impurities that may embed themselves in the refractory metal alloy during the annealing process. One non-limiting oxygen-reducing environment that can be used during the annealing process is a hydrogen environment; however, it can be appreciated that a vacuum environment can be used or one or more other or additional gasses can be used to create the oxygen-reducing environment. At the annealing temperatures, a hydrogen-containing atmosphere can further reduce the amount of oxygen in the near net medical device or portion of the medical device (e.g., frame of the medical device, etc.), blank, rod, tube, etc. The chamber in which the near net medical device or portion of the medical device (e.g., frame of the medical device, etc.), blank, rod, tube, etc. is annealed should be substantially free (e.g., 0-50 ppm and all value and ranges therebetween) of impurities (e.g., carbon, oxygen, nitrogen, etc.) so as to limit the amount of impurities that can embed themselves in the near net medical device or portion of the medical device (e.g., frame of the medical device, etc.), blank, rod, tube, etc. during the annealing process. The annealing chamber typically is formed of a material that will not impart impurities to the near net medical device or portion of the medical device (e.g., frame of the medical device, etc.), blank, rod, tube, etc. as the near net medical device or portion of the medical device (e.g., frame of the medical device, etc.), blank, rod, tube, etc. is being annealed. A non-limiting material that can be used to form the annealing chamber includes, but is not limited to, molybdenum, rhenium, tungsten, molybdenum TZM alloy, cobalt, chromium, ceramic, etc. When the near net medical device or portion of the medical device (e.g., frame of the medical device, etc.), blank, rod, tube, etc. is restrained in the annealing chamber, the restraining apparatuses that are used to contact the near net medical device or portion of the medical device (e.g., frame of the medical device, etc.), blank, rod, tube, etc. are typically formed of materials that will not introduce impurities to the refractory metal alloy during the processing of the near net medical device or portion of the medical device (e.g., frame of the medical device, etc.), blank, rod, tube, etc. Non-limiting examples of materials that can be used to at least partially form the restraining apparatuses include, but are not limited to, molybdenum, titanium, yttrium, zirconium, rhenium, cobalt, chromium, tantalum, and/or tungsten. In one non-limiting embodiment, when the refractory metal alloy is exposed to temperatures above 150° C. for any process step including annealing, the materials that contact the refractory metal alloy during the processing of the refractory metal alloy are typically made from chromium, cobalt, molybdenum, rhenium, tantalum and/or tungsten. When the refractory metal alloy is processed at lower temperatures (i.e., 150° C. or less), materials made from Teflon™ parts can also or alternatively be used.

In accordance with another and/or alternative non-limiting aspect of the present disclosure, the parameters for annealing can be changed as the near net medical device or portion of the medical device (e.g., frame of the medical device, etc.), blank, rod, tube, etc. as the cross-sectional area or diameter; and/or wall thickness of the near net medical device or portion of the medical device (e.g., frame of the medical device, etc.), blank, rod, tube, etc. are changed. It has been found that good grain size characteristics of the near net medical device or portion of the medical device (e.g., frame of the medical device, etc.), blank, rod, tube, etc. can be achieved when the annealing parameters are varied as the parameters of the near net medical device or portion of the medical device (e.g., frame of the medical device, etc.), blank, rod, tube, etc. change. For example, as the wall thickness is reduced, the annealing temperature is correspondingly reduced; however, the times for annealing can be increased. As can be appreciated, the annealing temperatures of the near net medical device or portion of the medical device (e.g., frame of the medical device, etc.), blank, rod, tube, etc. can be decreased as the wall thickness decreases, but the annealing times can remain the same or also be reduced as the wall thickness reduces. After each annealing process, the grain size of the metal in the near net medical device or portion of the medical device (e.g., frame of the medical device, etc.), blank, rod, tube, etc. should be no greater than 4 ASTM. Generally, the grain size range is about 4-20 ASTM (and all values and ranges therebetween). It is believed that as the annealing temperature is reduced as the wall thickness reduces, small grain sizes can be obtained. The grain size of the metal in the near net medical device or portion of the medical device (e.g., frame of the medical device, etc.), blank, rod, tube, etc. should be as uniform as possible. In addition, the sigma phase of the metal in the near net medical device or portion of the medical device (e.g., frame of the medical device, etc.), blank, rod, tube, etc. should be as reduced as much as possible. The sigma phase is a spherical, elliptical or tetragonal crystalline shape in the refractory metal alloy. After the final drawing of the near net medical device or portion of the medical device (e.g., frame of the medical device, etc.), blank, rod, tube, etc., a final annealing of the near net medical device or portion of the medical device (e.g., frame of the medical device, etc.), blank, rod, tube, etc. can be done for final strengthening of the near net medical device or portion of the medical device (e.g., frame of the medical device, etc.), blank, rod, tube, etc.; however, this is not required. This final annealing process, when used, generally occurs at a temperature of about 500-1600° C. (and all values and ranges therebetween) for at least about 1 minute; however, other temperatures and/or time periods can be used.

In accordance with another and/or alternative non-limiting aspect of the present disclosure, the near net medical device or portion of the medical device (e.g., frame of the medical device, etc.), blank, rod, tube, etc. can be cleaned prior to and/or after being annealed. The cleaning process is designed to remove impurities, lubricants (e.g., nitride compounds, molybdenum paste, grease, oxides, carbides, etc.) and/or other materials from the surfaces of the near net medical device or portion of the medical device (e.g., frame of the medical device, etc.), blank, rod, tube, etc. Impurities that are on one or more surfaces of the near net medical device or portion of the medical device (e.g., frame of the medical device, etc.), blank, rod, tube, etc. can become permanently embedded into the near net medical device or portion of the medical device (e.g., frame of the medical device, etc.), blank, rod, tube, etc. during the annealing processes. These imbedded impurities can adversely affect the physical properties of the refractory metal alloy as the near net medical device or portion of the medical device (e.g., frame of the medical device, etc.), blank, rod, tube, etc. is formed into a medical device, and/or can adversely affect the operation and/or life of the medical device. In one non-limiting embodiment of the disclosure, the cleaning process includes a delubrication or degreasing process which is typically followed by pickling process; however, this is not required. The delubrication or degreasing process followed by pickling process is typically used when a lubricant has been used on the near net medical device or portion of the medical device (e.g., frame of the medical device, etc.), blank, rod, tube, etc. during a drawing process. Lubricants commonly include carbon compounds, nitride compounds, molybdenum paste, and other types of compounds that can adversely affect the refractory metal alloy if such compounds and/or elements in such compounds become associated and/or embedded with the refractory metal alloy during an annealing process. The delubrication or degreasing process can be accomplished by a variety of techniques such as, but not limited to, 1) using a solvent (e.g., acetone, methyl alcohol, etc.) and wiping the refractory metal alloy with a Kimwipe or other appropriate towel, 2) by at least partially dipping or immersing the refractory metal alloy in a solvent and then ultrasonically cleaning the refractory metal alloy, 3) sand blasting the refractory metal alloy, and/or 4) chemical etching the refractory metal alloy. As can be appreciated, the refractory metal alloy can be delubricated or degreased in other or additional ways. After the near net medical device or portion of the medical device (e.g., frame of the medical device, etc.), blank, rod, tube, etc. has been delubricated or degreased, the near net medical device or portion of the medical device (e.g., frame of the medical device, etc.), blank, rod, tube, etc. can be further cleaned by use of a pickling process; however, this is not required. The pickling process (when used) includes the use of one or more acids to remove impurities from the surface of the near net medical device or portion of the medical device (e.g., frame of the medical device, etc.), blank, rod, tube, etc. Non-limiting examples of acids that can be used as the pickling solution include, but are not limited to, nitric acid, acetic acid, sulfuric acid, hydrochloric acid, and/or hydrofluoric acid. These acids are typically analytical reagent (ACS) grade acids. The acid solution and acid concentration are selected to remove oxides and other impurities on the near net medical device or portion of the medical device (e.g., frame of the medical device, etc.), blank, rod, tube, etc. surface without damaging or over-etching the surface of the near net medical device or portion of the medical device (e.g., frame of the medical device, etc.), blank, rod, tube, etc. A near net medical device or portion of the medical device (e.g., frame of the medical device, etc.), blank, rod, tube, etc. surface that includes a large amount of oxides and/or nitrides typically requires a stronger pickling solution and/or long pickling process times. Non-limiting examples of pickling solutions include 1) 25-60% DI water (and all values and ranges therebetween), 30-60% nitric acid (and all values and ranges therebetween), and 2-20% sulfuric acid (and all values and ranges therebetween); 2) 40-75% acetic acid (and all values and ranges therebetween), 10-35% nitric acid (and all values and ranges therebetween), and 1-12% hydrofluoric acid (and all values and ranges therebetween); and 3) 50-100% hydrochloric acid (and all values and ranges therebetween). As can be appreciated, one or more different pickling solutions can be used during the pickling process. During the pickling process, the near net medical device or portion of the medical device (e.g., frame of the medical device, etc.), blank, rod, tube, etc. is fully or partially immersed in the pickling solution for a sufficient amount of time to remove the impurities from the surface of the near net medical device or portion of the medical device (e.g., frame of the medical device, etc.), blank, rod, tube, etc. Typically, the time period for pickling is about 2-120 seconds (and all values and ranges therebetween); however, other time periods can be used. After the near net medical device or portion of the medical device (e.g., frame of the medical device, etc.), blank, rod, tube, etc. has been pickled, the near net medical device or portion of the medical device (e.g., frame of the medical device, etc.), blank, rod, tube, etc. is typically rinsed with a water (e.g., DI water, etc.) and/or a solvent (e.g., acetone, methyl alcohol, etc.) to remove any pickling solution from the near net medical device or portion of the medical device (e.g., frame of the medical device, etc.), blank, rod, tube, etc. and then the near net medical device or portion of the medical device (e.g., frame of the medical device, etc.), blank, rod, tube, etc. is allowed to dry. The near net medical device or portion of the medical device (e.g., frame of the medical device, etc.), blank, rod, tube, etc. may be keep in a protective environment during the rinse and/or drying process to inhibit or prevent oxides from reforming on the surface of the near net medical device or portion of the medical device (e.g., frame of the medical device, etc.), blank, rod, tube, etc. prior to the near net medical device or portion of the medical device (e.g., frame of the medical device, etc.), blank, rod, tube, etc. being drawn and/or annealed; however, this is not required.

In accordance with another and/or alternative non-limiting aspect of the present disclosure, the near net medical device or portion of the medical device (e.g., frame of the medical device, etc.), blank, rod, tube, etc., after a) being formed to the desired green shape, b) after being formed to have the desired outer cross-sectional area or diameter, and/or c) after being formed to have the desired inner cross-sectional area or diameter and/or wall thickness, can then be cut and/or etched to at least partially form the desired configuration of the medical device or portion of the medical device (e.g., frame of the medical device, etc.) (e.g., stent, TAV valve, etc.). The near net medical device or portion of the medical device (e.g., frame of the medical device, etc.), blank, rod, tube, etc. can be cut or otherwise formed by one or more processes (e.g., centerless grinding, turning, electropolishing, drawing process, grinding, laser cutting, shaving, polishing, EDM cutting, etching, micro-machining, laser micro-machining, micro-molding, machining, etc.). As can be appreciated, a portion or all of the medical device or portion of the medical device (e.g., frame of the medical device, etc.) can be formed by 3D printing. In one non limiting embodiment of the disclosure, the refractory metal alloy used to partially or fully form the near net medical device or portion of the medical device (e.g., frame of the medical device, etc.), blank, rod, tube, etc. is at least partially cut by a laser. The laser is typically desired to have a beam strength which can heat the refractory metal alloy near net medical device or portion of the medical device (e.g., frame of the medical device, etc.), blank, rod, tube, etc. to a temperature up to at least about 2200-2300° C. In one non-limiting aspect of this embodiment, a pulsed Nd:YAG neodymium-doped yttrium aluminum garnet (Nd:Y₃Al₅O₁₂) or CO₂ laser is used to at least partially cut a pattern of a medical device or portion of the medical device (e.g., frame of the medical device, etc.) out of the refractory metal alloy blank, rod, tube, etc. In accordance with another and/or alternative non-limiting aspect of this embodiment, the cutting of the refractory metal alloy that is used to partially or fully form the near net medical device or portion of the medical device (e.g., frame of the medical device, etc.), blank, rod, tube, etc. by the laser can occur in a vacuum environment, an oxygen-reducing environment, or an inert environment; however, this is not required. It has been found that laser cutting of the near net medical device or portion of the medical device (e.g., frame of the medical device, etc.), blank, rod, tube, etc. in a non-protected environment can result in impurities being introduced into the cut near net medical device or portion of the medical device (e.g., frame of the medical device, etc.), blank, rod, tube, etc., which introduced impurities can induce micro-cracking of the near net medical device or portion of the medical device (e.g., frame of the medical device, etc.), blank, rod, tube, etc. during the cutting of the near net medical device or portion of the medical device (e.g., frame of the medical device, etc.), blank, rod, tube, etc. One non-limiting oxygen-reducing environment includes a combination of argon and hydrogen; however, a vacuum environment, an inert environment, or other or additional gasses can be used to form the oxygen reducing environment. In still another and/or alternative non-limiting aspect of this embodiment, the refractory metal alloy that used to partially or fully form the near net medical device or portion of the medical device (e.g., frame of the medical device, etc.), blank, rod, tube, etc. is stabilized so as to limit or prevent vibration of the near net medical device or portion of the medical device (e.g., frame of the medical device, etc.), blank, rod, tube, etc. during the cutting process. The apparatus used to stabilize the near net medical device or portion of the medical device (e.g., frame of the medical device, etc.), blank, rod, tube, etc. can be formed of molybdenum, rhenium, tungsten, tantalum, cobalt, chromium, molybdenum TZM alloy, ceramic, etc. so as to not introduce contaminants to the near net medical device or portion of the medical device (e.g., frame of the medical device, etc.), blank, rod, tube, etc. during the cutting process; however, this is not required. Vibrations in the near net medical device or portion of the medical device (e.g., frame of the medical device, etc.), blank, rod, tube, etc. during the cutting of the near net medical device or portion of the medical device (e.g., frame of the medical device, etc.), blank, rod, tube, etc. can result in the formation of micro-cracks in the near net medical device or portion of the medical device (e.g., frame of the medical device, etc.), blank, rod, tube, etc. as the near net medical device or portion of the medical device (e.g., frame of the medical device, etc.), blank, rod, tube, etc. is cut. The average amplitude of vibration during the cutting of the near net medical device or portion of the medical device (e.g., frame of the medical device, etc.), blank, rod, tube, etc. is generally no more than about 150% (0-150% and all values and ranges therebetween) of the wall thickness of the near net medical device or portion of the medical device (e.g., frame of the medical device, etc.), blank, rod, tube, etc.; however, this is not required. In one non-limiting aspect of this embodiment, the average amplitude of vibration is no more than about 100% of the wall thickness of the near net medical device or portion of the medical device (e.g., frame of the medical device, etc.), blank, rod, tube, etc. In another non-limiting aspect of this embodiment, the average amplitude of vibration is no more than about 75% of the wall thickness of the near net medical device or portion of the medical device (e.g., frame of the medical device, etc.), blank, rod, tube, etc. In still another non-limiting aspect of this embodiment, the average amplitude of vibration is no more than about 50% of the wall thickness of the near net medical device or portion of the medical device (e.g., frame of the medical device, etc.), blank, rod, tube, etc. In yet another non-limiting aspect of this embodiment, the average amplitude of vibration is no more than about 25% of the wall thickness of the near net medical device or portion of the medical device (e.g., frame of the medical device, etc.), blank, rod, tube, etc. In still yet another non-limiting aspect of this embodiment, the average amplitude of vibration is no more than about 15% of the wall thickness of the near net medical device, blank, rod, tube, etc.

In accordance with another and/or alternative non-limiting aspect of the present disclosure, the refractory metal alloy that is used to partially or fully form the near net medical device or portion of the medical device (e.g., frame of the medical device, etc.), after being formed into its final or near final shape, can optionally be cleaned, polished, sterilized, nitrided, etc. In one non-limiting embodiment of the disclosure, the medical device or portion of the medical device (e.g., frame of the medical device, etc.) is electropolished. In one non-limiting aspect of this embodiment, the medical device or portion of the medical device (e.g., frame of the medical device, etc.) is cleaned prior to being exposed to the polishing solution; however, this is not required. The cleaning process (when used) can be accomplished by a variety of techniques such as, but not limited to, 1) using a solvent and wiping the medical device or portion of the medical device (e.g., frame of the medical device, etc.) with a Kimwipe or other appropriate towel, and/or 2) by at least partially dipping or immersing the medical device or portion of the medical device (e.g., frame of the medical device, etc.) in a solvent and then ultrasonically cleaning the metal part or medical device or portion of the medical device (e.g., frame of the medical device, etc.). As can be appreciated, the medical device or portion of the medical device (e.g., frame of the medical device, etc.) can be cleaned in other or additional ways. In accordance with another and/or alternative non-limiting aspect of this embodiment, the polishing solution can include one or more acids. In yet another and/or alternative non-limiting aspect of this embodiment, the medical device or portion of the medical device (e.g., frame of the medical device, etc.) is rinsed with water and/or a solvent and allowed to dry to remove polishing solution on the metal part or medical device or portion of the medical device (e.g., frame of the medical device, etc.). In another and/or alternative non-limiting embodiment of the disclosure, the formed medical device or portion of the medical device (e.g., frame of the medical device, etc.) is optionally nitrided. After the medical device or portion of the medical device (e.g., frame of the medical device, etc.) is nitrided, the medical device or portion of the medical device (e.g., frame of the medical device, etc.) is typically cleaned; however, this is not required. During the nitride process, the surface of the medical device or portion of the medical device (e.g., frame of the medical device, etc.) is modified by the present of nitrogen. The nitriding process for the medical device or portion of the medical device (e.g., frame of the medical device, etc.) can be used to increase surface hardness and/or wear resistance of the medical device or portion of the medical device (e.g., frame of the medical device, etc.) and/or limit or present discoloration of the surface of the frame of the medical device or portion of the medical device (e.g., frame of the medical device, etc.). For example, the nitriding process can be used to increase the wear resistance of articulation surfaces or surface wear on the medical device or portion of the medical device (e.g., frame of the medical device, etc.) to extend the life of the medical device or portion of the medical device (e.g., frame of the medical device, etc.), and/or to increase the wear life of mating surfaces on the medical device or portion of the medical device (e.g., frame of the medical device, etc.), and/or to reduce particulate generation from use of the medical device.

In accordance with another and/or alternative non-limiting aspect of the present disclosure, there is provided a prosthetic heart valve that is configured to be inserted into a desired location in the body (e.g., the aortic valve, tricuspid valve, pulmonary valve, mitral valve). The frame of the prosthetic heart valve can be at least partially formed of a plastically-expandable material that permits crimping of the frame to a smaller profile for delivery and expansion of the prosthetic heart valve to a larger profile. The expansion of the crimped frame can be optionally be use of an expansion device such as, but not limited to, a balloon of on a balloon catheter. As can be appreciated, the medical device can be a device other than a prosthetic heart valve (e.g., stent, etc.) that includes a frame that is at least partially formed of a plastically expandable material that permits crimping of the frame to a smaller profile for delivery and expansion of the medical device to a larger profile.

In accordance with another and/or alternative non-limiting aspect of the present disclosure, the use of the refractory metal alloy to partially or fully form the medical device or portion of the medical device (e.g., frame of the medical device, etc.) can be used to increase the strength and/or hardness and/or durability of the medical device or portion of the medical device (e.g., frame of the medical device, etc.) as compared with stainless steel or chromium-cobalt alloys or titanium alloys; thus, less quantity of refractory metal alloy can be used in the medical device or portion of the medical device (e.g., frame of the medical device, etc.) to achieve similar strengths as compared to frames of medical devices formed of different metals. As such, the resulting medical device can be made smaller and less bulky by use of the refractory metal alloy without sacrificing the strength and durability of the medical device. Such a medical device can have a smaller profile, thus can be inserted in smaller areas, openings and/or passageways. The refractory metal alloy also can increase the radial strength of the medical device or portion of the medical device (e.g., frame of the medical device, etc.). For instance, the thickness of the walls of the medical device or portion of the medical device (e.g., frame of the medical device, etc.) and/or the wires used to at least partially form the medical device or portion of the medical device (e.g., frame of the medical device, etc.) can be made thinner and achieve a similar or improved radial strength as compared with thicker walled frames of medical devices formed of stainless steel, titanium alloys or cobalt and chromium alloys. The refractory metal alloy also can improve stress-strain properties, bendability and flexibility of the medical device or portion of the medical device (e.g., frame of the medical device, etc.), thus increase the life of the medical device. For instance, the medical device can be used in regions that subject the medical device to bending. Due to the improved physical properties of the medical device from the refractory metal alloy, the medical device has improved resistance to fracturing in such frequent bending environments. In addition or alternatively, the improved bendability and flexibility of the medical device or portion of the medical device (e.g., frame of the medical device, etc.) due to the use of the refractory metal alloy can enable the medical device to be more easily inserted into various regions of a body. The refractory metal alloy can also reduce the degree of recoil during the crimping and/or expansion of the medical device or portion of the medical device (e.g., frame of the medical device, etc.). For example, the medical device better maintains its crimped form and/or better maintains its expanded form after expansion due to the use of the refractory metal alloy. As such, when the medical device is to be mounted onto a delivery device when the medical device is crimped, the medical device better maintains its smaller profile during the insertion of the medical device into various regions of a body. Also, the medical device better maintains its expanded profile after expansion so as to facilitate in the success of the medical device in the treatment area. In addition to the improved physical properties of the medical device by use of the refractory metal alloy, the refractory metal alloy has improved radiopaque properties as compared to standard materials such as stainless steel or cobalt-chromium alloy, thus reducing or eliminating the need for using marker materials on the medical device. For instance, the refractory metal alloy is believed to at least about 10-20% more radiopaque than stainless steel or cobalt-chromium alloy.

In accordance with another and/or alternative non-limiting aspect of the present disclosure, the use of the refractory metal alloy to form all or a portion of the medical device can result in several advantages over medical devices formed from other materials. These advantages include, but are not limited to:

The refractory metal alloy has increased strength and/or hardness as compared with stainless steel, chromium-cobalt alloys, or titanium alloys, thus a less quantity of refractory metal alloy can be used in the medical device to achieve similar strengths as compared to medical devices formed of different metals. As such, the resulting medical device can be made smaller and less bulky by use of the refractory metal alloy without sacrificing the strength and durability of the medical device. The medical device can also have a smaller profile, thus can be inserted into smaller areas, openings, and/or passageways. The thinner struts of refractory metal alloy to form the frame or other portions of the medical device can be used to form a frame or other portion of the medical device having a strength that would require thicker struts or other structures of the medical device when formed by stainless steel, chromium-cobalt alloys, or titanium alloys.

The increased strength of the refractory metal alloy also results in the increased radial strength of the medical device. For instance, the thickness of the walls of the medical device can be made thinner and achieve a similar or improved radial strength as compared with thicker walled medical devices formed of stainless steel, cobalt and chromium alloy, or titanium alloy.

The refractory metal alloy has improved stress-strain properties, bendability properties, elongation properties, and/or flexibility properties of the medical device compared with stainless steel and chromium-cobalt alloys, thus resulting in an increase life for the medical device. For example, the medical device can be used in regions that subject the medical device to repeated bending. Due to the improved physical properties of the medical device from the refractory metal alloy, the medical device has improved resistance to fracturing in such frequent bending environments. These improved physical properties at least in part result from the composition of the refractory metal alloy, the grain size of the refractory metal alloy, the carbon, oxygen, and nitrogen content of the refractory metal alloy, and/or the carbon/oxygen ratio of the refractory metal alloy.

The refractory metal alloy has a reduced degree of recoil during the crimping and/or expansion of the medical device compared with stainless steel, chromium-cobalt alloys, or titanium alloys. The medical device formed of the refractory metal alloy better maintains its crimped form and/or better maintains its expanded form after expansion due to the use of the refractory metal alloy. As such, when the medical device is to be mounted onto a delivery device when the medical device is crimped, the medical device better maintains its smaller profile during the insertion of the medical device in a body passageway. Also, the medical device better maintains its expanded profile after expansion to facilitate in the success of the medical device in the treatment area.

The use of the refractory metal alloy in the medical device results in the medical device better conforming to an irregularly shaped body passageway when expanded in the body passageway compared to a medical device formed by stainless steel, chromium-cobalt alloys, or titanium alloys.

The refractory metal alloy has improved radiopaque properties compared to standard materials such as stainless steel or cobalt-chromium alloy, thus reducing or eliminating the need for using marker materials on the medical device. For example, the refractory metal alloy is at least about 10-20% more radiopaque than stainless steel or cobalt-chromium alloy.

The refractory metal alloy has improved fatigue ductility when subjected to cold-working compared to the cold-working of stainless steel, chromium-cobalt alloys, or titanium alloys.

The refractory metal alloy has improved durability compared to stainless steel, chromium-cobalt alloys, or titanium alloys.

The refractory metal alloy has improved hydrophilicity compared to stainless steel, chromium-cobalt alloys, or titanium alloys.

The refractory metal alloy has reduced ion release in the body passageway compared to stainless steel, chromium-cobalt alloys, or titanium alloys.

The refractory metal alloy is less of an irritant to the body than stainless steel, cobalt-chromium alloy, or titanium alloys, thus can result in reduced inflammation, faster healing, increased success rates of the medical device. When the medical device is expanded in a body passageway, some minor damage to the interior of the passageway can occur. When the body begins to heal such minor damage, the body has less adverse reaction to the presence of the refractory metal alloy compared to other metals such as stainless steel, cobalt-chromium alloy, or titanium alloy.

The refractory metal alloy has a magnetic susceptibility that is lower that CoCr alloy, TiAlV alloys, and/or stainless steel, thus resulting in less incidence of potential defects to the medical device or complications to the patent after implantation of the medical device when the patient is subjected to an MRI or other medical device that generates a strong magnetic field.

One non-limiting object of the present disclosure is the provision of the refractory metal alloy in accordance with the present disclosure that can be used to partially or fully form a medical device.

Another and/or alternative non-limiting object of the present disclosure is the provision of a medical device that is partially or fully formed of the refractory metal alloy of the present disclosure and which medical device has improved procedural success rates.

Another and/or alternative non-limiting object of the present disclosure is the provision of a method and process for forming the refractory metal alloy in accordance with the present disclosure that inhibits or prevents the formation of micro-cracks during the processing of the refractory metal alloy.

Another and/or alternative non-limiting object of the present disclosure is the provision of a medical device that is partially or fully formed of the refractory metal alloy in accordance with the present disclosure and wherein the medical device has improved physical properties.

Another and/or alternative non-limiting object of the present disclosure is the provision of a medical device that is at least partially formed of the refractory metal alloy in accordance with the present disclosure that has increased strength and/or hardness.

Another and/or alternative non-limiting object of the present disclosure is the provision of a medical device that at least partially includes the refractory metal alloy in accordance with the present disclosure and which refractory metal alloy enables the medical device to be formed with less material without sacrificing the strength of the medical device compared to prior medical devices.

Another and/or alternative non-limiting object of the present disclosure is the provision of a method and process for forming the refractory metal alloy in accordance with the present disclosure to inhibit or prevent the formation of micro-cracks during the processing of the refractory metal alloy into a medical device.

Another and/or alternative non-limiting object of the present disclosure is the provision of a method and process for forming the refractory metal alloy in accordance with the present disclosure that inhibits or prevents crack propagation and/or fatigue failure of the refractory metal alloy.

Another and/or alternative non-limiting object of the present disclosure is the provision of a medical device that includes a refractory metal alloy having a nitriding process to form a nitrided layer on the outer surface of the refractory metal alloy.

Another and/or alternative non-limiting object of the present disclosure is the provision of a medical device that includes a refractory metal alloy wherein the refractory metal alloy has been subjected to a swaging process.

Another and/or alternative non-limiting object of the present disclosure is the provision of a medical device that includes a refractory metal alloy wherein the refractory metal alloy has been subjected to a cold-working process.

Another and/or alternative non-limiting object of the present disclosure is the provision of a medical device that includes a refractory metal alloy that has increased strength and/or hardness as compared with stainless steel, chromium-cobalt alloys, or titanium alloys.

Another and/or alternative non-limiting object of the present disclosure is the provision of a medical device that includes a refractory metal alloy thereby requiring a less quantity of refractory metal alloy to achieve similar strengths compared to medical devices formed of different metals.

Another and/or alternative non-limiting object of the present disclosure is the provision of a medical device that includes a refractory metal alloy wherein the medical device has a smaller crimped profile as compared to medical devices formed of different metals.

Another and/or alternative non-limiting object of the present disclosure is the provision of a medical device that includes a refractory metal alloy wherein the medical device has thinner walls and/or struts than in frames of a same shape that are formed of stainless steel, cobalt and chromium alloy or titanium alloy, and such frame formed of refractory metal alloy has the same or increase radial strength when the frame is expanded form a crimped configuration to an expanded configuration as compared to such frames formed of stainless steel or cobalt and chromium alloy, or titanium alloy.

Another and/or alternative non-limiting object of the present disclosure is the provision of a medical device that includes a refractory metal alloy wherein the medical device has improved stress-strain properties, bendability properties, elongation properties, and/or flexibility properties as compared to medical devices formed of stainless steel, titanium alloy, or chromium-cobalt alloys.

Another and/or alternative non-limiting object of the present disclosure is the provision of a medical device that includes a refractory metal alloy wherein the medical device has an increase life as compared to medical devices formed of stainless steel, titanium alloy, or chromium-cobalt alloys.

Another and/or alternative non-limiting object of the present disclosure is the provision of a medical device that includes a refractory metal alloy wherein the medical device has a reduced degree of recoil during the crimping and/or expansion of the medical device compared with frames of a similar size, shape and configuration that are formed of stainless steel, chromium-cobalt alloys, or titanium alloys.

Another and/or alternative non-limiting object of the present disclosure is the provision of a medical device that includes a refractory metal alloy wherein the medical device better conforms to an irregularly shaped body passageway when expanded in the body passageway as compared with frames of a similar size, shape and configuration that are formed of stainless steel, chromium-cobalt alloys, or titanium alloys.

Another and/or alternative non-limiting object of the present disclosure is the provision of a medical device that includes a refractory metal alloy wherein the medical device has improved fatigue ductility when subjected to cold-working as compared to the cold-working of frames of a similar size, shape and configuration that are formed of stainless steel, chromium-cobalt alloys, or titanium alloys.

Another and/or alternative non-limiting object of the present disclosure is the provision of a medical device that includes a refractory metal alloy wherein the medical device has improved durability as compared to stainless steel, chromium-cobalt alloys, or titanium alloys.

Another and/or alternative non-limiting object of the present disclosure is the provision of a medical device that includes a refractory metal alloy wherein the medical device has improved hydrophilicity as compared to stainless steel, chromium-cobalt alloys, or titanium alloys.

Another and/or alternative non-limiting object of the present disclosure is the provision of a medical device that includes a refractory metal alloy wherein the medical device has reduced ion release in the body passageway as compared to stainless steel, chromium-cobalt alloys, or titanium alloys.

Another and/or alternative non-limiting object of the present disclosure is the provision of a medical device that includes a refractory metal alloy wherein the medical device is less of an irritant to the body than stainless steel, cobalt-chromium alloy, or titanium alloys, thus can result in reduced inflammation, faster healing, and increased success rates of the medical device.

These and other advantages will become apparent to those skilled in the art upon the reading and following of this description.

### BRIEF DESCRIPTION OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. Reference may now be made to the drawings, which illustrate various embodiments that the disclosure may take in physical form and in certain parts and arrangement of parts wherein:

FIGS. 1A-1E are illustrations of a TAV, a portion of a catheter and a typical TAVR procedure for inserting the TAV into a valve of a heart.

FIG. 2A is an illustration of a TAV that includes an inner skirt and leaflet structure.

FIG. 2B is an illustration of a TAV frame.

FIGS. 3 and 4 are illustrations of a prior art TAV frame formed of CoCr alloy and a TAV frame in accordance with the present disclosure formed of refractory metal alloy of MoRe alloy and having a similar shape and configuration to the TAV frame formed of CoCr alloy, and which illustrates that the frame of the TAV formed of CoCr alloy requires multiple crimping cycles to fully crimp the TAV which can result increased incidence of leaflet damage, and the frame of the TAV formed of MoRe alloy only requires a single crimping cycle to fully crimp the TAV, thereby resulting in reduced incidence of leaflet damage during crimping, and also which results in a decrease crimped outer diameter as compared to a TAV formed of CoCr alloy, and which also illustrates that after each crimping procedure, the frame formed of CoCr alloy has a recoil of greater than 9% after each crimping cycle whereas the recoil of the frame of the refractory metal alloy has a recoil of less than 2% after each crimping cycle, and wherein the final crimped diameter of the frame formed of CoCr alloy after multiple crimping cycles is 24-27Fr and the final crimped diameter of the frame formed of refractory metal alloy after a single crimping cycle is 18-21 Fr.

FIG. 5 is a graph that illustrates the amount of recoil of several different metal alloys.

FIG. 6 is an illustration of a prior art TAV frame formed of CoCr alloy and a TAV frame in accordance with the present disclosure formed of refractory metal alloy, and which illustrates that the recoil after balloon expansion of the TAV frame formed of CoCr is greater than the recoil of the TAV frame formed of refractory metal alloy after balloon expansion, thus the effective orifice area after expansion of the TAV frame formed of refractory metal alloy is greater than the effective orifice area after expansion of the TAV frame formed of CoCr alloy.

FIG. 7 is an illustration that compares the conformability of a metal strip or wire formed of refractory metal to the shape of a die surface as compared to the conformity of a metal strip or wire of CoCr alloy on the same die surface.

FIGS. 8A and 8B are illustrations that compare the conformability of a TAV frame formed of refractory metal alloy that is expanded in a non-circular aortic valve that includes calcium deposits to a similar shaped and configured TAV frame formed of CoCr alloy that is expanded in the same non-circular aortic valve, and which illustrates that the paravalvular leak (PVL) about a TAV having a frame formed of CoCr alloy is greater than the PVL about a TAV having a frame formed of refractory metal alloy due the increase conformability of the frame formed of refractory metal alloy as compared to the conformability of the frame formed of CoCr alloy.

FIGS. 9A-9C illustrate stress vs. reduction in percent area graphs of TiAlV alloy, CoCr alloy and MoRe alloy.

FIG. 10 is a graph that illustrates the differences of stiffness and yield strength of a MoRe alloy, CoCr alloy and TiAlV alloy.

FIGS. 11-13 are graphs that illustrate the strength and fatigue ductility of a TiAlV alloy, CoCr alloy and MoRe alloy.

FIG. 14 illustrates the durability of a MoRe alloy compared to CoCr alloy and a TiAlV alloy.

FIG. 15 illustrates the purity of a MoRe alloy to a CoCr alloy and a TiAlV alloy.

FIG. 16 illustrates the hydrophilicity of a MoRe alloy, a CoCr alloy, and a TiAlV alloy.

FIGS. 17-18 illustrate the ion release rates of a refractory metal alloy such as MoRe.

FIG. 19 illustrates the ion release rates in tissue from a MoRe alloy, a CoCr alloy, and a TiAlV alloy.

### DESCRIPTION OF NON-LIMITING EMBODIMENTS OF THE DISCLOSURE

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

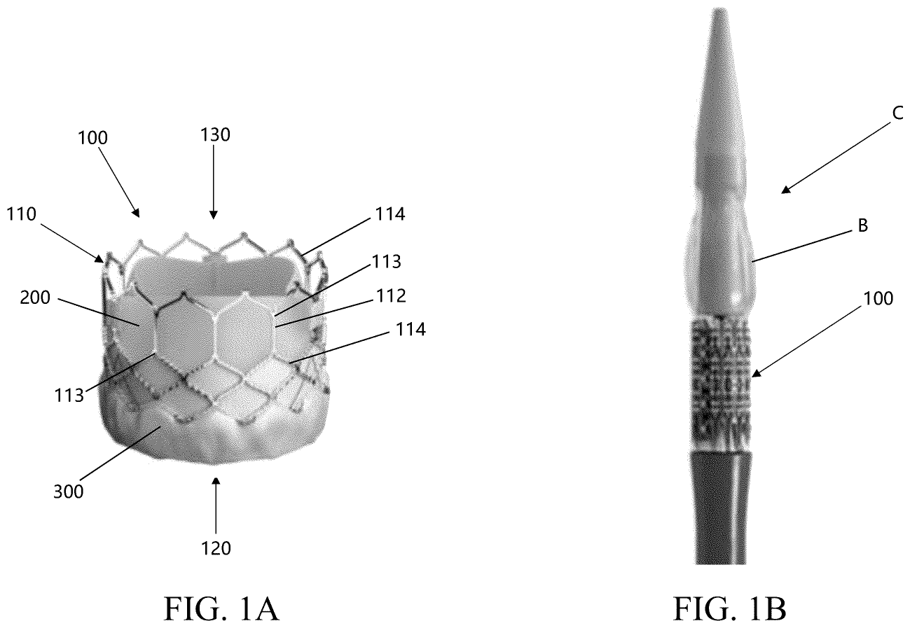

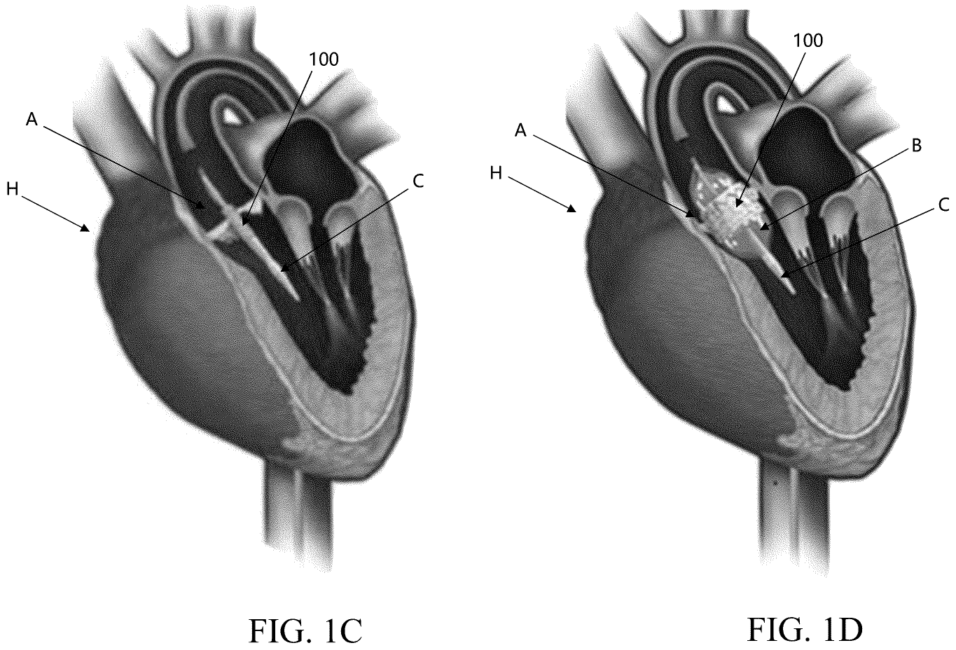

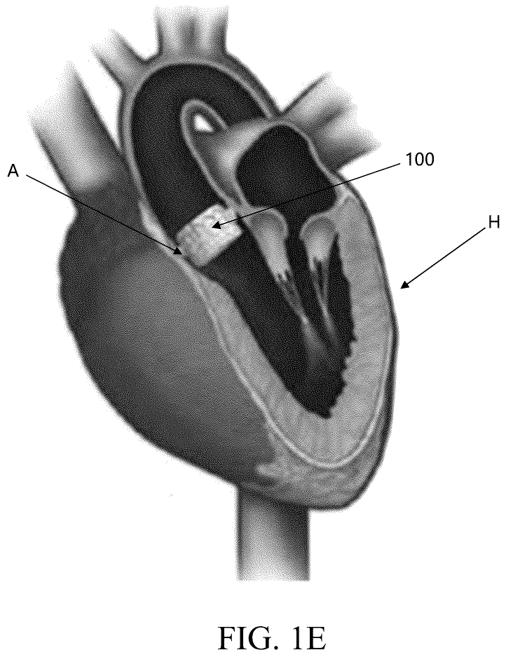

Referring now to FIGS. 1A-1E, these figures are illustrations of implantable prosthetic heart valve 100 (e.g., TAV) and a method for inserting the prosthetic heart valve 100 in a valve region A (e.g., aortic valve, etc.) of a heart H. The prosthetic heart valve 100 can be implanted in the annulus of the native aortic valve A; however, the prosthetic heart valve 100 also can be configured to be implanted in other valves of the heart. Although the medical device illustrated is a TAV, the present disclosure is not limited to TAVs or any other heart valve replacement. The medical device in accordance with the present disclosure can be any medical device that can be inserted or otherwise applied to a patient. Non-limiting medical devices in accordance with the present disclosure include orthopedic devices, PFO devices, stents, valves (e.g., heart valve, etc.), spinal implants, devices for treating aneurysms, occlusive devices for use in blood vessels and other body passageways, flow adjusting and/or diversion devices for blood vessels, devices for de-endothelializing a wall of an aneurysm, frame and other structures for use with a spinal implants, vascular implant, graft, dental implant, wire for used in medical procedures, bone implant; artificial disk, artificial spinal disk, prosthetic implant or device to repair, replace and/or support a bone and/or cartilage, bone plate, nail; rod, screw, post; cage, plate, pedicle screw, joint system, anchor, bone spacer, or disk that is used in a body to support a structure, mount a structure, and/or repair a structure in a body such as, but not limited to, a human body, animal body, etc.

Referring now to FIG. 1A, the prosthetic heart valve 100 generally comprises a frame 110 or stent formed of a plurality of posts and/or struts 112, 114 strut joints 113, leaflet structure 200 supported by the frame 110, and an inner skirt 300 secured to the outer surface of the frame 110 and/or leaflet structure 200. The prosthetic heart valve 100 has a “lower” end 120 and an “upper” end 130, wherein the lower end 120 of the prosthetic heart valve 100 is the inflow end and the upper end 130 of the prosthetic heart valve 100 is the outflow end.

The configuration of the frame 110 of the prosthetic heart valve 100 is non-limiting. Many different frame configurations can be used for the frame 110 of the prosthetic heart valve 100. The frame 110 includes a plurality of spaced, vertically extending struts or posts 112, or non-vertically extending struts 114 that are connected together at strut joints 113. As can be appreciated, the frame 110 can be fully formed of non-vertically extending struts 114 that are connected together at strut joints 113.

As illustrated in FIG. 1A, the frame 110 has a 12-post configuration wherein twelve vertically extending struts or post 112 are position about the upper portion of the frame 110. The vertically extending struts or posts 112 are interconnected via a lower row of circumferentially non-vertically extending struts 114 at strut joints 113 and an upper row of circumferentially non-vertically extending struts 114. The non-vertically extending struts 114 can be arrangement in a variety of patterns (e.g., zig-zag pattern, saw-tooth pattern, triangular pattern, polygonal pattern, oval pattern, S-shaped, Y-shaped, H-shaped, E-shaped, V-shaped, Z-shaped, L-shaped, J-sped, W-shaped, U-shaped, N-shaped, M-shaped, C-shaped, X-shaped, F-shaped, etc.). One or more of the posts and/or struts 112, 114 can have the same or different a) thicknesses, b) cross-sectional shape, and/or c) cross-sectional area along a portion or all of the longitudinal length.

As illustrated in FIG. 1A, the upper portion of the frame 110, when expanded, forms twelve hexagonal shaped structures on the upper portion of the frame 110, and three rings of quadrangle shaped structures that form the lower portion of the frame 110. The twelve hexagonal shaped structures are formed of a combination of vertically extending struts or post 112 and non-vertically extending struts 114, and the three rings of quadrangle shaped structures are formed only of non-vertically extending struts 114. As can be appreciated, many of the frame configurations can be used to form frame 110 for the prosthetic heart valve 100.

The frame 110 is partially or fully formed of a refractory metal alloy in accordance with the present disclosure. Non-limiting refractory metal alloys include a Re alloy (30-60 wt. % Re, 40-70 wt. % one or more metal additives [e.g., Mo, Bi, Nb, Ni, Ta, Ti, V, W, Mn, Zr, Ir, Tc, Ru, Rh, Hf, Os, Cu, Y); MoRe alloy, ReW alloy, MoReCr alloy, MoReTa alloy, MoReTi alloy, WCu alloy, ReCr alloy, Mo alloy, W alloy, Ta alloy, Nb alloy, etc. In one non-limiting embodiment, 90-100% of the frame 110 of the prosthetic heart valve 100 is formed of a refractory metal alloy. In one non-limiting example, 90-100% of the frame 110 of the prosthetic heart valve 100 is formed of MoRe alloy (e.g., 40-60 wt. % Mo, 40-60 wt. % Re and 0-10 wt. % of one or more other metal additives).

The frame 110 can be optionally be coated with a polymer material (e.g., silicone, PTFE, ePTFE, polyurethane, polyolefins, hydrogels, biological materials [e.g., pericardium or biological polymers such as collagen, gelatin, or hyaluronic acid derivatives], etc.). The coating can be used to partially or fully encapsulate one or more of the vertically extending struts or posts 112 and/or non-vertically extending struts 114 on the frame 110 and/or to partially of fully fill-in one or more of the openings between the non-vertically extending struts 114 and/or vertically extending struts or posts 112.

The inner skirt 300 can be formed of a variety of flexible materials (e.g., polymer (e.g., polyethylene terephthalate (PET), polyester, nylon, Kevlar, silicon, etc.), composite material, metal, fabric material, etc. In one non-limiting embodiment, the material used to partially or fully form the inner skirt 300 can be substantially non-elastic (i.e., substantially non-stretchable and non-compressible). In another non-limiting embodiment, the material used to partially or fully form the inner skirt 300 can be a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The inner skirt 300 can optionally be formed from a combination of a cloth or fabric material that is coated with a flexible material or with a stretchable and/or compressible material so as to provide additional structural integrity to the inner skirt 300. The size, configuration and thickness of the inner skirt 300 is non-limiting (e.g., thickness of 0.1-20 mils and all values and ranges therebetween). The inner skirt 300 can be secured to the inside and/or outside of the frame 110 using various means (e.g., sutures, clips, clamp arrangement, etc.).

The inner skirt 300 can be used to 1) at least partially seal and/or prevent perivalvular leakage, 2) at least partially secure the leaflet structure 200 to the frame 110, 3) at least partially protect one or more of the leaflets of the leaflet structure 200 from damage during the crimping process of the prosthetic heat valve 100, 4) at least partially protect one or more of the leaflets of the leaflet structure 200 form damage during the operation of the prosthetic heart valve 100 in the heart H.

The prosthetic heart valve 100 can optionally include an outer skirt or sleeve (not shown) that is positioned at least partially about the exterior region of the frame 110. The outer skirt or sleeve, when used, generally is positioned completely around a portion of the outside of the frame 110. Generally, the outer skirt is positioned about the lower portion of the frame 110 and does not fully cover the upper portion of the frame 110; however, this is not required. The outer skirt can be connected to the frame 110 by a variety of arrangements (e.g., sutures, adhesive, melted connection, clamping arrangement, etc.). At least a portion of the outer skirt can optionally be located on the interior surface of the frame 110; however, this is not required. Generally, the outer skirt is formed of a more flexible and/or compressible material than the inner skirt 300; however, this is not required. The outer skirt can be formed of a variety of a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The outer skirt can optionally be formed from a combination of a cloth or fabric material that is coated with the stretchable and/or compressible material so as to provide additional structural integrity to the outer skirt. The size, configuration and thickness of the outer skirt is non-limiting. The thickness of the outer skirt is generally 0.1-20 mils (and all values and ranges therebetween).

The leaflet structure 200 can be can be attached to the frame 110 and/or inner skirt 300. The connection arrangement used to secure the leaflet structure 200 to the frame 110 and/or inner skirt 300 is non-limiting (e.g., sutures, melted bold, adhesive, clamp arrangement, etc.). The material used to form the one or more leaflets of the leaflet structure 200 include, but are not limited to, bovine pericardial tissue, biocompatible synthetic materials, or various other suitable natural or synthetic materials.

The leaflet structure 200 can be comprised of two or more leaflets (e.g., 2, 3, 4, 5, 6, etc.). In one non-limiting arrangement, the leaflet structure 200 includes three leaflets that are arranged to collapse in a tricuspid arrangement. The size, shape and configuration of the one or more leaflets of the leaflet structure 200 are non-limiting. In one non-limiting arrangement, the leaflets have generally the same shape, size, configuration and thickness.

Two of more of the leaflets of the leaflet structure 200 can optionally be secured to one another at their adjacent sides to form commissures of the leaflet structure 200 (the edges where the leaflets come together). The leaflet structure 200 can be secured to the frame 110 and/or inner skirt 300 by a variety of connection arrangement (e.g., sutures, adhesive, melted bond, clamping arrangement, etc.).

One or more leaflets of the leaflet structure 200 can optionally include reinforcing structures or strips to 1) facilitate in securing the leaflets together, 2) facilitate in securing the leaflets to the inner skirt 300 and/or frame 110, and/or 3) inhibit or prevent tearing or other types of damage to the leaflets.

The prosthetic heart valve 100 is configured to be radially collapsible to a collapsed or crimped state for introduction into the body on a delivery catheter (FIG. 1B) and radially expandable to an expanded state for implanting the prosthetic heart valve 100 at a desired location in the heart H (e.g., the aortic valve A, etc.) (FIG. 1E). The frame 110 of the prosthetic heart valve 100 is made of a plastically-expandable material (e.g., refractory metal alloy) that permits crimping of the frame 110 to a smaller profile for delivery and expansion of the prosthetic heart valve 100 using an expansion device such as the balloon B of a balloon catheter C.

Referring now to FIG. 1B, the prosthetic heart valve 100 is crimped into a portion of balloon B of the balloon catheter C. Various type of crimping apparatus and techniques can be used to crimp the prosthetic heart valve on the balloon delivery catheter. The process of crimping a prosthetic heart valve 100 using a crimping device is known in the art and will not be described herein. During a crimping procedure, damage to the leaflets of leaflet structure 200 should be avoided.

As illustrated in FIGS. 1C-1E, once the prosthetic heart valve 100 is crimped on the balloon B of a balloon delivery catheter C, the balloon delivery catheter C is inserted through a blood vessel and to the location in the heart H wherein the prosthetic heart valve 100 is to be deployed (See FIG. 1C). At the treatment location, the balloon B on the balloon delivery catheter C is expanded to thereby cause the prosthetic heart valve 100 to be expanded and secured in a valve region A of the heart H (See FIG. 1D). Thereafter, the balloon B is deflated and the balloon delivery catheter C is removed from the patient (See FIG. 1E).

The frame 110 of the prosthetic heart valve 100 can be configured such that it can be crimped onto a delivery catheter C so that the crimped prosthetic heart valve 100 can be inserted in heart valves that are less than 22 Fr. Commercially available prior art prosthetic heart values can only be crimped to a diameter of about 24-27 FR (8-9 mm) due to the materials used to form the frame of such prosthetic heart valves. As such, the prosthetic heart valve 100 in accordance with the present disclosure can be inserted into smaller sized heart valves that could not previously be treated with prior art prosthetic heart valves. As can be appreciated, the prosthetic heart valve 100 in accordance with the present disclosure can be sized and configured to be inserted in heart valves that are greater than 22 Fr.

The refractory metal alloy frame 110 of the prosthetic heart valve 100 and other types of expandable medical devices (e.g., stents, etc.) can be crimped to have a crimped outer diameter that is at least 5% and up to a 33% smaller (e.g., 5-33% smaller and all value and ranges therebetween) than a crimped outer diameter of a frame of the same size, configuration and shape that is formed of Co—Cr alloy (e.g., L605; MP35N; Phynox; Eligory; 35Co-35Ni-20Cr-10Mo; 40Co-20Cr-16Fe-15Ni7Mo; Co-20Cr-15W-10Ni; 15-30 wt. % Cr, 10-20 wt. % W, 5-35 wt. % Ni, 0-3 wt. % Fe, 0-2 wt. % Mn, 0-10 wt. % Mo, 0-1 wt. % Ti, 0.0.5 wt. % Si).

The refractory metal alloy frame 110 of the prosthetic heart valve 100 and other types of expandable medical devices (e.g., stents, etc.) can be crimped to have a crimped outer diameter that is at least 5% and up to a 50% smaller (e.g., 5-50% smaller and all value and ranges therebetween) than a crimped outer diameter of a frame of the same size, configuration and shape that is formed of stainless steel (e.g., 316, 316L). In the medical industry, expandable frames for prosthetic heart valves are only formed of certain cobalt-chromium alloys and NiTi alloys. Stainless steel and other alloys such as TiAlV alloys, that can be used in other types of expandable medical devices (e.g., stents, etc.), are not used for prosthetic heart valves for various reasons. Although the present disclosure illustrates the many advantages for using a refractory metal alloy in accordance with the present disclosure in expandable medical devices, comparisons of the refractory metal alloy of the present disclosure to cobalt-chromium alloys and NiTi alloys will only be made in this disclosure when referring to expandable prosthetic heart valves such as, but not limited to TAVR devices.

The refractory metal alloy frame 110 of the prosthetic heart valve 100 and other types of expandable medical devices (e.g., stents, etc.) can be crimped to have a crimped outer diameter that is at least 5% and up to a 40% smaller (e.g., 5-40% smaller and all value and ranges therebetween) than a crimped outer diameter of a frame of the same size, configuration and shape that is formed of nitinol (self-expanding nickel titanium alloy—49-60% wt. % Ni and 40-51 wt. % Ti).

The refractory metal alloy frame 110 of the prosthetic heart valve 100 and other types of expandable medical devices (e.g., stents, etc.) can be crimped to have a crimped outer diameter that is at least 5% and up to a 40% smaller (e.g., 5-40% smaller and all value and ranges therebetween) than a crimped outer diameter of a frame of the same size, configuration and shape that is formed of TiAlV alloys (e.g., Ti-6Al-4V; 5.5-6.5 wt. % Al, 3.5-4.5 wt. % V and balance Ti; 3.5-4.5 wt. % vanadium, 5.5-6.75 wt. % aluminum, 0.3 wt. % max iron, 0.2 wt. % max oxygen, 0.08 wt. % max carbon, 0.05 wt. % max nitrogen, 0.015 wt. % max hydrogen H, 0.05 wt. % max yttrium, balance titanium).

As such, outer crimp diameters of the prosthetic heart valve 100 of 6-7 mm (18-21 FR) for a TAVR that was designed to be expanded to have an effective orifice area (EOA) of at least 585 mm², which were previously unachievable using stainless steel, CoCr alloy, nitinol, and can now be obtained by using a frame 110 formed of a refractory metal in accordance with the present disclosure.

Most commercially available expandable frames used for prosthetic heart valves are formed of cobalt-chromium alloy (e.g., MP35N, etc.) or NiTi alloy (e.g., Nitinol, etc.). Frames formed of stainless steel for prosthetic heart valves have fallen out of favor due to the problems associated with the corrosion of the stainless steel and the required larger strut thicknesses required to form a frame strong enough for use in a heart valve. Frames for expandable medical devices such as stents are still sometimes formed of stainless steel and other alloys such as TiAlV alloys.

As illustrated in Table 1, a frame for a prosthetic heart device (e.g., TAVR, etc.) that is formed of a refractory metal alloy in accordance with the present disclosure has improved properties as compared to frames for prosthetic heart valves that are formed of MP35N or NiTi alloy. As illustrated below, for a frame of a prosthetic heart valve that is designed to expand to 25 mm, the frame of the prosthetic heart valve that is formed of a refractory metal alloy (e.g., MoRe, etc.) has several advantages over frames for prosthetic heart valves that are formed of MP35N or NiTi alloy, namely 1) the outer diameter (OD) of the crimped prosthetic valve having a frame formed of refractory metal alloy is smaller than the OD crimped diameter of the crimped prosthetic valve having a frame formed of MP35N (e.g., cobalt-chromium alloy) or NiTi alloy, 2) the strut joint width on the frame (e.g., the location that the end of a strut is connected to another portion of the frame) that is formed of a refractory metal alloy can be less than the strut joint width on the frame formed of MP35N or NiTi alloy, 3) the strut width on the frame that is formed of a refractory metal alloy is less than the strut width on the frame formed of MP35N or NiTi alloy, 4) the amount of recoil of a frame that is formed of a refractory metal alloy after the frame has been crimped or after the frame has been expanded is less than the amount of recoil of a frame that is formed of a MP35N or NiTi alloy after such frame has been crimped or after the frame has been expanded, and 5) the amount of foreshortening of a frame that is formed of a refractory metal alloy after the frame has been expanded is less than the amount of foreshortening of a frame that is formed of a MP35N or NiTi alloy after such frame has been expanded.

The strength of the refractory metal alloy is greater than a cobalt-chromium alloy, stainless steel, nickel-titanium alloy or a TiAlV alloy, thus the strut width and strut joints of expandable frames can be made smaller than frames formed of such other alloys, thereby a frame for a medical formed of a refractory metal alloy can be made smaller without sacrificing the strength of the frame. The amount of recoil of a frame formed of refractory metal alloy when the frame is crimped or expanded from the crimped state is less than the amount of recoil of a frame formed of cobalt-chromium alloy, stainless steel, nickel-titanium alloy or a TiAlV alloy. As such, a reduced number of crimping cycles (typically only one crimping cycle is required) is needed to obtain the desired crimped profile of the frame as compared to multiple crimped cycles are required to obtain the desired crimped profile of the frame that are formed of cobalt-chromium alloy, stainless steel, nickel-titanium alloy or a TiAlV alloy. The amount of recoil of a frame formed of a refractory metal alloy when the frame is crimped or when the frame is expanded from a crimped stated is generally no more than 8% (e.g., 0-8% and all values and ranges therebetween), typically no more than 5%, more typically no more than 3%, still more typically no more than 2%, and even more typically less than 2%. The amount of foreshortening of a frame formed of refractory metal alloy when the frame is expanded from a crimped state is less than the amount of foreshortening of a frame formed of cobalt-chromium alloy, stainless steel, nickel-titanium alloy or a TiAlV alloy. Generally the amount of foreshortening of a frame formed of a refractory metal alloy from a crimped state to an expanded state is 0-20% (and all values and ranges therebetween), typically 0-15%, more typically 0-10%, and still more typically 0-5%. As such, a reduced amount of foreshortening facilitates in ensuring the medical device when the frame is expanded from the crimped state maintains its proper position in the treatment area. Frames of medical devices that foreshorten reduce in longitudinal length when the frame is expanded. Such reduction in longitudinal length during expansion of the frame can result in the mis-location of the expanded device in a treatment area, which mis-location can result in a) improper operation of the implanted medical device, b) damage to the implanted medical device, c) potential damage to the tissue about the implanted medical device, d) reduced life of the medical device, e) causing plaque and/or calcium deposits to form about the medical device, etc.

TABLE 1
TABLE 1

| | | Expanded | | | | | | |
|---|---|---|---|---|---|---|---|---|
| | | OD (for 25 | | | | | | |
| | | mm valve, | | | | | | |
| | | this is size | | Radial | | | | |
| | | specific and | | Strength | Strut | | | |
| | Crimped OD | material | | after | Joint | Strut | | |
| | of the Valve | independent) | Maximum | Expansion | Width | Width | % | % |
| Frame alloy | (average mm) | (mm) | EOA | (N) | (mm) | (mm) | Recoil | Foreshortening |
| MoRe | <7 | 23-27 | 2.8 | 35 | 0.3-0.7 | 0.3 | <2 | 0 |
| (52.5% Mo- | | | | | | | | |
| 47.5% Re) | | | | | | | | |
| MP35N | 7.7 | 26 | 2.45 | 35 | 0.7 | 0.3 | >5 | 30 |
| NiTi | 7.7 | 25 at | 3 | 35 | 0.85 | 0.33 | >5 | 20 |
| (Nitinol - | | sufficient | | | | | | |
| 49-60% | | outward | | | | | | |
| wt. % Ni | | force to | | | | | | |
| and 40-51 | | maintain | | | | | | |
| wt. % Ti) | | position | | | | | | |

Table 1 illustrates that for a frame formed of a non-self-expanding metal alloy for use in a prosthetic heat valve, the frame formed of a refractory metal alloy in accordance with the present invention has superior properties to frame formed of cobalt chromium alloy with regard to crimped OD, maximum EOA, strut joint width, % recoil, and % foreshortening. It is noted that even for self-expanding frames that are formed of Nitinol, frame formed of refractory metal alloy in accordance with the present invention has superior properties to frame formed of Nitinol with regard to crimped OD, strut joint width, % recoil, and % foreshortening. Due to the self-expanding nature of Nitinol, the Nitinol frame has improved EOA as compared to the frame formed of refractory metal alloy.

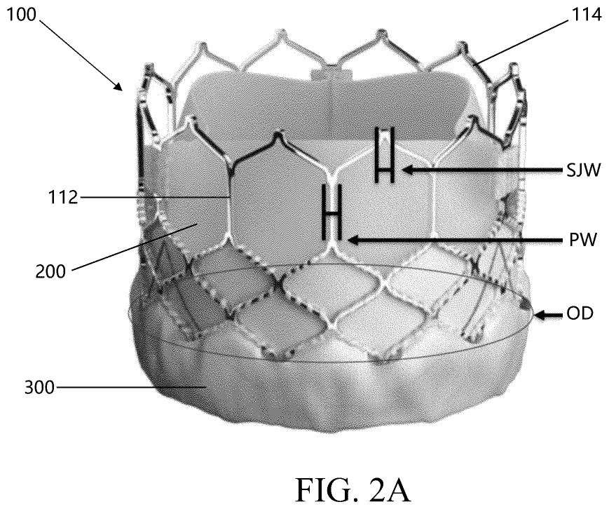

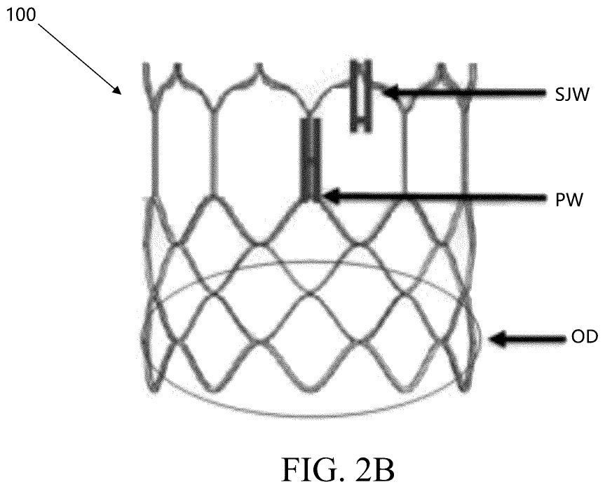

Referring now to FIGS. 2A-2B, the post width PW and/or the strut joint width SJW of a frame 110 that is formed of a refractory metal alloy in accordance with the present disclosure can be smaller than the post width PW and/or the strut joint width SJW of a frame formed of stainless steel, nitinol, Co—Cr alloy or TiAlV alloy, and still have the same or greater radial strength when the frame is expanded as compared to a frame formed of stainless steel, nitinol, Co—Cr alloy or TiAlV alloy.

Generally, refractory metal alloys (e.g., 30-60 wt. % Re, 40-70 wt. % one or more metal additives [e.g., Mo, Bi, Nb, Ni, Ta, Ti, V, W, Mn, Zr, Ir, Tc, Ru, Rh, Hf, Os, Cu, Y]; MoRe alloy, ReW alloy, MoReCr alloy, MoReTa alloy, MoReTi alloy, WCu alloy, ReCr alloy, Mo alloy, Re alloy, W alloy, Ta alloy, Nb alloy, etc.) used to form struts and posts and strut joint on expandable frames of TAV frames have an average cross-sectional area that is 5-40% (and all values and ranges therebetween) less than the cross-sectional area of the struts and posts and strut joints on TAV frames formed of CoCr alloy (e.g., L605; MP35N), and wherein such refractory metal alloy struts, posts and strut joints have the same or greater strength than such struts, posts and strut joints formed of CoCr alloy.

Generally, refractory metal alloys (e.g., 30-60 wt. % Re, 40-70 wt. % one or more metal additives [e.g., Mo, Bi, Nb, Ni, Ta, Ti, V, W, Mn, Zr, Ir, Tc, Ru, Rh, Hf, Os, Cu, Y]; MoRe alloy, ReW alloy, MoReCr alloy, MoReTa alloy, MoReTi alloy, WCu alloy, ReCr alloy, Mo alloy, Re alloy, W alloy, Ta alloy, Nb alloy, etc.) used to form struts and posts and strut joint on expandable frames of TAV frames have an average cross-sectional area that is 5-50% (and all values and ranges therebetween) less than the cross-sectional area of less than the cross-sectional area of the struts and posts and strut joints on TAV frames formed of stainless steel (e.g., 316, 316L), and wherein such refractory metal alloy struts, posts and strut joints have the same or greater strength than such struts, posts and strut joints formed of stainless steel.

Generally, refractory metal alloys (e.g., 30-60 wt. % Re, 40-70 wt. % one or more metal additives [e.g., Mo, Bi, Nb, Ni, Ta, Ti, V, W, Mn, Zr, Ir, Tc, Ru, Rh, Hf, Os, Cu, Y]; MoRe alloy, ReW alloy, MoReCr alloy, MoReTa alloy, MoReTi alloy, WCu alloy, ReCr alloy, Mo alloy, Re alloy, W alloy, Ta alloy, Nb alloy, etc.) used to form struts and posts and strut joint on expandable frames of TAV frames have an average cross-sectional area that is 5-40% (and all values and ranges therebetween) less than the cross-sectional area of the struts and posts and strut joints on TAV frames formed of TiAlV alloy (e.g., Ti-6Al-4V), and wherein such refractory metal alloy struts, posts and strut joints have the same or greater strength than such struts, posts and strut joints formed of TiAlV alloy.

Generally, refractory metal alloys (e.g., 30-60 wt. % Re, 40-70 wt. % one or more metal additives [e.g., Mo, Bi, Nb, Ni, Ta, Ti, V, W, Mn, Zr, Ir, Tc, Ru, Rh, Hf, Os, Cu, Y]; MoRe alloy, ReW alloy, MoReCr alloy, MoReTa alloy, MoReTi alloy, WCu alloy, ReCr alloy, Mo alloy, Re alloy, W alloy, Ta alloy, Nb alloy, etc.) used to form struts and posts and strut joint on expandable frames of TAV frames have an average cross-sectional area that is 5-40% (and all values and ranges therebetween) less than the cross-sectional area of the struts and posts and strut joints on TAV frames formed of Nitinol (e.g., 50-60 wt. % Ni and 40-60 wt. % Ti), and wherein such refractory metal alloy struts, posts and strut joints have the same or greater strength than such struts, posts and strut joints formed of Nitinol.

The use of the refractory metal alloy to form the frame of TAV frames allows for smaller expandable TAVs to be manufactured. Such smaller expandable TAVs can be inserted into smaller blood vessels or other body passageways that previously could not be accessed using TAVs formed of other types of metal alloys.

### Example 1

There is provided one frame for a prosthetic heart valve formed of a refractory alloy such a MoRe (e.g., 40-60 wt. % Mo, 40-60 wt. % Re, 0-10 wt. % one or more metal additives) and another frame for a prosthetic heart valve formed of CoCr alloy (35Co-35Ni-20Cr-10Mo)—[wherein a) both frames have the same number of posts and struts, b) both frames have the same post and strut configuration and shape, c) both frames can be expanded to create an effective orifice area (EOA) of at least 585 mm², d) both frames have the same frame shape and configuration, and e) the frame formed of MoRe has the same or greater radial strength in an expanded stated as the frame formed of CoCr in an expanded state]—, the frame that is formed of MoRe can have both thinner SJWs (0.5 mm) and PWs (0.2 mm) than the SJWs (0.7 mm) and PWs (0.3 mm) of the frame formed of CoCr alloy. Because the SJWs and PWs for the frame formed of MoRe alloy are smaller than the SJWs and PWs of the frame formed of CoCr alloy, I) the amount of material required to form the frame of MoRe alloy is less than the amount of material required to form of the frame of CoCr alloy, and II) the frame formed of MoRe can be crimped to have an outer crimped diameter (6-7 mm or 18-21 Fr) that is less (up to 33+% less) than the outer crimped diameter of the frame formed of CoCr (8-9 mm or 24-27 Fr), which frame is used to form a EOA of at least 585 mm².

Due to the properties of the refractory metal alloy, a TAV frame formed of refractory metal alloy as compared to a TAV frame formed of CoCr alloy [wherein both frames have a) the same number of posts and struts, b) the same post and strut configuration and shape, c) the frame formed of refractory metal alloy can be expanded to the same or greater effective orifice area (EOA) as the expanded frame formed of CoCr, and d) the frame formed of MoRe has the same or greater radial strength in an expanded state as the frame formed of CoCr in an expanded state] can 1) be formed of at least 5% less material (e.g., 5-35% less material and all values and ranges therebetween), 2) have SJWs that are at least 5% thinner (e.g., 5-35% thinner and all values and ranges therebetween), c) have PWs that are at least 5% thinner (e.g., 5-35% thinner and all values and ranges therebetween), and 3) have a crimped outer diameter that is at least 5% smaller (e.g., 5-22.2% smaller and all values and ranges therebetween).

The post width and strut joint width using a refractory metal alloy can thus be reduced as compared to post widths and strut joint widths on frames formed of CoCr alloy without a reduction in the strength of the frame in the expanded state. Due to the smaller crimped outer diameter of the frame that is achievable using a refractory metal alloy, the outer crimped diameter of the prosthetic heart valve using a frame formed of refractory metal alloy is less than a prosthetic heart valve using a frame formed of CoCr alloy.

### Example 2

There is provided one frame for a prosthetic heart valve formed of a refractory alloy such a MoRe (e.g., 40-60 wt. % Mo, 40-60 wt. % Re, 0-10 wt. % one or more metal additives) and another frame for a prosthetic heart valve formed of stainless steel (e.g., 316 [16-19 wt. % Cr, 10-14 wt. % Ni, 2-3 wt. % Mo, less than 2 wt. % Mn, less than 1 wt. % Si, up to 0.08 wt. % C, and balance Fe], 316L [16-19 wt. % Cr, 10-14 wt. % Ni, 2-3 wt. % Mo, less than 2 wt. % Mn, less than 1 wt. % Si, up to 0.05 wt. % C, and balance Fe])—[wherein a) both frames have the same number of posts and struts, b) both frames have the same post and strut configuration and shape, c) both frames can be expanded to create an effective orifice area (EOA) of at least 585 mm², d) both frames have the same frame shape and configuration, and e) the frame formed of MoRe has the same or greater radial strength in an expanded stated as the frame formed of stainless steel in an expanded state]—, the frame that is formed of MoRe can have both thinner SJWs (0.5 mm) and PWs (0.2 mm) than the SJWs (0.8 mm) and PWs (0.4 mm) of the frame formed of stainless steel. Because the SJWs and PWs for the frame formed of MoRe alloy are smaller than the SJWs and PWs of the frame formed of stainless steel, I) the amount of material required to form the frame of MoRe alloy is less than the amount of material required to form of the frame of stainless steel, and II) the frame formed of MoRe can be crimped to have an outer crimped diameter (6-7 mm or 18-21 Fr) that is less (up to 50+% less) than the outer crimped diameter of the frame formed of stainless steel (9-10 mm or 27-30 Fr), which frame is used to form a EOA of at least 585 mm².

Due to the properties of the refractory metal alloy, a TAV frame formed of refractory metal alloy as compared to a TAV frame formed of stainless steel [wherein both frames have a) the same number of posts and struts, b) the same post and strut configuration and shape, c) the frame formed of refractory metal alloy can be expanded to the same or greater effective orifice area (EOA) as the expanded frame formed of stainless steel, and d) the frame formed of MoRe has the same or greater radial strength in an expanded state as the frame formed of stainless steel in an expanded state] can 1) be formed of at least 5% less material (e.g., 5-50% less material and all values and ranges therebetween), 2) have SJWs that are at least 5% thinner (e.g., 5-50% thinner and all values and ranges therebetween), c) have PWs that are at least 5% thinner (e.g., 5-50% thinner and all values and ranges therebetween), and 3) have a crimped outer diameter that is at least 5% smaller (e.g., 5-40% smaller and all values and ranges therebetween).

The post width and strut joint width using a refractory metal alloy can thus be reduced as compared to post widths and strut joint widths on frames formed of stainless steel without a reduction in the strength of the frame in the expanded state. Due to the smaller crimped outer diameter of the frame that is achievable using a refractory metal alloy, the outer crimped diameter of the prosthetic heart valve using a frame formed of refractory metal alloy is less than a prosthetic heart valve using a frame formed of stainless steel.

### Example 3

There is provided one frame for a prosthetic heart valve formed of a refractory alloy such a MoRe (e.g., 40-60 wt. % Mo, 40-60 wt. % Re, 0-10 wt. % one or more metal additives) and another frame for a prosthetic heart valve formed of Nitinol (e.g., 50-60 wt. % Ni and 40-60 wt. % Ti)—[wherein a) both frames have the same number of posts and struts, b) both frames have the same post and strut configuration and shape, c) both frames can be expanded to create an effective orifice area (EOA) of at least 585 mm², d) both frames have the same frame shape and configuration, and e) the frame formed of MoRe has the same or greater radial strength in an expanded stated as the frame formed of Nitinol in an expanded state]—, the frame that is formed of MoRe can have both thinner SJWs (0.5 mm) and PWs (0.2 mm) than the SJWs (0.7 mm) and PWs (0.3 mm) of the frame formed of Nitinol. Because the SJWs and PWs for the frame formed of MoRe alloy are smaller than the SJWs and PWs of the frame formed of Nitinol, I) the amount of material required to form the frame of MoRe alloy is less than the amount of material required to form of the frame of Nitinol, and II) the frame formed of MoRe can be crimped to have an outer crimped diameter (6-7 mm or 18-21 Fr) that is less (up to 50+% less) than the outer crimped diameter of the frame formed of Nitinol (8-9 mm or 24-27 Fr), which frame is used to form a EOA of at least 585 mm².

Due to the properties of the refractory metal alloy, a TAV frame formed of refractory metal alloy as compared to a TAV frame formed of Nitinol [wherein both frames have a) the same number of posts and struts, b) the same post and strut configuration and shape, c) the frame formed of refractory metal alloy can be expanded to the same or greater effective orifice area (EOA) as the expanded frame formed of Nitinol, and d) the frame formed of MoRe has the same or greater radial strength in an expanded state as the frame formed of Nitinol in an expanded state] can 1) be formed of at least 5% less material (e.g., 5-50% less material and all values and ranges therebetween), 2) have SJWs that are at least 5% thinner (e.g., 5-50% thinner and all values and ranges therebetween), c) have PWs that are at least 5% thinner (e.g., 5-50% thinner and all values and ranges therebetween), and 3) have a crimped outer diameter that is at least 5% smaller (e.g., 5-40% smaller and all values and ranges therebetween).

The post width and strut joint width using a refractory metal alloy can thus be reduced as compared to post widths and strut joint widths on frames formed of Nitinol without a reduction in the strength of the frame in the expanded state. Due to the smaller crimped outer diameter of the frame that is achievable using a refractory metal alloy, the outer crimped diameter of the prosthetic heart valve using a frame formed of refractory metal alloy is less than a prosthetic heart valve using a frame formed of Nitinol.

### Example 4

There is provided one frame for a prosthetic heart valve formed of a refractory alloy such a MoRe (e.g., 40-60 wt. % Mo, 40-60 wt. % Re, 0-10 wt. % one or more metal additives) and another frame for a prosthetic heart valve formed of TiAlV alloy (e.g., Ti-6Al-4V)—[wherein a) both frames have the same number of posts and struts, b) both frames have the same post and strut configuration and shape, c) both frames can be expanded to create an effective orifice area (EOA) of at least 585 mm², d) both frames have the same frame shape and configuration, and e) the frame formed of MoRe has the same or greater radial strength in an expanded stated as the frame formed of TiAlV alloy in an expanded state]—, the frame that is formed of MoRe can have both thinner SJWs (0.5 mm) and PWs (0.2 mm) than the SJWs (0.7 mm) and PWs (0.3 mm) of the frame formed of TiAlV alloy. Because the SJWs and PWs for the frame formed of MoRe alloy are smaller than the SJWs and PWs of the frame formed of TiAlV alloy, I) the amount of material required to form the frame of MoRe alloy is less than the amount of material required to form of the frame of TiAlV alloy, and II) the frame formed of MoRe can be crimped to have an outer crimped diameter (6-7 mm or 18-21 Fr) that is less (up to 50+% less) than the outer crimped diameter of the frame formed of TiAlV alloy (8-9 mm or 24-27 Fr), which frame is used to form a EOA of at least 585 mm².

Due to the properties of the refractory metal alloy, a TAV frame formed of refractory metal alloy as compared to a TAV frame formed of TiAlV alloy [wherein both frames have a) the same number of posts and struts, b) the same post and strut configuration and shape, c) the frame formed of refractory metal alloy can be expanded to the same or greater effective orifice area (EOA) as the expanded frame formed of TiAlV alloy, and d) the frame formed of MoRe has the same or greater radial strength in an expanded state as the frame formed of TiAlV alloy in an expanded state] can 1) be formed of at least 5% less material (e.g., 5-50% less material and all values and ranges therebetween), 2) have SJWs that are at least 5% thinner (e.g., 5-50% thinner and all values and ranges therebetween), c) have PWs that are at least 5% thinner (e.g., 5-50% thinner and all values and ranges therebetween), and 3) have a crimped outer diameter that is at least 5% smaller (e.g., 5-40% smaller and all values and ranges therebetween).

The post width and strut joint width using a refractory metal alloy can thus be reduced as compared to post widths and strut joint widths on frames formed of TiAlV alloy without a reduction in the strength of the frame in the expanded state. Due to the smaller crimped outer diameter of the frame that is achievable using a refractory metal alloy, the outer crimped diameter of the prosthetic heart valve using a frame formed of refractory metal alloy is less than a prosthetic heart valve using a frame formed of TiAlV alloy.

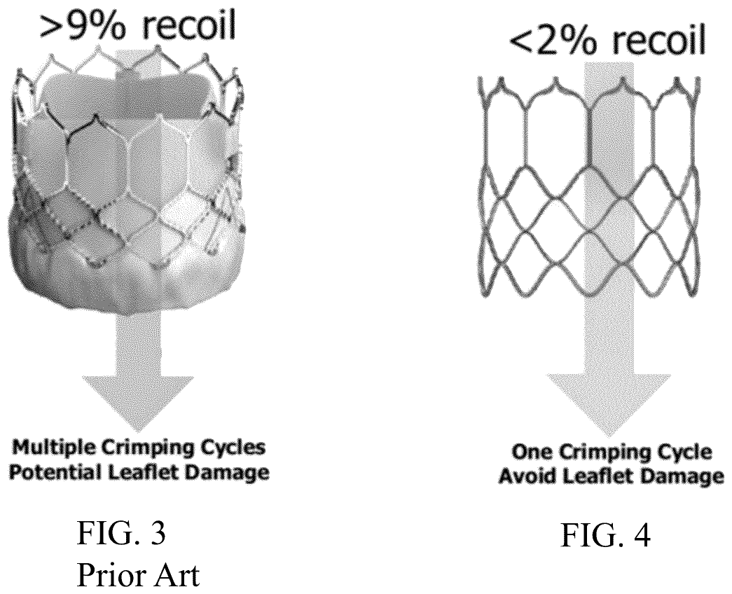

Referring now to FIGS. 3-4, one of the advantages of forming a frame of a prosthetic heart valve using a refractory metal alloy such as Mo—Re alloy is that refractory metal alloy exhibits less recoil when both crimped and expanded.

During the crimping of a TAV frame, the metal alloy used to form the TAV frame will recoil after the radial crimping forces are removed from the TAV frame. Likewise, when the crimped TAV frame is expanded, the expanded TAV frame will recoil to a smaller outer diameter after the expansion forces (e.g., expansion force from the inflation of a balloon on a catheter, etc.) on the TAV frame are removed.

As illustrated in FIG. 4, the crimping of a TAV frame formed of a refractory metal alloy such as (e.g., 40-60 wt. % Mo, 40-60 wt. % Re, 0-10 wt. % one or more metal additives) will recoil and expand by less than 2% (e.g., 0.1-2% and all values and ranges therebetween) after the radial crimping forces are removed from the frame. Generally, TAV frames formed of refractory metal alloys will recoil 0.1-5% (and all values and ranges therebetween) when subjected to a single crimping cycle, typically TAV frames formed of refractory metal alloys will recoil less than 2% when subjected to a single crimping cycle, and more typically TAV frames formed of refractory metal alloys will recoil about 0.1-1.95% when subjected to a single crimping cycle. Due to the low amount of recoil, the TAV frame only needs to be subjected to a single crimping cycle to obtain the smallest crimping outer diameter of the crimped frame. Also, such TAV frames can be crimped to smaller outer diameters than TAV frames formed of CoCr alloy.

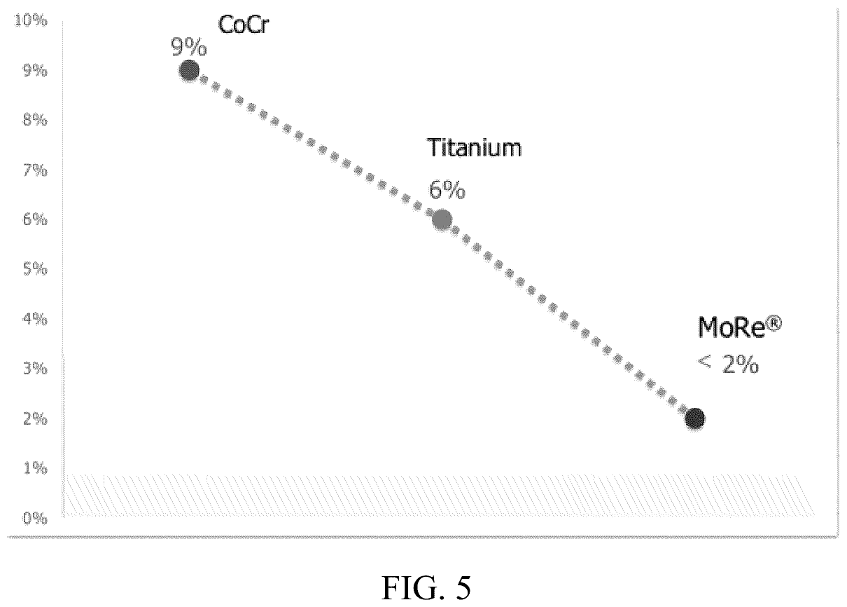

As illustrated in FIGS. 3 and 5, the crimping of a TAV frame that is formed of CoCr alloy (e.g., 35Co-35Ni-20Cr-10Mo) will recoil by 9% or more (e.g., 9-15% and all values and ranges therebetween) after the radial crimping forces are removed from the frame.

As illustrated in FIG. 5, TAV frames formed of refractory metals also have less recoil when crimped and expanded as compared to frames formed of TiAlV alloy. TAV frames formed of Ti alloy (e.g., e.g., Ti-6Al-4V) will recoil by 6% or more (e.g., 6-10% and all values and ranges therebetween) after the radial crimping forces are removed from the frame.

TAV frames formed of refractory metals also have less recoil when crimped expanded as compared to frames formed of stainless steel. TAV frames formed of stainless steel (e.g., 316, 316L) will recoil by 7% or more (e.g., 6-15% and all values and ranges therebetween) after the radial crimping forces are removed from the frame.

Due to the recoil of TAV frames formed of CoCr alloy, stainless steel or TiAlV alloy, the number of crimping cycles required to crimp a TAV frame formed of refractory metal alloy is significantly less than the number of crimping cycles needed to crimp a TAV frame formed of stainless steel, CoCr or TiAlV. Typically, a TAV frame formed of refractory metal alloy requires only one crimping cycles to obtain the desired crimped profile of the TAV frame. Typically, a TAV frame formed of stainless steel, CoCr or TiAlV requires at least two and generally three of more crimping cycles to obtain the desired crimped profile of the TAV frame. Due to such recoil of frames formed of stainless steel, CoCr alloy or TiAlV alloy, the frame of the TAV must be repeatedly subjected to a crimping force to attempt to obtain the smallest crimping outer diameter of the crimped frame. The need to subject the TAV frame to multiple crimping cycles or procedures can potentially result in damage to the frame, damage the leaflets of the TAV, damage the inner and/or outer skirt on the TAV, and/or damage to other components of the medical device (e.g., damage to balloon on the catheter, damage to one or more components on the catheter, etc.).

For example, due to the recoil of CoCr alloy, the TAV frame made of CoCr alloy after one crimping cycle is no more than 91% of the smallest crimping profile, thus requiring at least three crimping cycles to obtain a crimping profile that is 98% or more of the smallest crimping profile of the TAV frame. Also, due to the recoil of TiAlV alloy, the TAV frame made of TiAlV alloy after one crimping cycle is no more than 94% of the smallest crimping profile, thus requiring at least two crimping cycles to obtain a crimping profile that is 98% or more of the smallest crimping profile of the TAV frame. Due to the very small recoil of a refractory metal alloy such as Mo—Re alloy, the TAV frame made of a refractory metal alloy only requires a single crimping cycle to obtain 98-99.9% of the smallest crimping profile of the TAV frame.

A frame formed of a refractory metal alloy such as MoRe alloy was found to have less recoil after being expanded than a frame formed of CoCr alloy. Specifically, it was found that a frame formed of MoRe alloy had a recoil of less than 2% after expansion has compared to a frame formed of CoCr alloy that had 9% or more recoil after expansion. Generally, frames formed of a refractory metal alloy have a recoil that is 1.5-8 times less (and all values and ranges therebetween) than a frame formed of a CoCr alloy, stainless steel, or a TiAlV alloy. As such, a frame formed of a refractory metal alloy will better conform to the shape of the heart passageway wherein the frame is expanded, thus reducing the amount of paravalvular or paraprosthetic leak (PVL) about the prosthetic heart valve after expansion. Furthermore, a frame formed of a refractory metal alloy will expand to its desired expanded state from a single inflation of the balloon of the balloon delivery catheter. Due to the significant recoil of a frame formed of CoCr alloy, stainless steel, and TiAlV alloy after expansion, the balloon of the balloon delivery catheter typically needs to be inflated multiple times to cause the frame to conform to the shape of the heart passageway wherein the frame is expanded. Such multiple inflations of the balloon can result in increased incidence of damage to the prosthetic heart valve and/or to the heart passageway wherein the frame is expanded.

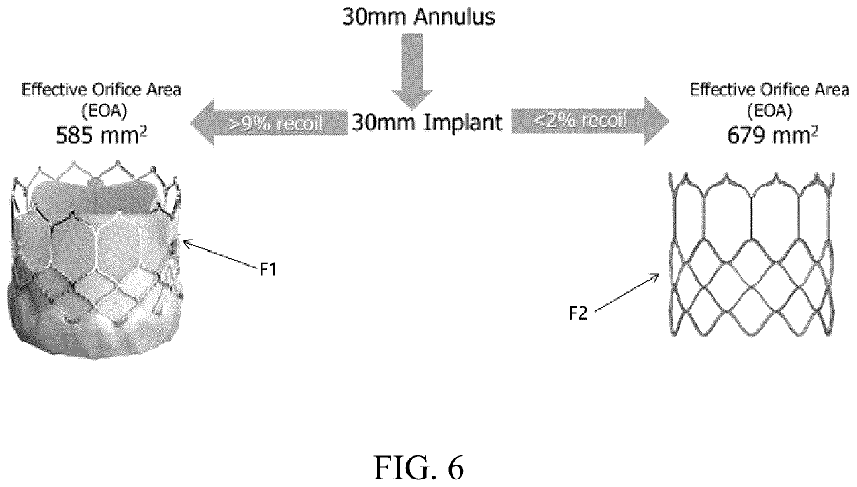

FIG. 6 illustrates two frames for a TAV that are configured to be expanded in a 30 mm annulus of a heart. The maximum cross-sectional area of a 30 mm is 706.86 mm². TAV frame F1 on the left is formed of a CoCr alloy (e.g., MP35N, etc.) and TAV frame F2 on the right is formed of a refractory metal alloy (e.g., 40-60 wt. % Mo, 40-60 wt. % Re, 0-10 wt. % one or more metal additives). When TAV frame F1 formed of CoCr alloy is expanded to a 30 mm diameter by an inflatable balloon, the TAV frame recoils by more than 9% to a diameter of less than 27.3 mm. As such, the maximum cross-sectional area of the expanded TAV frame F1 or the effective orifice area (EOA) after being initially expanded is less than 585.35 mm². When TAV frame F2 formed of refractory metal alloy is expanded to a 30 mm diameter by an inflatable balloon, the TAV frame recoils by less than 2% to a diameter of greater than 29.4 mm. As such, the maximum cross-sectional area of the expanded TAV frame F2 or the effective orifice area (EOA) after being initially expanded is greater than 678.87 mm². The reduction in recoil after the expansion of the TAV frame formed of refractory metal alloy results in the TAV frame of the better conforming to the size of the orifice in the body passageway or organ (e.g., heart, etc.). As such, the increased EOA results in a reduction of perivalvular leak (i.e., a leak caused by a space between the patient's natural heart tissue and the valve replacement). The larger recoil of the TAV frame formed of CoCr alloy results in reduced EOA and increase amount of perivalvular leak about the prosthetic heart valve. Similar issues exist for the large recoil values of stainless frames (7+% recoil) and TiAlV alloy frames (6+% recoil).

As illustrated in FIGS. 3-6, the amount of recoil of a TAV frame formed of refractory metal alloy after being crimped is significantly less than the amount of recoil of a TAV frame formed of CoCr alloy, and TiAlV alloy after being crimped. Also, the amount of recoil of a TAV frame formed of refractory metal alloy after being expanded is significantly less than the amount of recoil of a frame formed of stainless steel, CoCr, and TiAlV alloy after being expanded. Such reduction in the amount of recoil of the TAV frame form of refractory metals as compared to TAV frames formed from stainless steel, CoCr and TiAlV alloys represents a significant advancement in the manufacture and properties of TAVs.

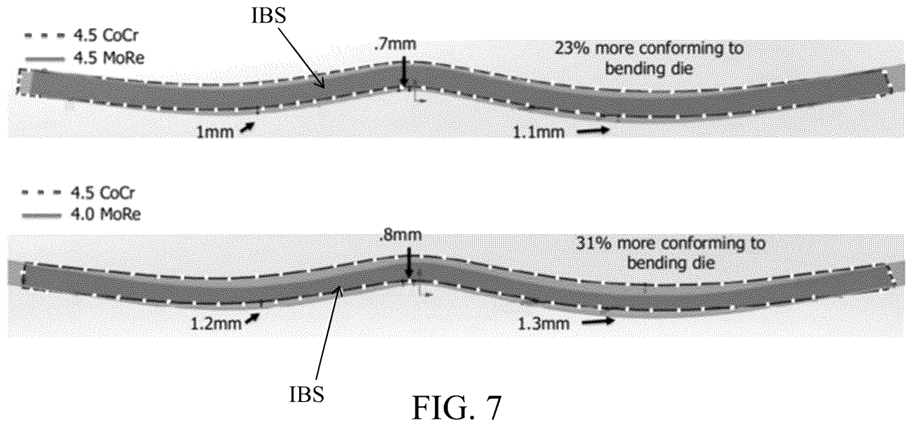

FIG. 7 illustrates two different wires formed of CoCr and a refractory metal such as MoRe to illustrate the conformability to bending of the two types of wires. When the TAV frame is expanded, the struts and posts of the TAV frame plastically deform (e.g., generally deform outwardly) due to the expansion of the inflatable balloon or from some other expansion device. Generally, the treatment location where the TAV device is expanded is not perfectly cylindrical nor has a perfectly shaped circular cross-sectional shape. Generally, the treatment area is damaged and/or includes plaque, calcium deposits and/or other materials (e.g., prior implanted medical devices, etc.) that cause the shape of the treatment area to be non-cylindrical-shaped or have a non-circular cross-sectional shape. As such, TAV frames that can better conform to the irregular shapes in a treatment location result in a TAV that better fits the treatment area and can result in a reduction of perivalvular leak or other types of leakage about the outer perimeter of the expanded TAV. It has been found that refractory metal alloys (e.g., MoRe alloys, etc.) better conform to bending that metal alloys such as stainless steel, CoCr, Nitinol, and TiAlV alloys.

FIG. 7 illustrates that when a MoRe rod and a CoCr rod are subjected to the same bending force, the MoRe wire better conforms to the ideal bending shape IBS than the CoCr wire. The bent rods represented by the top portion of FIG. 7 illustrate that when the two wires were bent, the CoCr wire had a greater deviation from the ideal bent shape region than the wire formed of refractory metal alloy such a MoRe. The two wire bending tests illustrate that the rod formed of refractory metal alloy such a MoRe had 23% and 31% better conformity to the ideal bending shape than the wire formed of CoCr. The ability to conform to a specific shape is largely dependent upon the recoil associated with the material.

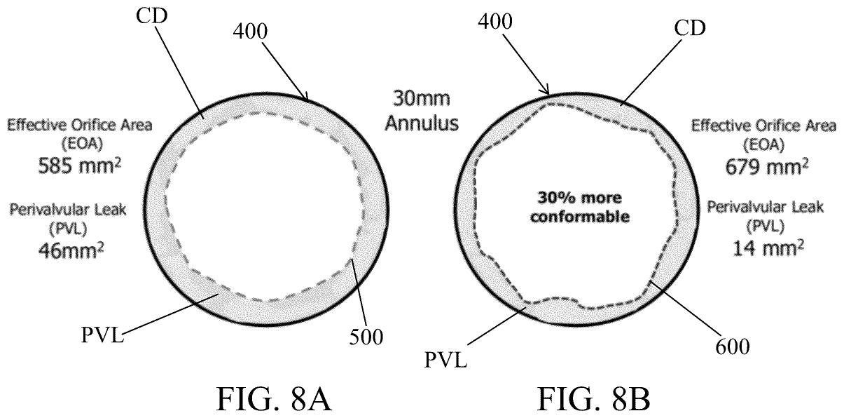

It has been found that wires formed of the refractory metal alloy (e.g., MoRe alloy, ReW alloy, MoReCr alloy, MoReTa alloy, MoReTi alloy, WCu alloy, ReCr alloy, Mo alloy, Re alloy, W alloy, Ta alloy, Nb alloy) have about 15-45% (and all values and ranges therebetween) better conformity to bending to an idea bending shape formed by a die than the same sized wire formed of stainless steel, CoCr alloy, and TiAlV alloy. Such better shape conformity exhibited by the refractory metal alloy is believed to be due in part to the reduced recoil of the refractory metal alloy and one or more other properties of the refractory metal alloy (e.g., strength of alloy, etc.). Such improved shape conformity of refractory metal alloys results in improved conformity of an expanded TAV frame formed of refractory metal alloy to the treatment area shape compared to frames formed of traditional metal alloys as illustrated in FIGS. 8A and 8B.

FIG. 8A illustrates the conformability of an expanded TAV frame 500 formed of CoCr alloy in an irregularly shaped annulus 400 of a heart wherein the treatment area includes calcium deposits CD and leakage regions PVL about the outer perimeter of the expanded TAV frame. The expanded TAV frame forms an EOA of about 585 mm². Due to the inability of the CoCr alloy to readily conform to irregular shapes in the annulus, an open area of about 46 mm² is located about the outer perimeter of the expanded frame of the TAV to allow for PVL about the expanded TAV.

FIG. 8B illustrates the conformability of an expanded TAV frame 600 formed of a refractory metal alloy such as MoRe in an irregularly shaped annulus 400 of a heart. The expanded TAV frame forms an EOA of about 679 mm² due to the improved ability of the refractory metal alloy to conform to irregular shapes in the annulus. As such, only an area of about 14 mm² is located about the outer perimeter of the expanded frame of the TAV. The expanded TAV frame formed of refractory metal alloy is illustrated as being more than 30% (e.g., 30-40% and all values and ranges therebetween) more conformable to the irregularly shaped annulus 400 of a heart as compared to an expanded frame of the TAV formed of CoCr alloy. Such improved conformity also results in nearly 70% less open area about the expanded frame of the TAV that is formed of a refractory metal alloy as compared to an expanded frame of a TAV formed of CoCr alloy. In general, expandable TAV frames form of refractory metal alloy are about 10-50% (and all values and ranges therebetween) more conformable to irregularly shaped body passageways as compared to an expanded TAV frame formed of stainless steel, CoCr alloy, or NiTi alloys.

Because the expandable TAV frame formed of a refractory metal alloy such as MoRe alloy betters conform to the shape of a body passageway wherein the TAV frame is expanded, there is a reduction in the amount of paravalvular or paraprosthetic leak (PVL) or other type of leakage about the TAV after expansion. Furthermore, an expandable TAV frame formed of a refractory metal alloy such as MoRe alloy will expand to its desired expanded state from a single inflation of the balloon of a balloon delivery catheter. Due to the significant recoil of an expandable TAV frame formed of stainless steel, CoCr alloy, or TiAlV alloy after expansion, the balloon of the balloon delivery catheter typically needs to be inflated multiple times to cause the expandable frame to conform as close as possible to the shape of the body passageway wherein the expandable frame is expanded. Such multiple inflations of the balloon can result in increased incidence of damage to the medical device and/or to the body passageway wherein the expandable frame is expanded.

The reduced amount of recoil, improved bending conformity and greater radial strength of expanded TAV frames that are at least partially formed of refractory metal alloy as compared to expandable TAV frames formed of stainless steel, CoCr alloy, and TiAlV alloy results in the following non-limiting advantages: 1) formation of a frame for a medical device having thinner posts, struts, and/or strut joints which results in i) safer vascular access when inserting the medical device through a body passageway and to the treatment area, and/or ii) decreased the risk of bleeding and/or damage to the body passageway and/or the treatment area when the medical device is delivered to the treatment area and/or expanded at the treatment area; 2) easier deliverability of the medical device to the treatment area which can result in i) decreased trauma to the body passageway (e.g., blood vessel, aortic arch trauma, etc.) during the insertion and/or expansion of the medical device at the treatment area, and/or ii) decreased risk of neuro complications-stroke; 3) less recoil which results in i) reduced crimping profile size, ii) increased conformability of the expanded medical device at the treatment area after expansion in the treatment area, iii) increased radial strength of the frame of the medical device after expansion at the treatment area, iv) only require a single crimping cycle to crimp the medical device on a balloon catheter or other type of delivery device, v) reduced incidence of damage to components of the medical device (e.g., struts, posts, strut joints, and/or other components of the expandable frame, leaflets, skirts, coatings, etc.) during the crimping, expansion, and operation of the medical device, vi) greater effective orifice area (EOA) of the medical device after expansion of the medical device, vi) decreased pulmonary valve regurgitation (PVR) after expansion of the medical device in the treatment area, and/or vii) require only a single expansion cycle of the balloon on the balloon catheter or other expansion mechanism to fully expand the medical device; and/or 4) creating a medical device having superior material biologic properties to I) improved tissue adhesion and/or growth on or about medical device, II) reduced adverse tissue reactions with the medical device, III) reduced toxicity of medical device, IV) potentially decreased in-valve thrombosis during the life of the medical device, and/or V) reduced incidence of infection during the life of the medical device.

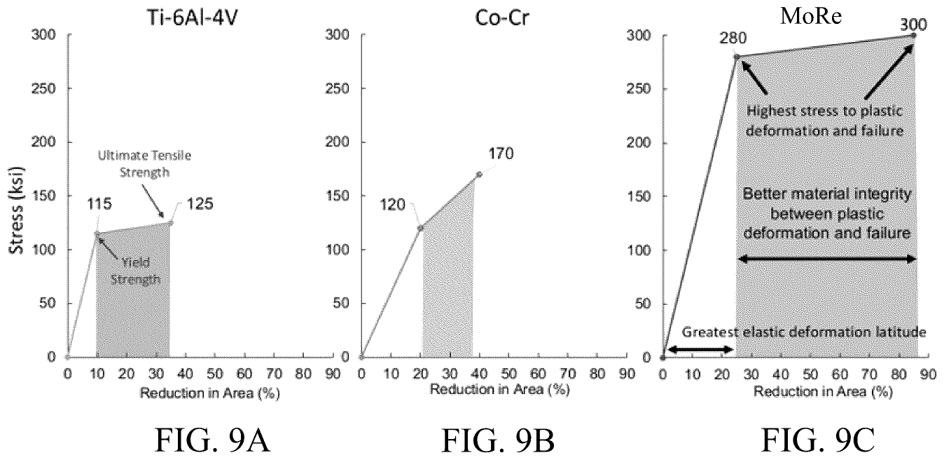

FIGS. 9A-9C illustrate stress vs. reduction in percent area graphs of a TiAlV alloy, a CoCr alloy, and a MoRe alloy. These graphs illustrate that a TAV frame formed of a refractory metal alloy such as MoRe as illustrated in FIG. 9C has improved properties such as strength, yield strength, ultimate tensile strength, fatigue ductility, greater deformation latitude, material integrity between plastic deformation and failure, and durability as compared to materials such a CoCr alloys (FIG. 9B) and TiAlV alloys (FIG. 9A). A refractory metal alloy such as MoRe can have a strength of 1.5-5 times (and all values and ranges therebetween) greater than that of CoCr alloys and TiAlV alloys. Although not illustrated, the refractory metal alloys such as MoRe alloy have improved properties such as strength, yield strength, ultimate tensile strength, fatigue ductility, greater deformation latitude, material integrity between plastic deformation and failure, and durability as compared to stainless steel.

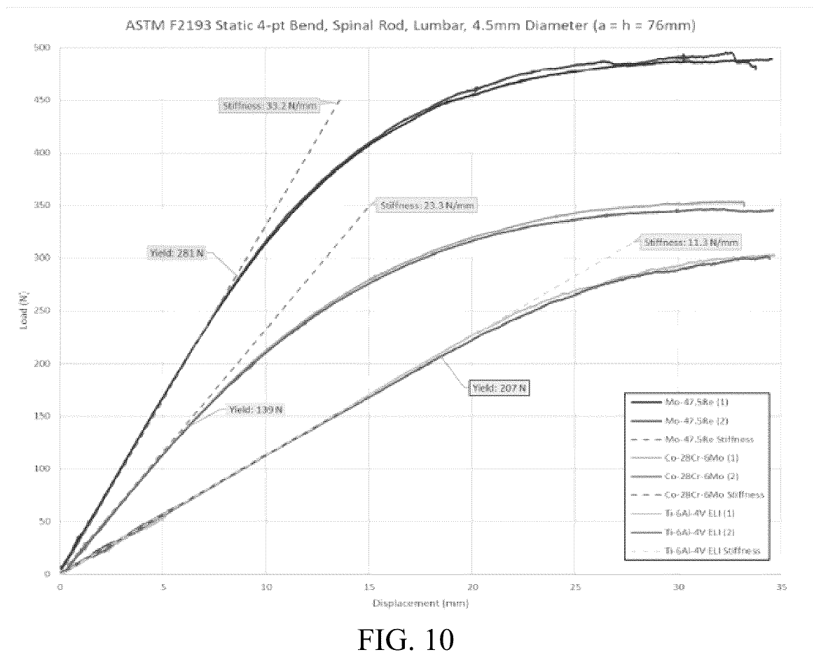

As illustrated in FIG. 10, refractory metal alloys such as MoRe alloy have a greater stiffness and yield strength compared to CoCr alloys and TiAlV alloys. The top curve of FIG. 10 is a MoRe alloy that includes 47.5 wt. % Re and the balance Mo. The middle curve of FIG. 10 is a CoCr alloy that includes 28 wt. % Cr, 6 wt. % Mo and the balance Co. The bottom curve of FIG. 10 is a TiAlV alloy that includes 6 wt. % Al, 4 wt. % V and the balance Ti. Although not illustrated, refractory metal alloys such as MoRe alloy have a greater stiffness and yield strength compared to stainless steel.

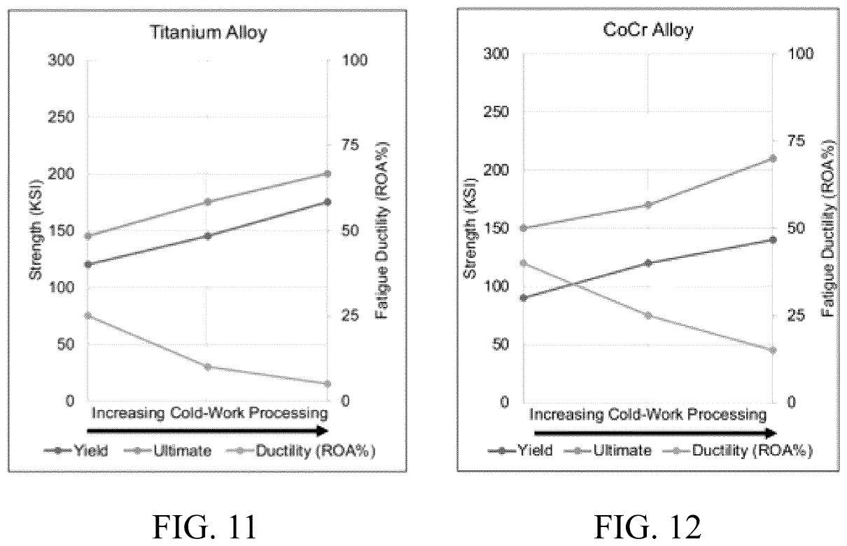

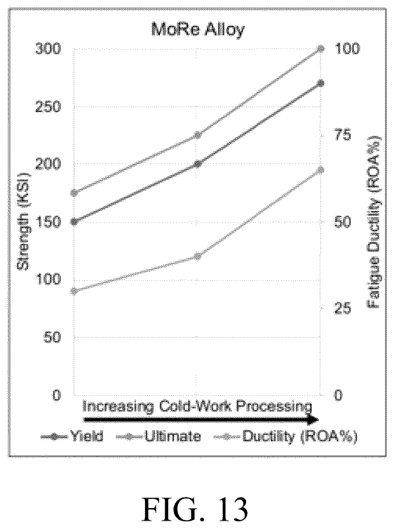

FIGS. 11-13 are graphs that illustrate the yield strength, ultimate strength, and fatigue ductility of a TiAlV alloy, CoCr alloy, and MoRe alloy after such alloys are cold worked to reduce the cross-sectional area of the alloy. After being cold worked, a refractory metal alloy such as MoRe alloy has greater fatigue ductility, yield strength, and ultimate strength than CoCr alloys and TiAlV alloys. Also, the cold working of the MoRe alloy results in the increased ductility of the alloy, wherein CoCr alloys and TiAlV alloys have a reduction in ductility as additional cold working is applied to the alloy. Although not illustrated, the same is true for a refractory metal alloy as compared to stainless steel.

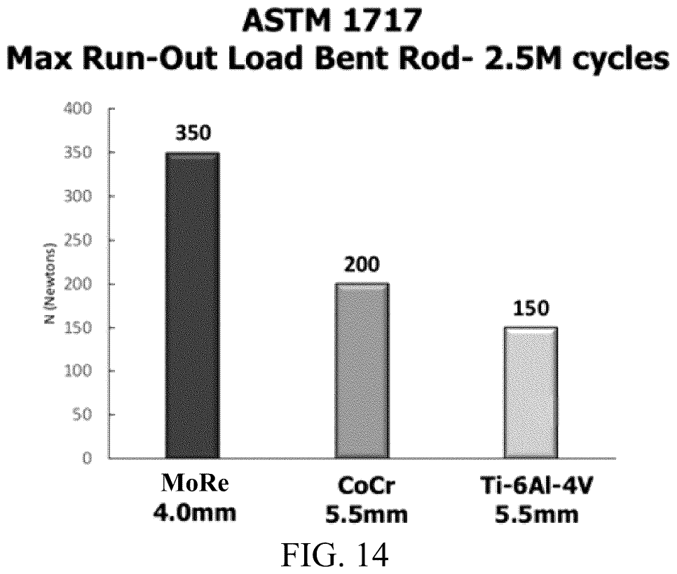

FIG. 14 illustrates that a refractory metal alloy such as MoRe alloy has a greater durability than CoCr alloys and TiAlV alloys. As such, the longevity of a TAV frame formed of a refractory metal that is subjected to bending stresses when inserted into a patient is greater than the longevity of a TAV frame formed of CoCr and TiAlV alloys. Although not illustrated, the same is true for a refractory metal alloy as compared to stainless steel.

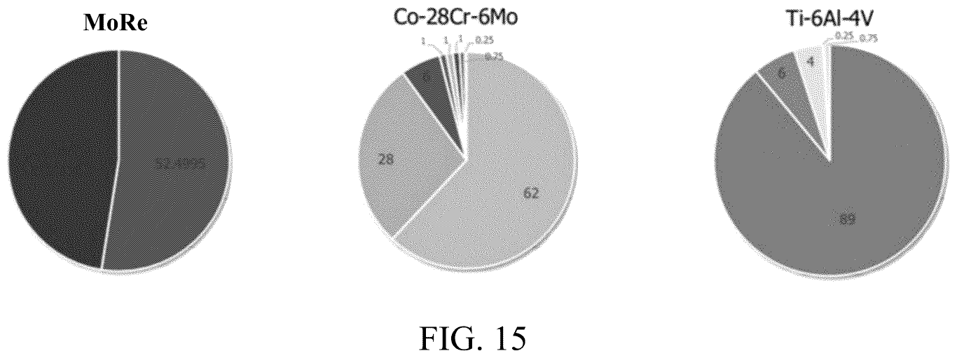

FIG. 15 compares the purity level of a refractory metal alloy such as MoRe alloy to alloys such as CoCr alloys and TiAlV alloys. The purity level of a refractory metal such as MoRe is significantly greater than CoCr alloys and TiAlV alloys. The MoRe alloy is illustrated as including 47.4995 wt. % molybdenum and 52.4995 wt. % rhenium. The CoCr alloy includes 62 wt. % cobalt, 28 wt. % chromium, 6 wt. % molybdenum, and impurities such as 1 wt. % nickel, 1 wt. % silicon, 1 wt. % manganese, 0.25 wt. % iron, and 0.75 wt. % other elements. The TiAlV alloy includes 89 wt. % titanium, 6 wt. % aluminum, 4 wt. % vanadium, and impurities such as 0.25 wt. % iron, and 0.75 wt. % other elements. It is believed that impurities in an alloy can result in reduced fatigue life of the alloy, adverse tissue reactions when exposed to the alloy, undesirable metal ion release from the alloy, and/or allergic reactions when tissue and/or fluids are exposed to the alloy. The use of refractory metals for the metal device overcomes these potential issues that other alloys have with regard to impurity content of the alloy.

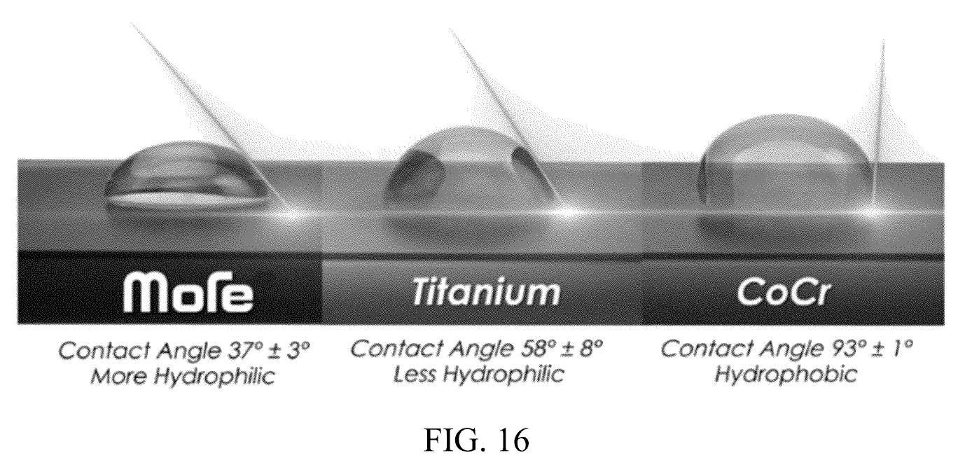

FIG. 16 illustrates the hydrophilicity of a refractory metal alloy such as a MoRe alloy compared to a CoCr alloy or TiAlV alloy. Hydrophilicity of a material implanted in a patient is an important property of the material with regard to the cell adhesion, cell migration, and cell multiplication of tissue on the material. As illustrated in FIG. 16, CoCr alloys are hydrophobic materials resulting in a large contact angle (93°±1) of a water droplet (e.g., distilled water) positioned on the surface of the CoCr alloy. TiAlV alloys are a little more hydrophilic than CoCr alloys and exhibit a contact angle of 58°±8° when a water droplet is positioned on the surface of the Ti alloy. Refractory metal alloys such as a MoRe alloy have a much greater hydrophilicity than CoCr alloys and TiAlV alloys. The MoRe alloy has a contact angle of 37°±3 when a water droplet is positioned on the surface of the MoRe alloy. The refractory metal alloys generally have a hydrophilicity wherein the contact angle of a water droplet on the surface of the refractory metal alloy is 25°-45° (and all values and ranges therebetween), and typically 30-42°.

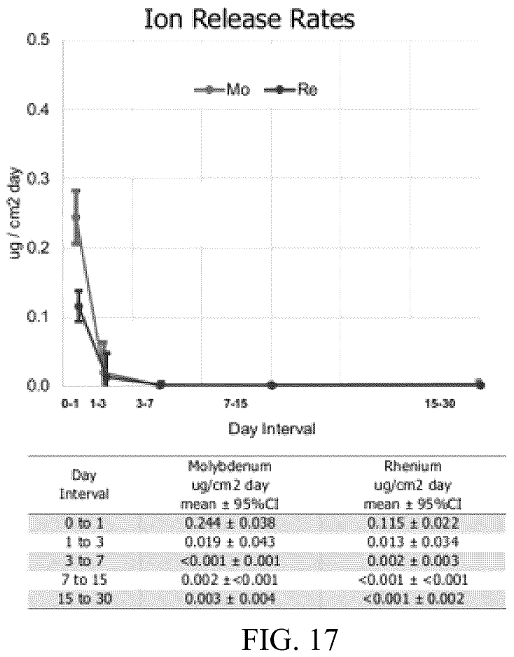

FIGS. 17-19 illustrate the ion release of a refractory metal alloy such as MoRe alloy. As illustrated in FIG. 17, during the first day of implanting the medical device in a patient, the ion release of molybdenum is about 0.244 μg/cm² per day and the ion release of rhenium is about 0.115 μg/cm² per day. From days 1-3, the ion release of molybdenum is about 0.019 μg/cm² per day and the ion release of rhenium is about 0.013 μg/cm² per day. From days 3-7, the ion release of molybdenum is less than 0.001 μg/cm² per day and the ion release of rhenium is about 0.002 μg/cm² per day. From days 7-15, the ion release of molybdenum is about 0.002 μg/cm² per day and the ion release of rhenium is less than 0.001 μg/cm² per day. From days 15-30, the ion release of Mo is about 0.003 μg/cm² per day and the ion release of rhenium is less than 0.001 μg/cm² per day. The graph illustrates that after the seventh day of implantation in tissue, the ion release of molybdenum and rhenium from the MoRe alloy is effectively nonexistent.

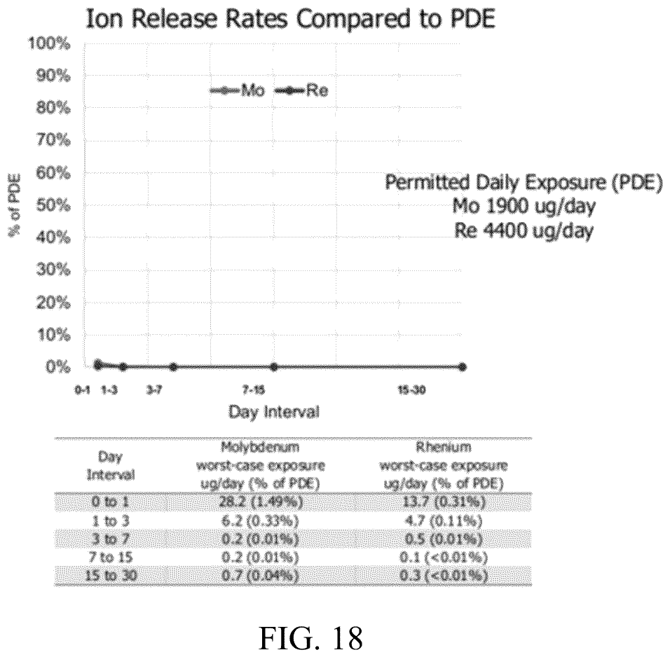

FIG. 18 illustrates that the ion release of molybdenum from the MoRe alloy is less than 1.5% of the allowed daily exposure to molybdenum during the first day of insertion of the MoRe alloy in a patient, and such daily ion exposure of molybdenum drops to 0.04% of the allowed daily exposure after 15 days. FIG. 18 also illustrates that the ion release of rhenium from the MoRe alloy is less than 0.31% of the allowed daily exposure to rhenium during the first day of insertion of the MoRe alloy in a patient, and such daily ion exposure of rhenium drops to less than 0.01% of the allowed daily exposure after 15 days.

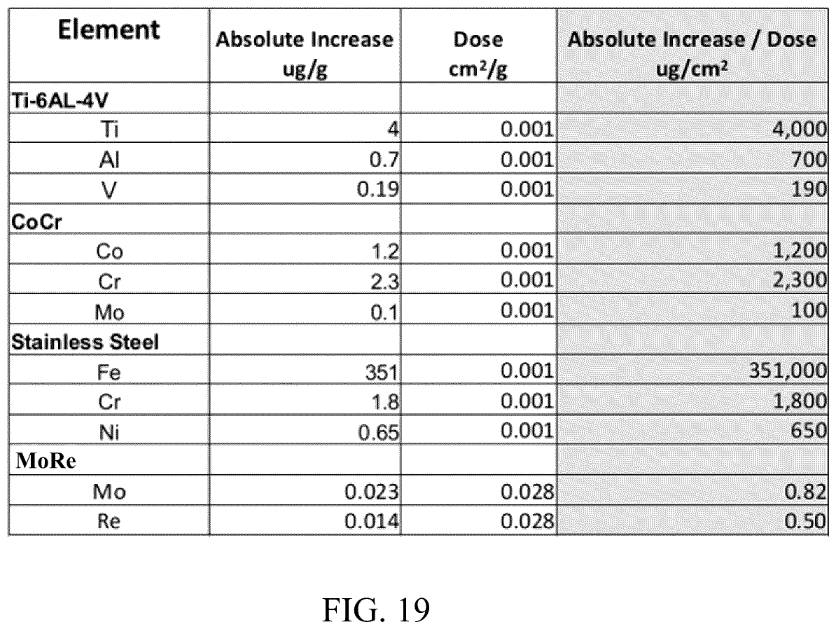

FIG. 19 is a table that illustrates the amount of the primary metals in the alloys of TiAlV, CoCr, stainless steel, and MoRe released into a patient after the medical device including such alloys is inserted into a patient for 90 days. As illustrated in the table, the amount of molybdenum and rhenium contained in the tissue surrounding the MoRe alloy after 90 days is significantly lower than any of the primary metals of the other alloys. The amount of molybdenum metal ion in a gram of tissue from a 0.028 cm²/g dose of MoRe in the tissue after 90 days is 0.023 μg/g. As such, the absolute increase in molybdenum metal ion relative to the dose size of the MoRe alloy in the tissue was 0.82. The amount of rhenium metal ion in a gram of tissue from a 0.028 cm²/g dose of MoRe in the tissue after 90 days is 0.014 μg/g. As such, the absolute increase in Re metal ion relative to the dose size of the MoRe alloy in the tissue was 0.5. Based on absolute increase in metal ions in the tissue relative to the dose of the metal alloy in the tissue, both the molybdenum and rhenium content in the tissue form the MoRe alloy after the MoRe alloy was implanted in the tissue for 90 days was over 120 times less than the molybdenum from the CoCr alloy, and many more times less than the other primary metals of the tested alloys. Generally, the absolute ion release of the primary elements of the refractory metal alloys (e.g., primary is an element in an alloy that is at least 2 wt. % of the alloy) relative to the dose of the refractory metal alloy in the tissue after 90 days is at least 120 times less than any of the primary components of the alloys of TiAlV, CoCr, stainless steel. As such, less potentially irritating metal ions are released from the refractory metal alloy compared to CoCr alloys, TiAlV alloys, and stainless steel. In general, the refractory metal alloy has a maximum ion release of a primary component of said refractory metal alloy when inserted or implanted on or in the body of the patient of no more than 0.5 μg/cm² per day (e.g., 0-0.5 μg/cm² per day and all values and ranges therebetween). The primary component of the refractory metal alloy is a metal that constitutes at least 2 wt. % of the refractory metal alloy. Also, the refractory metal alloy, when implanted into tissue for at least 90 days, has an absolute increase in ion release per dose of refractory metal alloy in the tissue about said medical device no more than 50 (e.g., 0.01-50 and all values and ranges therebetween).

Medical devices, such as expandable heart valves that are at least partially formed of the refractory metal in accordance with the present disclosure, overcome several unmet needs that exist in expandable medical device formed of CoCr alloys, TiAlV alloys, and stainless steel. Such unmet needs addressed by the medical devices in accordance with the present disclosure include 1) not having to form a large hole in large arterial vessels or other blood vessels for initial insertion of the crimped medical device into the atrial vessel or other blood vessel, thereby reducing the incidence of lethal bleeding during a treatment; 2) enabling the medical device to be delivered and implanted in abnormally shaped heart valves or through an abnormally shaped arterial vessel due to calcination in the prosthetic heart valve and/or calcination and/or plaque in the arterial vessel by creating a medical device (e.g., stent, prosthetic heart valve, etc.) having a reduced crimped profile that is smaller than medical devices formed of CoCr alloys, TiAlV alloys, and stainless steel; 3) reducing the incidence of a perivalvular leak and/or other types of leakage about the implanted medical device when the medical device is expanded in the treatment region by using a frame formed of the refractory metal alloy that better conforms to the shape of the abnormally shaped heart valve orifice upon expansion of the prosthetic heart valve comparted to prior art prosthetic heart valves formed of CoCr alloys, TiAlV alloys, and stainless steel, thereby reducing the incidence of stroke and/or by increasing the incidence of success of the implanted medical device; 4) improving the radial strength of the expanded struts, posts, and/or strut joints in the expandable frame and the strength of the expandable frame itself after expansion the medical device; 5) reducing the amount of recoil of the expandable frame during the crimping and/or expansion of the expandable frame of the medical device; 6) enabling the medical device to be used in a heart that has a permanent pacemaker; 7) reducing the incidence of minor stroke during the insertion and operation of the medical device at the treatment area; 8) reducing the incidence of coronary ostium compromise; 9) improving foreshortening; 10) reducing further aortic valve calcification and/or calcification in a blood vessel after implantation of the medical device; 11) reducing the need for multiple crimping cycles when inserting the medical device on a catheter or other type of delivery system; 12) reducing the incidence of frame/stent fracture during the crimping and/or expansion of the medical device; 13) reducing the incidence of biofilm-endocarditis after implantation of the medical device; 14) reducing allergic reactions to the medical device after implantation of the medical device; 15) improving the hydrophilicity of the medical device to improve tissue growth on and/or about the implanted medical device, 16) reduce the magnetic susceptibility of the medical device, 17) reduce the toxicity of the medical device, 18) reduce the amount of metal ion release from the medical device, and/or 19) increasing the longevity of leaflets and/or stent/frame and/or other components of the medical device after insertion of the medical device.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall therebetween.

To aid the Patent Office and any readers of this application and any resulting patent in interpreting the claims appended hereto, applicants do not intend any of the appended claims or claim elements to invoke 35 U.S.C. 112(f) unless the words “means for” or “step for” are explicitly used in the particular claim.

1-39. (canceled)

40. A medical device for implantation into a body passageway; said medical device includes an expandable metal frame that is configured to expand in the body passageway when said medical device is positioned in a treatment site in the body passageway; at least 50 wt. % of said expandable metal frame is formed of a refractory metal alloy; said refractory metal alloy is not a self-expanding metal alloy; said expandable metal frame of said medical device includes a plurality of struts and strut joints; said expandable metal frame includes two or more features selected from the group consisting of a) a recoil percentage of less than 5% when said expandable metal frame is crimped to a crimped state, b) a recoil percentage of less than 5% when said expandable metal frame is expanded from a crimped state, and c) a longitudinal foreshortening percentage of less than 20% when said expandable metal frame is expanded from a crimped state; said refractory metal alloy has a density of at least 11 gm/cc; said refractory metal alloy includes at least 50 wt. % of a total content of refractory metal and 0-40 of additive metal; said refractory metal includes alloy includes one or more of molybdenum, niobium, rhenium, tantalum, and/or tungsten; said refractory metal alloy includes no less than 30 wt. % rhenium and one or both of niobium and tantalum; said additive metal includes one or more of calcium, carbon, chromium, cobalt, copper, gold, hafnium, iridium, iron, lanthanum, magnesium, manganese, nickel, osmium, platinum, rare earth metals, rhodium, ruthenium, silver, technetium, titanium, vanadium, yttrium, zinc, and/or zirconium; said refractory metal alloy includes no more than 2 wt. % of a combination of a) metals other than sad refractory metal and additive metal, b) carbon, c) oxygen and d) nitrogen.

41. The medical device as defined in claim 40, wherein said refractory metal alloy includes 1-32 wt. % niobium and/or 0.1-32 wt. % tantalum.

42. The medical device as defined in claim 40, wherein said refractory metal alloy includes 30-60 wt. % rhenium.

43. The medical device as defined in claim 41, wherein said refractory metal alloy includes 30-60 wt. % rhenium.

44. The medical device as defined in claim 41, wherein said refractory metal alloy includes 1-55 wt. % molybdenum.

45. The medical device as defined in claim 43, wherein said refractory metal alloy includes 1-55 wt. % molybdenum.

46. The medical device as defined in claim 41, wherein said refractory metal alloy includes 1-32 wt. % chromium.

47. The medical device as defined in claim 43, wherein said refractory metal alloy includes 1-32 wt. % chromium.

48. The medical device as defined in claim 45, wherein said refractory metal alloy includes 1-32 wt. % chromium.

49. The medical device as defined in claim 40, wherein said expandable metal frame has i) a recoil percentage of no more than 2% when said expandable metal frame is crimped to said crimped state, ii) a recoil percentage of no more than 2% when said expandable metal frame is expanded from said crimped state, and iii) a longitudinal foreshortening percentage of no more than 15% when said expandable metal frame is expanded from said crimped state.

50. The medical device as defined in claim 40, wherein said refractory metal alloy I) has a hydrophilicity wherein a contact angle of a water droplet on a surface of said refractory metal alloy is 25-45°, and/or II) said refractory metal alloy has a maximum ion release of a primary component of said refractory metal alloy when inserted or implanted on or in the body of the patient of no more than 0.5 μg/cm² per day, and wherein said primary component constitutes at least 2 wt. % of said refractory metal alloy.

51. The medical device as defined in claim 40, wherein said medical device is an expandable stent, an expandable valve, an expandable graph, an expandable sheath or an expandable frame for a prosthetic heart valve.

52. The medical device as defined in claim 51, wherein said medical device is a prosthetic heart valve; said prosthetic heart valve includes said expandable metal frame, a leaflet structure supported by said expandable metal frame, and an inner skirt secured to said expandable metal frame.

53. A medical device for implantation into a body passageway; said medical device is an expandable stent, an expandable valve, an expandable graph, an expandable sheath or an expandable frame for a prosthetic heart valve; at least 50 wt. % of said medical device is formed of a refractory metal alloy; said refractory metal alloy is not a self-expanding metal alloy; said medical device includes an expandable metal frame formed of a plurality of struts and strut joints; said expandable metal frame includes two or more features selected from the group consisting of a) a recoil percentage of less than 5% when said expandable metal frame is crimped to a crimped state, b) a recoil percentage of less than 5% when said expandable metal frame is expanded from a crimped state, and c) a longitudinal foreshortening percentage of less than 20% when said expandable metal frame is expanded from a crimped state; said refractory metal alloy has a density of at least 11 gm/cc; said refractory metal alloy includes at least 50 wt. % of a total content of refractory metal and 0-40 of additive metal; said refractory metal includes alloy includes one or more of molybdenum, niobium, rhenium, tantalum, and/or tungsten; said refractory metal alloy includes no less than 30 wt. % rhenium and one or both of niobium and tantalum; wherein said refractory metal alloy includes 1-32 wt. % niobium and/or 0.1-32 wt. % tantalum; said additive metal includes one or more of calcium, carbon, chromium, cobalt, copper, gold, hafnium, iridium, iron, lanthanum, magnesium, manganese, nickel, osmium, platinum, rare earth metals, rhodium, ruthenium, silver, technetium, titanium, vanadium, yttrium, zinc, and/or zirconium; said refractory metal alloy includes no more than 2 wt. % of a combination of a) metals other than sad refractory metal and additive metal, b) carbon, c) oxygen and d) nitrogen.

54. The medical device as defined in claim 53, wherein said refractory metal alloy includes 30-60 wt. % rhenium.

55. The medical device as defined in claim 53, wherein said refractory metal alloy includes 1-55 wt. % molybdenum.

56. The medical device as defined in claim 54, wherein said refractory metal alloy includes 1-55 wt. % molybdenum.

57. The medical device as defined in claim 53, wherein said refractory metal alloy includes 1-32 wt. % chromium.

58. The medical device as defined in claim 54, wherein said refractory metal alloy includes 1-32 wt. % chromium.

59. The medical device as defined in claim 55, wherein said refractory metal alloy includes 1-32 wt. % chromium.

60. The medical device as defined in claim 56, wherein said refractory metal alloy includes 1-32 wt. % chromium.

61. The medical device as defined in claim 53, wherein said expandable metal frame has i) a recoil percentage of no more than 2% when said expandable metal frame is crimped to said crimped state, ii) a recoil percentage of no more than 2% when said expandable metal frame is expanded from said crimped state, and iii) a longitudinal foreshortening percentage of no more than 15% when said expandable metal frame is expanded from said crimped state.

62. The medical device as defined in claim 53, wherein said refractory metal alloy I) has a hydrophilicity wherein a contact angle of a water droplet on a surface of said refractory metal alloy is 25-45°, and/or II) said refractory metal alloy has a maximum ion release of a primary component of said refractory metal alloy when inserted or implanted on or in the body of the patient of no more than 0.5 μg/cm² per day, and wherein said primary component constitutes at least 2 wt. % of said refractory metal alloy.

63. The medical device as defined in claim 53, wherein said medical device is a prosthetic heart valve; said prosthetic heart valve includes said expandable metal frame, a leaflet structure supported by said expandable metal frame, and an inner skirt secured to said expandable metal frame.
